(19) United States
(12) Patent Application Publication
(10) Pub. No.: US 2024/0167029 A1
(43) Pub. Date: May 23, 2024

(54) MODIFIED GUIDE RNAS FOR CRISPR GENOME EDITING

(71) Applicant: UNIVERSITY OF MASSACHUSETTS, Boston, MA (US)

(72) Inventors: Anastasia Khvorova, Westborough, MA (US); Ken Yamada, Worcester, MA (US); Erik Joseph Sontheimer, Auburndale, MA (US)

(21) Appl. No.: 18/213,505

(22) Filed: Jun. 23, 2023

Related U.S. Application Data
(60) Provisional application No. 63/355,704, filed on Jun. 27, 2022.

(51) Int. Cl. C12N 15/113; C12N 9/22; C12N 15/90

(52) U.S. Cl. CPC C12N 15/113; C12N 9/22; C12N 15/907; C12N 2310/20; C12N 2310/315; C12N 2310/321; C12N 2310/3515; C12N 2310/531; C12N 2800/80

(57) ABSTRACT

Extended Nucleic Acid (exNA)-modified crRNAs and tracrRNAs are provided. Methods of using the crRNAs and tracrRNAs for genome editing with a CRISPR nuclease and kits for performing the same are also provided.

### CROSS REFERENCE TO RELATED APPLICATION

This application claims the benefit of U.S. Provisional Patent Application Ser. No. 63/355,704, filed Jun. 27, 2022, the contents of which are incorporated by reference in its entirety for all purpose.

### SEQUENCE LISTING

The instant application contains a Sequence Listing which has been submitted electronically in XML format and is hereby incorporated by reference in its entirety. Said XML file, created on Nov. 10, 2023, is named 741117_UM9-256_ST26.xml and is 780,684 bytes in size.

### FIELD OF THE INVENTION

This disclosure relates to compositions and methods of modified guide RNAs for CRISPR genome editing.

### BACKGROUND

CRISPR RNA-guided genome engineering has revolutionized research into human genetic disease and many other aspects of biology. Numerous CRISPR-based in vivo or ex vivo genome editing therapies are nearing clinical trials. At the heart of this revolution are the microbial effector proteins found in class II CRISPR-Cas systems such as Cas9 (type II) and Cas12a/Cpf1 (type V) (Jinek et al. Science 337, 816-821 (2012); Gasiunas et al. PNAS 109, E2579-E2586 (2012): Zetsche et al. Cell 163, 759-771 (2015)).

The versatility of Cas9 for genome editing derives from its RNA-guided nature. The crRNA of SpCas9 usually includes a 20-nucleotide guide region followed by a 16-nucleotide repeat region (FIG. 1). The tracrRNA consists of an anti-repeat region that pairs with the crRNA, and also includes three stem-loops. All of these secondary structure elements are required for efficient editing in mammalian systems (Hsu et al. Nature Biotechnology 31, 827 (2013). Nevertheless, existing guide RNAs suffer from several limitations, which limit their utility in therapeutic applications. For example, existing guide RNAs may be subject to rapid degradation in circulation and within cells. Moreover, chemical modifications of guide RNAs may reduce stability and editing efficiency. Accordingly, there exists a need in the art for optimized guide RNAs that retain efficient genome editing activity in vivo and ex vivo when paired with a CRISPR nuclease, such as Cas9.

### SUMMARY

The present disclosure provides chemically modified guide RNAs for CRISPR genome editing. In certain embodiments, the guide RNAs of the disclosure are heavily or fully chemically modified. The guide RNA of the disclosure may confer several advantages in vivo or ex vivo, including stability, improved potency, and/or reduced off-target effects. Furthermore, in certain embodiments, the modified RNAs of the disclosure have reduced immunogenicity, e.g., a reduced ability to induce innate immune responses.

In one aspect, the disclosure provides a chemically modified guide RNA comprising: (a) a crRNA portion comprising (i) a guide sequence capable of hybridizing to a target polynucleotide sequence, and (ii) a repeat sequence: and (b) a tracrRNA portion comprising an anti-repeat nucleotide sequence that is complementary to the repeat sequence, wherein the crRNA portion comprises at least one exNA intersubunit linkage.

In certain embodiments, the at least one exNA intersubunit linkage is in the guide sequence.

In certain embodiments, the at least one exNA intersubunit linkage is in the repeat sequence.

In certain embodiments, the crRNA portion comprises the exNA intersubunit linkage between one or more of positions 35 to 36, 32 to 33, 30 to 31, 24 to 25, 23 to 24, 22 to 23, 21 to 22, 17 to 18, 13 to 14, and 8 to 9, from the 5′ end of the crRNA portion.

In certain embodiments, the crRNA portion comprises the exNA intersubunit linkage between one or more of positions 35 to 36, 32 to 33, 30 to 31, 24 to 25, 23 to 24, 22 to 23, 21 to 22, 17 to 18, 13 to 14, and 8 to 9, from the 5′ end of the crRNA portion set forth in NNNNNNNNNNNNNNNNNNNNGUUUUAGAGCUAUGCU (SEQ ID NO: 1), wherein “N” corresponds to any nucleotide (e.g., A, U, G, or C).

In certain embodiments, the exNA intersubunit linkage comprises the intersubunit linkage of Formula Ia:

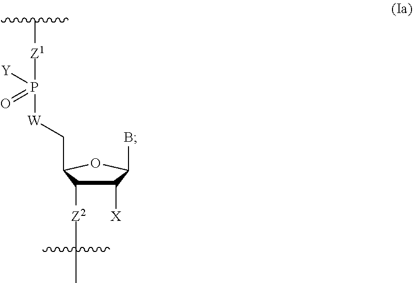

wherein:
B is a base moiety;
W is O or O(CH₂)ₙ¹, wherein n¹ is 1 to 10;
X is selected from the group consisting of H, OH, OR¹, R¹, F, Cl, Br, I, SH, SR¹, NH₂, NHR¹, NR¹₂, and COOR¹;
Y is selected from the group consisting of O⁻, OH, OR², NH⁻, NH₂, NR²₂, BH₃, S⁻, R², and SH;
Z¹ is O or O(CH₂)ₙ² wherein n² is 1 to 10;
Z² is O or O(CH₂)ₙ³ wherein n³ is 1 to 10;
R¹ is a substituted or unsubstituted C₁-C₆ alkyl, alkenyl, alkynyl, aryl, or mixtures thereof; and
R² is a substituted or unsubstituted C₁-C₆ alkyl, alkenyl, alkynyl, aryl, or mixtures thereof, wherein:
Z₁ is O(CH₂)ₙ² and W is O, or Z₁ is O and W is O(CH₂)ₙ¹, or Z₁ is O(CH₂)ₙ² and W is O(CH₂)ₙ¹.

In certain embodiments, Z¹ is O(CH₂)ₙ², n² is 1, W is O, and Y is O⁻. In certain embodiments, Z¹ is O, W is O(CH₂)ₙ¹, n¹ is 1, and Y is O⁻. In certain embodiments, Z¹ is O(CH₂)ₙ², n² is 1, W is O, and Y is O⁻. In certain embodiments, Z¹ is O(CH₂)ₙ², n² is 1, W is O(CH₂)ₙ¹, and Y is O⁻. In certain embodiments, Z¹ is O(CH₂)ₙ², n² is 1, W is O(CH₂)ₙ¹, and Y is S⁻. In certain embodiments, Y is S⁻. In certain embodiments, X is OR¹ or F. In certain embodiments, the base moiety B is selected from the group consisting of adenine, guanine, cytosine, and uracil.

In certain embodiments, the crRNA portion and/or the tracrRNA portion further comprise at least one modified nucleotide selected from a modification of a ribose group, a phosphate group, a nucleobase, or a combination thereof.

In certain embodiments, each modification of the ribose group is independently selected from the group consisting of 2′-O-methyl, 2′-fluoro, 2′-deoxy, 2′-O-(2-methoxyethyl) (MOE), 2′-NH₂ (2′-amino), 4′-thio, a bicyclic nucleotide, a locked nucleic acid (LNA), a 2′-(S)-constrained ethyl (S-cEt), a constrained MOE, and a 2′-O,4′-C-aminomethylene bridged nucleic acid (2′,4′-BNA^{NC}).

In certain embodiments, at least 80% of the ribose groups are chemically modified. In certain embodiments, at least 90% of the ribose groups are chemically modified. In certain embodiments, 100% of the ribose groups are chemically modified.

In certain embodiments, each modification of the phosphate group is independently selected from the group consisting of a phosphorothioate, phosphonoacetate (PACE), thiophosphonoacetate (thioPACE), amide, triazole, phosphonate, and phosphotriester modification.

In certain embodiments, each modification of the nucleobase group is independently selected from the group consisting of 2-thiouridine, 4-thiouridine, N⁶-methyladenosine, pseudouridine, 2,6-diaminopurine, inosine, thymidine, 5-methylcytosine, 5-substituted pyrimidine, isoguanine, isocytosine, and halogenated aromatic groups.

In certain embodiments, the guide RNA comprises at least 90% modified nucleotide. In certain embodiments, the guide RNA comprises 100% modified nucleotides.

In certain embodiments, the chemically modified guide RNA comprises a crRNA portion modification pattern selected from the group consisting of:

| Name (Alt. | |
|---|---|
| Name) | Sequence |
| crRNA Ex20- | mN#mN#mN#mNmNmNmNmNmNmNfNfNfNfNrN#rN#f |
| 1 (36C-ExU) | NfNrN#mNmGrU#rU#rU#fUfAmGmAmGmCmUmAmU# |
| | mG#mC#exmU(SEQ ID NO: 5) |
| crRNA Ex20- | mN#mN#mN#mNmNmNmNmNmNmNfNfNfNfNrN#rN#f |
| 4 (33C-ExU) | NfNrN#mNmGrU#rU#rU#fUfAmGmAmGmCmUmAexmU |
| | #mG#mC#Mu(SEQ ID NO: 6) |
| crRNA Ex20- | mN#mN#mN#mNmNmNmNmNmNmNfNfNfNfNrN#rN#f |
| 5 (31C-ExU) | NfNrN#mNmGrU#rU#rU#fUfAmGmAmGmCexmUmAmU |
| | #mG#mC#Mu(SEQ ID NO: 7) |
| crRNA Ex20- | mN#mN#mN#mNmNmNmNmNmNmNfNfNfNfNrN#rN#f |
| 7 (25C-ExU) | NfNrN#mNmGrU#rU#rU#exfUfAmGmAmGmCmUmAmU |
| | #mG#mC#mU(SEQ ID NO: 8) |
| crRNA Ex20- | mN#mN#mN#mNmNmNmNmNmNmNfNfNfNfNrN#rN#f |
| 8 (24C-ExU) | NfNrN#mNmGrU#rU#exrU#fUfAmGmAmGmCmUmAmU |
| | #mG#mC#mU(SEQ ID NO: 9) |
| crRNA Ex20- | mN#mN#mN#mNmNmNmNmNmNmNfNfNfNfNrN#rN#f |
| 9 (23C-ExU) | NfNrN#mNmGrU#exrU#rU#fUfAmGmAmGmCmUmAmU |
| | #mG#mC#mU(SEQ ID NO: 10) |
| crRNA Ex20- | mN#mN#mN#mNmNmNmNmNmNmNfNfNfNfNrN#rN#f |
| 10 (22C-ExU) | NfNrN#mNmGexrU#rU#rU#fUfAmGmAmGmCmUmAmU |
| | #mG#mC#mU(SEQ ID NO: 11) |
| crRNA Ex20- | mN#mN#mN#mNmNmNmNmNmNmNfNfNfNfNrN#rN#f |
| 12 (18C-ExU) | NexfNrN#mNmGrU#rU#rU#fUfAmGmAmGmCmUmAmU |
| | #mG#mC#mU(SEQ ID NO: 12) |
| crRNA Ex20- | mN#mN#mN#mNmNmNmNmNmNmNfNfNfNexfNrN#rN# |
| 13 (14C-ExU) | fNfNrN#mNmGrU#rU#rU#fUfAmGmAmGmCmUmAmU# |
| | mG#mC#Mu(SEQ ID NO: 13) |
| crRNA Ex20- | mN#mN#mN#mNmNmNmNmNexmNmNfNfNfNfNrN#rN# |
| 15 (9C-ExU) | fNfNrN#mNmGrU#rU#rU#fUfAmGmAmGmCmUmAmU# |
| | mG#mC#mU(SEQ ID NO: 14) |

KEY:
IN = RNA, mN = 2′-O-methyl RNA, fN = 2′-fluoro RNA, NexN = exNA linkage, N = A, U, G, or C N#N = phosphorothioate linkage

In certain embodiments, the chemically modified guide comprises a tracrRNA portion modification pattern selected from any one of tracrRNA 1 to tracrRNA 116 of Table 2 or any one of tracrRNA 2-1 (67T-ExU), tracrRNA 2-2 (66T-ExU), tracrRNA 2-3 (65T-ExU), tracrRNA 2-5 (66/67T-ExU), tracrRNA 2-6 (65/66/67T-ExU), tracrRNA 2-8 (62T-ExU), tracrRNA 2-10 (58T-ExU), tracrRNA 2-14 (48T-ExU), tracrRNA 2-15 (40T-ExU), tracrRNA 2-16 (39T-ExU), tracrRNA 2-19 (34T-ExU), tracrRNA 2-20 (32T-ExU), tracrRNA 2-21 (31T-ExU), tracrRNA 2-23 (27T-ExU), tracrRNA 2-24 (24T-ExU), tracrRNA 2-25 (18T-ExU), tracrRNA 2-26 (13T-ExU), tracrRNA 2-27 (12T-ExU), tracrRNA 2-30 (5T-ExU), tracrRNA 2-31 (12/24/3′T-ExU), tracrRNA 2-32 (M32T-ExU), tracrRNA 2-33 (M31T-ExU), tracrRNA 2-34 (M27T-ExU), tracrRNA 2-35 (M18T-ExU), tracrRNA 2-36 (M13T-ExU), tracrRNA 102 (M12/M18/3′T-ExU), or tracrRNA 103 (6F-M12/M18/3′T-ExU) of Table 4.

In one aspect, the disclosure provides a chemically modified guide RNA comprising: (a) a crRNA portion comprising (i) a guide sequence capable of hybridizing to a target polynucleotide sequence, and (ii) a repeat sequence: and (b) a tracrRNA portion comprising an anti-repeat nucleotide sequence that is complementary to the repeat sequence, wherein the tracrRNA portion comprises at least one exNA intersubunit linkage.

In certain embodiments, the at least one exNA intersubunit linkage is in a tracrRNA portion stem-loop (e.g., stem-loop 1, stem-loop 2, and/or stem-loop 3).

In certain embodiments, the at least one exNA intersubunit linkage is in the anti-repeat nucleotide sequence.

In certain embodiments, the tracrRNA portion comprises the exNA intersubunit linkage between one or more of positions 66 to 67, 65 to 66, 64 to 65, 61 to 62, 57 to 58, 47 to 48, 39 to 40, 38 to 39, 33 to 34, 31 to 32, 30 to 31, 26 to 27, 23 to 24, 17 to 18, 12 to 13, 11 to 12, and 4 to 5, from the 5′ end of the tracrRNA portion.

In certain embodiments, the tracrRNA portion comprises the exNA intersubunit linkage between one or more of positions 66 to 67, 65 to 66, 64 to 65, 61 to 62, 57 to 58, 47 to 48, 39 to 40, 38 to 39, 33 to 34, 31 to 32, 30 to 31, 26 to 27, 23 to 24, 17 to 18, 12 to 13, 11 to 12, and 4 to 5, from the 5′ end of the tracrRNA portion set forth in SEQ ID NO: 2.

In certain embodiments, the exNA intersubunit linkage comprises the intersubunit linkage of Formula Ia:

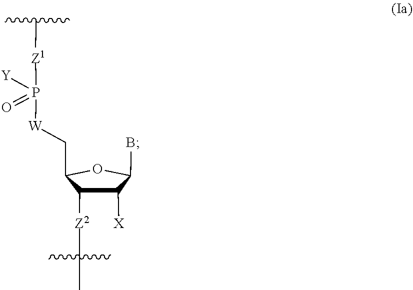

wherein:
B is a base moiety;
W is O or O(CH₂)ₙ¹, wherein n¹ is 1 to 10;
X is selected from the group consisting of H, OH, OR¹, R¹, F, Cl, Br, I, SH, SR¹, NH₂, NHR¹, NR¹₂, and COOR¹;
Y is selected from the group consisting of O⁻, OH, OR², NH , NH₂, NR²₂, BH₃, S⁻, R², and SH;
Z¹ is O or O(CH₂)ₙ² wherein n² is 1 to 10;
Z² is O or O(CH₂)ₙ³ wherein n³ is 1 to 10;
R¹ is a substituted or unsubstituted C₁-C₆ alkyl, alkenyl, alkynyl, aryl, or mixtures thereof; and
R² is a substituted or unsubstituted C₁-C₆ alkyl, alkenyl, alkynyl, aryl, or mixtures thereof, wherein:
Z₁ is O(CH₂)ₙ² and W is O, or Z₁ is O and W is O(CH₂)ₙ¹, or Z₁ is O(CH₂)n² and W is O(CH₂)ₙ¹.

In certain embodiments, Z¹ is O(CH₂)ₙ², n² is 1, W is O, and Y is O⁻. In certain embodiments, Z¹ is O, W is O(CH₂)ₙ¹, n¹ is 1, and Y is O⁻. In certain embodiments, Z¹ is O(CH₂)ₙ², n² is 1, W is O, and Y is O⁻. In certain embodiments, Z¹ is O(CH₂)ₙ², n² is 1, W is O(CH₂)ₙ¹, and Y is O⁻. In certain embodiments, Z¹ is O(CH₂)ₙ², n² is 1, W is O(CH₂)ₙ¹, and Y is S⁻. In certain embodiments, Y is S⁻. In certain embodiments, X is OR¹ or F. In certain embodiments, the base moiety B is selected from the group consisting of adenine, guanine, cytosine, and uracil.

In certain embodiments, the crRNA portion and/or the tracrRNA portion further comprise at least one modified nucleotide selected from a modification of a ribose group, a phosphate group, a nucleobase, or a combination thereof.

In certain embodiments, each modification of the ribose group is independently selected from the group consisting of 2′-O-methyl, 2′-fluoro, 2′-deoxy, 2′-O-(2-methoxyethyl) (MOE), 2′-NH₂ (2′-amino), 4 -thio, a bicyclic nucleotide, a locked nucleic acid (LNA), a 2′-(S)-constrained ethyl (S-cEt), a constrained MOE, and a 2′-O,4′-C-aminomethylene bridged nucleic acid (2′,4′-BNA^{NC}).

In certain embodiments, at least 80% of the ribose groups are chemically modified. In certain embodiments, at least 90% of the ribose groups are chemically modified. In certain embodiments, 100% of the ribose groups are chemically modified.

In certain embodiments, each modification of the phosphate group is independently selected from the group consisting of a phosphorothioate, phosphonoacetate (PACE), thiophosphonoacetate (thioPACE), amide, triazole, phosphonate, and phosphotriester modification.

In certain embodiments, each modification of the nucleobase group is independently selected from the group consisting of 2-thiouridine, 4-thiouridine, N⁶-methyladenosine, pseudouridine, 2,6-diaminopurine, inosine, thymidine, 5-methylcytosine, 5-substituted pyrimidine, isoguanine, isocytosine, and halogenated aromatic groups.

In certain embodiments, the guide RNA comprises at least 90% modified nucleotide. In certain embodiments, the guide RNA comprises 100% modified nucleotides.

In certain embodiments, the chemically modified guide RNA comprises a tracrRNA portion modification pattern selected from the group consisting of:

| Name (Alt. | |
|---|---|
| Name) | Sequence |
| tracrRNA 2 | mA#mG#mC#mAmUmAmGmCmAmAmGrUrUmArAmA |
| | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 15) |
| tracrRNA 2-1 | mA#mG#mC#mAmUmAmGmCmAmAmGrUrUmArAmA |
| (67T-ExU) | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#exm |
| | U(SEQ ID NO: 16) |
| tracrRNA 2-2 | mA#mG#mC#mAmUmAmGmCmAmAmGrUrUmArAmA |
| (66T-ExU) | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#exmU#m |
| | U(SEQ ID NO: 17) |
| tracrRNA 2-3 | mA#mG#mC#mAmUmAmGmCmAmAmGrUrUmArAmA |
| (65T-ExU) | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#exmU#mU#m |
| | U(SEQ ID NO: 18) |
| tracrRNA 2-5 | mA#mG#mC#mAmUmAmGmCmAmAmGrUrUmArAmA |
| (66/67T-ExU) | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#exmU#e |
| | xmU(SEQ ID NO: 19) |
| tracrRNA 2-6 | mA#mG#mC#mAmUmAmGmCmAmAmGrUrUmArAmA |
| (65/66/67T- | mArUmAmAmGmGrCrUmArGrUrCmCrGrUrUmAmUmC |
| ExU) | mAmAmCmUmUmGmAmAmAmAmAmGmUmGmGmCm |
| | AmCmCmGmAmGmUmCmGmGmUmGmC#exmU#exmU |
| | #exmU(SEQ ID NO: 20) |
| tracrRNA 2-8 | mA#mG#mC#mAmUmAmGmCmAmAmGrUrUmArAmA |
| (62T-ExU) | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGexmUmGmC#mU#mU#m |
| | U(SEQ ID NO: 21) |
| tracrRNA 2- | mA#mG#mC#mAmUmAmGmCmAmAmGrUrUmArAmA |
| 10 (58T-ExU) | mArUmAmAmGmGrCrUmArGrUrCmCrGrUrUmAmUmC |
| | mAmAmCmUmUmGmAmAmAmAmAmGmUmGmGmCm |
| | AmCmCmGmAmGexmUmCmGmGmUmGmC#mU#mU#m |
| | U(SEQ ID NO: 22) |
| tracrRNA 2- | mA#mG#mC#mAmUmAmGmCmAmAmGrUrUmArAmA |
| 14 (48T-ExU) | mArUmAmAmGmGrCrUmArGrUrCmCrGrUrUmAmUmC |
| | mAmAmCmUmUmGmAmAmAmAmAmGexmUmGmGm |
| | CmAmCmCmGmAmGmUmCmGmGmUmGmC#mU#mU# |
| | mU(SEQ ID NO: 23) |
| tracrRNA 2- | mA#mG#mC#mAmUmAmGmCmAmAmGrUrUmArAmA |
| 15 (40T-ExU) | mArUmAmAmGmGrCrUmArGrUrCmCrGrUrUmAmUmC |
| | mAmAmCmUexmUmGmAmAmAmAmAmGmUmGmGm |
| | CmAmCmCmGmAmGmUmCmGmGmUmGmC#mU#mU# |
| | mU(SEQ ID NO: 24) |
| tracrRNA 2- | mA#mG#mC#mAmUmAmGmCmAmAmGrUrUmArAmA |
| 16 (39T-ExU) | mArUmAmAmGmGrCrUmArGrUrCmCrGrUrUmAmUmC |
| | mAmAmCexmUmUmGmAmAmAmAmAmGmUmGmGm |
| | CmAmCmCmGmAmGmUmCmGmGmUmGmC#mU#mU# |
| | mU(SEQ ID NO: 25) |
| tracrRNA 2- | mA#mG#mC#mAmUmAmGmCmAmAmGrUrUmArAmA |
| 19 (34T-ExU) | mArUmAmAmGmGrCrUmArGrUrCmCrGrUrUmAexmUm |
| | CmAmAmCmUmUmGmAmAmAmAmAmGmUmGmGmC |
| | mAmCmCmGmAmGmUmCmGmGmUmGmC#mU#mU#m |
| | U(SEQ ID NO: 26) |
| tracrRNA 2- | mA#mG#mC#mAmUmAmGmCmAmAmGrUrUmArAmA |
| 20 (32T-ExU) | mArUmAmAmGmGrCrUmArGrUrCmCrGrUexrUmAmUm |
| | CmAmAmCmUmUmGmAmAmAmAmAmGmUmGmGmC |
| | mAmCmCmGmAmGmUmCmGmGmUmGmC#mU#mU#m |
| | U(SEQ ID NO: 27) |
| tracrRNA 2- | mA#mG#mC#mAmUmAmGmCmAmAmGrUrUmArAmA |
| 21 (31T-ExU) | mArUmAmAmGmGrCrUmArGrUrCmCrGexrUrUmAmUm |
| | CmAmAmCmUmUmGmAmAmAmAmAmGmUmGmGmC |
| | mAmCmCmGmAmGmUmCmGmGmUmGmC#mU#mU#m |
| | U(SEQ ID NO: 28) |
| tracrRNA 2- | mA#mG#mC#mAmUmAmGmCmAmAmGrUrUmArAmA |
| 23 (27T-ExU) | mArUmAmAmGmGrCrUmArGexrUrCmCrGrUrUmAmUm |
| | CmAmAmCmUmUmGmAmAmAmAmAmGmUmGmGmC |
| | mAmCmCmGmAmGmUmCmGmGmUmGmC#mU#mU#m |
| | U(SEQ ID NO: 29) |
| tracrRNA 2- | mA#mG#mC#mAmUmAmGmCmAmAmGrUrUmArAmA |
| 24 (24T-ExU) | mArUmAmAmGmGrCexrUmArGrUrCmCrGrUrUmAmUm |
| | CmAmAmCmUmUmGmAmAmAmAmAmGmUmGmGmC |
| | mAmCmCmGmAmGmUmCmGmGmUmGmC#mU#mU#m |
| | U(SEQ ID NO: 30) |
| tracrRNA 2- | mA#mG#mC#mAmUmAmGmCmAmAmGrUrUmArAmA |
| 25 (18T-ExU) | mAexrUmAmAmGmGrCrUmArGrUrCmCrGrUrUmAmUm |
| | CmAmAmCmUmUmGmAmAmAmAmAmGmUmGmGmC |
| | mAmCmCmGmAmGmUmCmGmGmUmGmC#mU#mU#m |
| | U(SEQ ID NO: 31) |
| tracrRNA 2- | mA#mG#mC#mAmUmAmGmCmAmAmGrUexrUmArAm |
| 26 (13T-ExU) | AmArUmAmAmGmGrCrUmArGrUrCmCrGrUrUmAmUm |
| | CmAmAmCmUmUmGmAmAmAmAmAmGmUmGmGmC |
| | mAmCmCmGmAmGmUmCmGmGmUmGmC#mU#mU#m |
| | U(SEQ ID NO: 32) |
| tracrRNA 2- | mA#mG#mC#mAmUmAmGmCmAmAmGexrUrUmArAm |
| 27 (12T-ExU) | AmArUmAmAmGmGrCrUmArGrUrCmCrGrUrUmAmUm |
| | CmAmAmCmUmUmGmAmAmAmAmAmGmUmGmGmC |
| | mAmCmCmGmAmGmUmCmGmGmUmGmC#mU#mU#m |
| | U(SEQ ID NO: 33) |
| tracrRNA 2- | mA#mG#mC#mAexmUmAmGmCmAmAmGrUrUmArAm |
| 30 (5T-ExU) | AmArUmAmAmGmGrCrUmArGrUrCmCrGrUrUmAmUm |
| | CmAmAmCmUmUmGmAmAmAmAmAmGmUmGmGmC |
| | mAmCmCmGmAmGmUmCmGmGmUmGmC#mU#mU#m |
| | U(SEQ ID NO: 34) |
| tracrRNA 2- | mA#mG#mC#mAmUmAmGmCmAmAmGexrUrUmArAm |
| 31 | AmArUmAmAmGmGrCexrUmArGrUrCmCrGrUrUmAmU |
| (12/24/3′T- | mCmAmAmCmUmUmGmAmAmAmAmAmGmUmGmGm |
| ExU) | CmAmCmCmGmAmGmUmCmGmGmUmGmC#exmU#ex |
| | mU#exmU(SEQ ID NO: 35) |
| tracrRNA 2- | mA#mG#mC#mAmUmAmGmCmAmAmGrUrUmArAmA |
| 32 (M32T- | mArUmAmAmGmGrCrUmArGrUrCmCrGrUexmUmAmU |
| ExU) | mCmAmAmCmUmUmGmAmAmAmAmAmGmUmGmGm |
| | CmAmCmCmGmAmGmUmCmGmGmUmGmC#mU#mU# |
| | mU(SEQ ID NO: 36) |
| tracrRNA 2- | mA#mG#mC#mAmUmAmGmCmAmAmGrUrUmArAmA |
| 33 (M31T- | mArUmAmAmGmGrCrUmArGrUrCmCrGexmUrUmAmU |
| ExU) | mCmAmAmCmUmUmGmAmAmAmAmAmGmUmGmGm |
| | CmAmCmCmGmAmGmUmCmGmGmUmGmC#mU#mU# |
| | mU(SEQ ID NO: 37) |
| tracrRNA 2- | mA#mG#mC#mAmUmAmGmCmAmAmGrUrUmArAmA |
| 34 (M27T- | mArUmAmAmGmGrCrUmArGexmUrCmCrGrUrUmAmU |
| ExU) | mCmAmAmCmUmUmGmAmAmAmAmAmGmUmGmGm |
| | CmAmCmCmGmAmGmUmCmGmGmUmGmC#mU#mU# |
| | mU(SEQ ID NO: 38) |
| tracrRNA 2- | mA#mG#mC#mAmUmAmGmCmAmAmGrUrUmArAmA |
| 35 (M18T- | mAexmUmAmAmGmGrCrUmArGrUrCmCrGrUrUmAmU |
| ExU) | mCmAmAmCmUmUmGmAmAmAmAmAmGmUmGmGm |
| | CmAmCmCmGmAmGmUmCmGmGmUmGmC#mU#mU# |
| | mU(SEQ ID NO: 39) |
| tracrRNA 2- | mA#mG#mC#mAmUmAmGmCmAmAmGrUexmUmArAm |
| 36 (M13T- | AmArUmAmAmGmGrCrUmArGrUrCmCrGrUrUmAmUm |
| ExU) | CmAmAmCmUmUmGmAmAmAmAmAmGmUmGmGmC |
| | mAmCmCmGmAmGmUmCmGmGmUmGmC#mU#mU#m |
| | U(SEQ ID NO: 40) |
| tracrRNA | mA#mG#mC#mAmUmAmGmCmAmAmGexmUrUmArAm |
| 102 | AmAexmUmAmAmGmGrCrUmArGrUrCmCrGrUrUmAm |
| (M12/M18/3′ | UmCmAmAmCmUmUmGmAmAmAmAmAmGmUmGmG |
| T-ExU) | mCmAmCmCmGmAmGmUmCmGmGmUmGmC#exmU#e |
| | xmU#exmU(SEQ ID NO: 41) |
| tracrRNA | mA#mG#mC#mAmUmAmGmCmAmAmGexmUrUmAfAm |
| 103 (6F- | AmAexmUmAmAmGmGfCrUmAfGrUfCmCfGrUfUmAm |
| M12/M18/3′ | UmCmAmAmCmUmUmGmAmAmAmAmAmGmUmGmG |
| T-ExU) | mCmAmCmCmGmAmGmUmCmGmGmUmGmC#exmU#e |
| | xmU#exmU(SEQ ID NO: 42) |

KEY:
rN = RNA, mN = 2′-O-methyl RNA, fN = 2′-fluoro RNA, NexN = exNA linkage, N = A, U, G, or C N#N = phosphorothioate linkage

In certain embodiments, the chemically modified guide RNA comprises a crRNA portion modification pattern of any one of crRNA 1 to crRNA 134 of Table 1 or a crRNA portion modification pattern of any one of crRNA Ex20-1, crRNA Ex20-4, crRNA Ex20-5, crRNA Ex20-7, crRNA Ex20-8, crRNA Ex20-9, crRNA Ex20-10, crRNA Ex20-12, crRNA Ex20-13, or crRNA Ex20-15 of Table 3.

In certain embodiments, the chemically modified guide RNA further comprises at least one moiety conjugated to the guide RNA.

In certain embodiments, the at least one moiety is conjugated to at least one of the 5′ end of the crRNA portion, the 3′ end of the crRNA portion, the 5′ end of the tracrRNA portion, and the 3′ end of the tracrRNA portion.

In certain embodiments, the at least one moiety increases cellular uptake of the guide RNA.

In certain embodiments, the at least one moiety promotes specific tissue distribution of the guide RNA.

In certain embodiments, the at least one moiety is selected from the group consisting of fatty acids, steroids, secosteroids, lipids, gangliosides analogs, nucleoside analogs, endocannabinoids, vitamins, receptor ligands, peptides, aptamers, and alkyl chains.

In certain embodiments, the at least one moiety is selected from the group consisting of cholesterol, docosahexaenoic acid (DHA), docosanoic acid (DCA), lithocholic acid (LA), GalNAc, amphiphilic block copolymer (ABC), hydrophilic block copolymer (HBC), poloxamer, Cy5, and Cy3.

In certain embodiments, the at least one moiety is conjugated to the guide RNA via a linker.

In certain embodiments, the linker is selected from the group consisting of an ethylene glycol chain, an alkyl chain, a polypeptide, a polysaccharide, and a block copolymer.

In certain embodiments, the at least one moiety is a modified lipid. In certain embodiments, the modified lipid is a branched lipid.

In certain embodiments, the guide RNA binds to a Cas9 nuclease selected from the group consisting of S. pyogenes Cas9 (SpCas9), S. aureus Cas9 (SaCas9), N. meningitidis Cas9 (NmCas9), C. jejuni Cas9 (CjCas9), and Geobacillus Cas9 (GeoCas9).

In certain embodiments, the Cas9 is a variant Cas9 with altered activity.

In certain embodiments, the variant Cas9 is selected from the group consisting of a Cas9 nickase (nCas9), a catalytically dead Cas9 (dCas9), a hyper accurate Cas9 (HypaCas9), a high fidelity Cas9 (Cas9-HF), an enhanced specificity Cas9 (eCas9), and an expanded PAM Cas9 (xCas9).

In certain embodiments, Cas9 off-target activity is reduced relative to an unmodified guide RNA.

In certain embodiments, Cas9 on-target activity is increased relative to an unmodified guide RNA.

In certain embodiments, the chemically modified guide RNA further comprises a nucleotide or non-nucleotide loop or linker linking the 3′ end of the crRNA portion to the 5′ end of the tracrRNA portion.

In certain embodiments, the non-nucleotide linker comprises an ethylene glycol oligomer linker.

In certain embodiments, the nucleotide loop is chemically modified.

In certain embodiments, the nucleotide loop comprises the nucleotide sequence of GAAA.

In certain embodiments, chemically modified guide RNA comprises at least about 50% activity relative to an unmodified guide RNA.

In one aspect, the disclosure provides a method of altering expression of a target gene in a cell, comprising administering to said cell a genome editing system comprising: the chemically modified guide RNA described above; and an RNA-guided nuclease or a polynucleotide encoding an RNA-guided nuclease.

In certain embodiments, the target gene is in a cell in an organism.

In certain embodiments, expression of the target gene is knocked out or knocked down.

In certain embodiments, the sequence of the target gene is modified, edited, corrected or enhanced.

In certain embodiments, the guide RNA and the RNA-guided nuclease comprise a ribonucleoprotein (RNP) complex.

In certain embodiments, the RNA-guided nuclease is selected from the group consisting of S. pyogenes Cas9 (SpCas9), S. aureus Cas9 (SaCas9), N. meningitidis Cas9 (NmCas9), C. jejuni Cas9 (CjCas9), and Geobacillus Cas9 (GeoCas9).

In certain embodiments, the Cas9 is a variant Cas9 with altered activity.

In certain embodiments, the variant Cas9 is selected from the group consisting of a Cas9 nickase (nCas9), a catalytically dead Cas9 (dCas9), a hyper accurate Cas9 (HypaCas9), a high fidelity Cas9 (Cas9-HF), an enhanced specificity Cas9 (eCas9), and an expanded PAM Cas9 (xCas9).

In certain embodiments, the polynucleotide encoding an RNA-guided nuclease comprises a vector.

In certain embodiments, the vector is a viral vector.

In certain embodiments, the viral vector is an adeno-associated virus (AAV) vector or a lentivirus (LV) vector.

In certain embodiments, expression of the target gene is reduced by at least about 20%.

In one aspect, the disclosure provides a CRISPR genome editing system comprising: a chemically modified guide RNA described above; and an RNA-guided nuclease or a polynucleotide encoding an RNA-guided nuclease.

In certain embodiments, the RNA-guided nuclease is selected from the group consisting of S. pyogenes Cas9 (SpCas9), S. aureus Cas9 (SaCas9), N. meningitidis Cas9 (NmCas9), C. jejuni Cas9 (CjCas9), and Geobacillus Cas9 (GeoCas9).

In certain embodiments, the Cas9 is a variant Cas9 with altered activity.

In certain embodiments, the variant Cas9 is selected from the group consisting of a Cas9 nickase (nCas9), a catalytically dead Cas9 (dCas9), a hyper accurate Cas9 (HypaCas9), a high fidelity Cas9 (Cas9-HF), an enhanced specificity Cas9 (eCas9), and an expanded PAM Cas9 (xCas9).

In certain embodiments, Cas9 off-target activity is reduced relative to an unmodified guide RNA.

In certain embodiments, Cas9 on-target activity is increased relative to an unmodified guide RNA.

### BRIEF DESCRIPTION OF THE DRAWINGS

The foregoing and other features and advantages of the present disclosure will be more fully understood from the following detailed description of illustrative embodiments taken in conjunction with the accompanying drawings. The patent or application file contains at least one drawing executed in color. Copies of this patent or patent application publication with color drawing(s) will be provided by the Office upon request and payment of the necessary fee.

FIG. 1 depicts a schematic of a crRNA (SEQ ID NO: 1) and tracrRNA (SEQ ID NO: 2) when paired with the target genomic DNA.

FIG. 2 depicts editing efficiencies several crRNAs tested containing exNA intersubunit linkages at various positions. TracrRNA T2 was paired with the crRNAs. The Traffic Light Reporter Multi-Cas Variant 1 (TLR-MCV1) reporter was used. The graphs show the percentages of red fluorescent (RF) cells obtained by fluorescence activated cell sorting (FACS) analysis. Data are mean values of three biological replicates and error bars represent s.e.m.

FIG. 3 depicts editing efficiencies several tracrRNAs tested containing exNA intersubunit linkages at various positions. CrRNA C20 was paired with the tracrRNAs. The TLR-MCV1 reporter was used. The graphs show the percentages of RF cells obtained by FACS analysis. Data are mean values of three biological replicates and error bars represent s.e.m.

FIG. 4 depicts editing efficiencies of several exNA-containing crRNAs paired with several exNA-containing tracrRNAs. The TLR-MCV1 reporter was used. The graphs show the percentages of RF cells obtained by FACS analysis. Data are mean values of three biological replicates and error bars represent s.e.m.

FIG. 5 depicts editing efficiencies of several exNA-containing several exNA-containing tracrRNAs paired with crRNAs C39 and C40. The TLR-MCV1 reporter was used. The graphs show the percentages of RF cells obtained by FACS analysis. Data are mean values of three biological replicates and error bars represent s.e.m.

FIG. 6 depicts editing efficiencies of several exNA-containing several exNA-containing tracrRNAs paired with crRNA C39. Guide RNA: Cas9 RNPs were used at 2 pmol and 20 pmol. The TLR-MCV1 reporter was used. The graphs show the percentages of RF cells obtained by FACS analysis. Data are mean values of three biological replicates and error bars represent s.e.m.

FIG. 7 depicts editing efficiencies of several exNA-containing tracrRNAs paired with crRNAs C40 and C45. The TLR-MCV1 reporter was used. The graphs show the percentages of RF cells obtained by FACS analysis. Data are mean values of three biological replicates and error bars represent s.e.m.

FIG. 8 depicts editing efficiencies of several exNA-containing tracrRNAs paired with crRNAs C20, C21, C39, C40, and C45. The TLR-MCVI reporter was used. The graphs show the percentages of RF cells obtained by FACS analysis. Data are mean values of three biological replicates and error bars represent s.e.m.

FIG. 9 summarizes the exNA intersubunit linkages provided herein.

FIG. 10 provides a synthesis of a 2′-OMe-exNA phosphoramidite 9a.

FIG. 11 provides a synthesis of a 2′-F-exNA phosphoramidite 9b.

FIG. 12 provides a synthesis of an exNA-C phosphoramidite.

FIG. 13 provides a synthesis of an exNA-G and an exNA-A phosphoramidite.

FIG. 14 provides a synthesis of a 5′-3′-bis-methylene-exNA phosphoramidite.

FIG. 15 provides a synthesis of an exNA-ribo-uridine phosphoramidite.

FIG. 16 provides a synthesis of an exNA-ribo-cytosine phosphoramidite.

FIG. 17 provides a synthesis of an exNA-ribo-guanosine or exNA-ribo-adenine phosphoramidite.

FIG. 18 provides a synthesis of a phosphoramidite monomer.

FIG. 19 provides a universal scheme for exNA conversion of sugar-modified nucleotides.

FIG. 20 provides synthesis for oligonucleotides incorporating exNA backbones.

### DETAILED DESCRIPTION

Provided herewith are novel chemically modified crRNAs and tracrRNAs containing at least one extended nucleic acid (exNA) intersubunit linkage. The exNA-modified crRNAs and tracrRNAs display exceptional resistance to degradation (e.g., nuclease degradation) while retaining genome editing activity. Methods of using the crRNAs and tracrRNAs of the disclosure for genome editing with a CRISPR nuclease and kits for performing the same are also provided.

Unless otherwise defined herein, nomenclature used in connection with cell and tissue culture, molecular biology, immunology, microbiology, genetics and protein and nucleic acid chemistry and hybridization described herein are those well-known and commonly used in the art. The methods and techniques provided herein are usually performed according to conventional methods well known in the art and as described in various general and more specific references that are cited and discussed throughout the present specification unless otherwise indicated. Enzymatic reactions and purification techniques are performed according to manufacturer's specifications unless otherwise specified, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art, unless otherwise specified. Standard techniques are used for chemical syntheses, chemical analyses, pharmaceutical preparation, formulation, and delivery, and treatment of patients.

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

So that the disclosure may be more readily understood, certain terms are first defined.

As used herein, the term “guide RNA” or “gRNA” refer to any nucleic acid that promotes the specific association (or “targeting”) of an RNA-guided nuclease such as a Cas9 to a target sequence (e.g., a genomic or episomal sequence) in a cell.

As used herein, a “modular” or “dual RNA” guide comprises more than one, and typically two, separate RNA molecules, such as a CRISPR RNA (crRNA) and a trans-activating crRNA (tracrRNA), which are usually associated with one another, for example by duplexing. gRNAs and their component parts are described throughout the literature (see, e.g., Briner et al. Mol. Cell, 56(2), 333-339 (2014), which is incorporated by reference).

As used herein, a “unimolecular gRNA,” “chimeric gRNA,” or “single guide RNA (sgRNA)” comprises a single RNA molecule. The sgRNA may be a crRNA and tracrRNA linked together. For example, the 3′ end of the crRNA may be linked to the 5′ end of the tracrRNA. A crRNA and a tracrRNA may be joined into a single unimolecular or chimeric gRNA, for example, by means of a four nucleotide (e.g., GAAA) “tetraloop” or “linker” sequence bridging complementary regions of the crRNA (at its 3′ end) and the tracrRNA (at its 5′ end).

As used herein, a “repeat” sequence or region is a nucleotide sequence at or near the 3′ end of the crRNA which is complementary to an anti-repeat sequence of a tracrRNA.

As used herein, an “anti-repeat” sequence or region is a nucleotide sequence at or near the 5′ end of the tracrRNA which is complementary to the repeat sequence of a crRNA.

Additional details regarding guide RNA structure and function, including the gRNA/Cas9 complex for genome editing may be found in, at least, Mali et al. Science, 339(6121), 823-826 (2013): Jiang et al. Nat. Biotechnol. 31(3). 233-239 (2013); and Jinek et al. Science, 337(6096), 816-821 (2012); which are incorporated by reference herein.

As used herein, a “guide sequence” or “targeting sequence” refers to the nucleotide sequence of a gRNA, whether unimolecular or modular, that is fully or partially complementary to a target domain or target polynucleotide within a DNA sequence in the genome of a cell where editing is desired. Guide sequences are typically 10-30 nucleotides in length, preferably 16-24 nucleotides in length (for example, 16, 17, 18, 19, 20, 21, 22, 23 or 24 nucleotides in length), and are at or near the 5′ terminus of a Cas9 gRNA.

As used herein, a “target domain” or “target polynucleotide sequence” is the DNA sequence in a genome of a cell that is complementary to the guide sequence of the gRNA.

In addition to the targeting domains, gRNAs typically include a plurality of domains that influence the formation or activity of gRNA/Cas9 complexes. For example, as mentioned above, the duplexed structure formed by first and secondary complementarity domains of a gRNA (also referred to as a repeat: anti-repeat duplex) interacts with the recognition (REC) lobe of Cas9 and may mediate the formation of Cas9/gRNA complexes (Nishimasu et al. Cell 156: 935-949 (2014): Nishimasu et al. Cell 162(2), 1113-1126 (2015), both incorporated by reference herein). It should be noted that the first and/or second complementarity domains can contain one or more poly-A tracts, which can be recognized by RNA polymerases as a termination signal.

The sequence of the first and second complementarity domains are, therefore, optionally modified to eliminate these tracts and promote the complete in vitro transcription of gRNAs, for example through the use of A-G swaps as described in Briner 2014, or A-U swaps. These and other similar modifications to the first and second complementarity domains are within the scope of the present disclosure.

Along with the first and second complementarity domains, Cas9 gRNAs typically include two or more additional duplexed regions that are necessary for nuclease activity in vivo but not necessarily in vitro (Nishimasu 2015, supra). A first stem-loop near the 3′ portion of the second complementarity domain is referred to variously as the “proximal domain,” “stem loop 1” (Nishimasu 2014, supra: Nishimasu 2015, supra) and the “nexus” (Briner 2014, supra). One or more additional stem loop structures are generally present near the 3′ end of the gRNA, with the number varying by species: S. pyogenes gRNAs typically include two 3′ stem loops (for a total of four stem loop structures including the repeat: anti-repeat duplex), while s. aureus and other species have only one (for a total of three). A description of conserved stem loop structures (and gRNA structures more generally) organized by species is provided in Briner 2014, which is incorporated herein by reference. Additional details regarding guide RNAs generally may be found in WO2018026976A1, which is incorporated herein by reference.

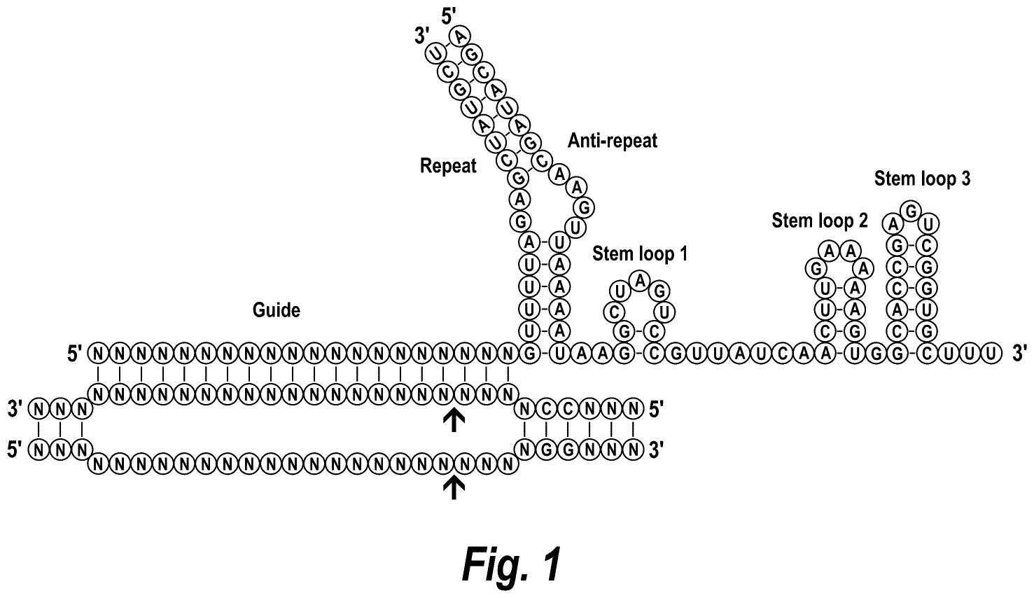

A representative guide RNA is shown in FIG. 1.

### Chemically Modified Guide RNA

The chemically modified guide RNAs of the disclosure possess improved in vivo stability, improved genome editing efficacy, and/or reduced immunotoxicity relative to unmodified or minimally modified guide RNAs.

Chemically modified guide RNAs of the disclosure contain one or more modified nucleotides comprising a modification in a ribose group, a phosphate group, a nucleobase, or a combination thereof.

Chemical modifications to the ribose group may include, but are not limited to, 2′-O-methyl, 2′-fluoro, 2′-deoxy, 2′-O-(2-methoxyethyl) (MOE), 2′-NH₂ (2′-amino), 4′-thio, 2′-O-Allyl, 2′-O-Ethylamine, 2′-O-Cyanoethyl, 2′-O-Acetalester, or a bicyclic nucleotide, such as locked nucleic acid (LNA), 2′-(S)-constrained ethyl (S-cEt), constrained MOE, or 2′-O,4′-C-aminomethylene bridged nucleic acid (2′,4′-BNA^{NC}).

The term “4′-thio” as used herein corresponds to a ribose group modification where the sugar ring oxygen of the ribose is replaced with a sulfur.

Chemical modifications to the phosphate group may include, but are not limited to, a phosphorothioate, phosphonoacetate (PACE), thiophosphonoacetate (thioPACE), amide, triazole, phosphonate, or phosphotriester modification.

In an embodiment, the crRNA portion of the chemically modified guide RNA comprises between 1 and 20 phosphorothioate modifications (i.e., 1, 2, 3, 4, 5, 6, 7, 8, 9, 10, 11, 12, 13, 14, 15, 16, 17, 18, 19, or 20 phosphorothioate modifications). In an embodiment, the crRNA portion of the chemically modified guide RNA comprises between 1 and 20 phosphorothioate modifications (i.e., 1, 2, 3, 4, 5, 6, 7, 8, 9, 10, 11, 12, 13, 14, 15, 16, 17, 18, 19, or 20 phosphorothioate modifications) and comprises at least about 50% activity relative to a guide RNA that does not comprise phosphorothioate modifications (e.g., 50% activity, 60% activity, 70% activity, 80% activity, 90% activity, 95% activity, or 100% activity, relative to a guide RNA that does not comprise phosphorothioate modifications).

Chemical modifications to the nucleobase may include, but are not limited to, 2-thiouridine, 4-thiouridine, N⁶-methyladenosine, pseudouridine, 2,6-diaminopurine, inosine, thymidine, 5-methylcytosine, 5-substituted pyrimidine, isoguanine, isocytosine, or halogenated aromatic groups.

The chemically modified guide RNAs may have one or more chemical modifications in the crRNA portion and/or the tracrRNA portion for a modular or dual RNA guide. The chemically modified guide RNAs may also have one or more chemical modifications in the single guide RNA for the unimolecular guide RNA.

The chemically modified guide RNAs may comprise at least about 50% to at least about 100% chemically modified nucleotides, at least about 60% to at least about 100% chemically modified nucleotides, at least about 70% to at least about 100% chemically modified nucleotides, at least about 80% to at least about 100% chemically modified nucleotides, at least about 90% to at least about 100% chemically modified nucleotides, and at least about 95% to at least about 100% chemically modified nucleotides.

The chemically modified guide RNAs may comprise at least about 50% chemically modified nucleotides, at least about 60% chemically modified nucleotides, at least about 70% chemically modified nucleotides, at least about 80% chemically modified nucleotides, at least about 90% chemically modified nucleotides, at least about 95% chemically modified nucleotides, at least about 99% chemically modified, or 100% (fully) chemically modified nucleotides.

The chemically modified guide RNAs may comprise at least about 80%, 81%, 82%, 83%, 84%, 85%, 86%, 87%, 88%, 89%, 90%, 91%, 92%, 93%, 94%, 95%, 96%, 97%, 98%, 99%, or 100% chemically modified nucleotides.

Guide RNAs that comprise at least about 80% chemically modified nucleotides to at least about 99% chemically modified nucleotides are considered “heavily” modified, as used herein.

Guide RNAs that comprise 100% chemically modified nucleotides are considered “fully” modified, as used herein.

In certain exemplary embodiments, the chemically modified guide RNAs may comprise a chemically modified ribose group at about 50% of the guide RNA nucleotides to about 100% of the guide RNA nucleotides, at about 60% of the guide RNA nucleotides to about 100% of the guide RNA nucleotides, at about 70% of the guide RNA nucleotides to about 100% of the guide RNA nucleotides, at about 80% of the guide RNA nucleotides to about 100% of the guide RNA nucleotides, at about 90% of the guide RNA nucleotides to about 100% of the guide RNA nucleotides, and at about 95% of the guide RNA nucleotides to about 100% of the guide RNA nucleotides

In certain exemplary embodiments, the chemically modified guide RNAs may comprise a chemically modified ribose group at about 50% of the guide RNA nucleotides, at about 60% of the guide RNA nucleotides, at about 70% of the guide RNA nucleotides, at about 80% of the guide RNA nucleotides, at about 90% of the guide RNA nucleotides, at about 95% of the guide RNA nucleotides, at about 99% of the guide RNA nucleotides, or at 100% of the guide RNA nucleotides.

In certain exemplary embodiments, the chemically modified guide RNAs may comprise a chemically modified ribose group at about 80%, 81%, 82%, 83%, 84%, 85%, 86%, 87%, 88%, 89%, 90%, 91%, 92%, 93%, 94%, 95%, 96%, 97%, 98%, 99%, or 100% of the guide RNA nucleotides.

Guide RNAs that have at least about 80% of the ribose groups chemically modified to at least about 99% of the ribose groups chemically modified are considered “heavily” modified, as used herein.

Guide RNAs that have 100% of the ribose groups chemically modified are considered “fully” modified, as used herein.

In certain exemplary embodiments, the chemically modified guide RNAs may comprise a chemically modified phosphate group at about 50% of the guide RNA nucleotides to about 100% of the guide RNA nucleotides, at about 60% of the guide RNA nucleotides to about 100% of the guide RNA nucleotides, at about 70% of the guide RNA nucleotides to about 100% of the guide RNA nucleotides, at about 80% of the guide RNA nucleotides to about 100% of the guide RNA nucleotides, at about 90% of the guide RNA nucleotides to about 100% of the guide RNA nucleotides, and at about 95% of the guide RNA nucleotides to about 100% of the guide RNA nucleotides

In certain exemplary embodiments, the chemically modified guide RNAs may comprise a chemically modified phosphate group at about 50% of the guide RNA nucleotides, at about 60% of the guide RNA nucleotides, at about 70% of the guide RNA nucleotides, at about 80% of the guide RNA nucleotides, at about 90% of the guide RNA nucleotides, at about 95% of the guide RNA nucleotides, at about 99% of the guide RNA nucleotides, or at 100% of the guide RNA nucleotides.

In certain exemplary embodiments, the chemically modified guide RNAs may comprise a chemically modified phosphate group at about 80%, 81%, 82%, 83%, 84%, 85%, 86%, 87%, 88%, 89%, 90%, 91%, 92%, 93%, 94%, 95%, 96%, 97%, 98%, 99%, or 100% of the guide RNA nucleotides.

Guide RNAs that have at least about 80% of the phosphate groups chemically modified to at least about 99% of the phosphate groups chemically modified are considered “heavily” modified, as used herein.

Guide RNAs that have 100% of the phosphate groups chemically modified are considered “fully” modified, as used herein.

In certain exemplary embodiments, the chemically modified guide RNAs may comprise a chemically modified nucleobase at about 50% of the guide RNA nucleotides to about 100% of the guide RNA nucleotides, at about 60% of the guide RNA nucleotides to about 100% of the guide RNA nucleotides, at about 70% of the guide RNA nucleotides to about 100% of the guide RNA nucleotides, at about 80% of the guide RNA nucleotides to about 100% of the guide RNA nucleotides, at about 90% of the guide RNA nucleotides to about 100% of the guide RNA nucleotides, and at about 95% of the guide RNA nucleotides to about 100% of the guide RNA nucleotides.

In certain exemplary embodiments, the chemically modified guide RNAs may comprise a chemically modified nucleobase at about 50% of the guide RNA nucleotides, at about 60% of the guide RNA nucleotides, at about 70% of the guide RNA nucleotides, at about 80% of the guide RNA nucleotides, at about 90% of the guide RNA nucleotides, at about 95% of the guide RNA nucleotides, at about 99% of the guide RNA nucleotides, or at 100% of the guide RNA nucleotides.

In certain exemplary embodiments, the chemically modified guide RNAs may comprise a chemically modified nucleobase at about 80%, 81%, 82%, 83%, 84%, 85%, 86%, 87%, 88%, 89%, 90%, 91%, 92%, 93%, 94%, 95%, 96%, 97%, 98%, 99%, or 100% of the guide RNA nucleotides.

Guide RNAs that have at least about 80% of the nucleobases chemically modified to at least about 99% of the nucleobases chemically modified are considered “heavily” modified, as used herein.

Guide RNAs that have 100% of the nucleobases chemically modified are considered “fully” modified, as used herein.

In certain exemplary embodiments, the chemically modified guide RNAs may comprise any combination of chemically modified ribose groups, chemically modified phosphate groups, and chemically modified nucleobases at about 50% of the guide RNA nucleotides to about 100% of the guide RNA nucleotides, at about 60% of the guide RNA nucleotides to about 100% of the guide RNA nucleotides, at about 70% of the guide RNA nucleotides to about 100% of the guide RNA nucleotides, at about 80% of the guide RNA nucleotides to about 100% of the guide RNA nucleotides, at about 90% of the guide RNA nucleotides to about 100% of the guide RNA nucleotides, and at about 95% of the guide RNA nucleotides to about 100% of the guide RNA nucleotides.

In certain exemplary embodiments, the chemically modified guide RNAs may comprise any combination of chemically modified ribose groups, chemically modified phosphate groups, and chemically modified nucleobases at about 50% of the guide RNA nucleotides, at about 60% of the guide RNA nucleotides, at about 70% of the guide RNA nucleotides, at about 80% of the guide RNA nucleotides, at about 90% of the guide RNA nucleotides, at about 95% of the guide RNA nucleotides, at about 99% of the guide RNA nucleotides, or at 100% of the guide RNA nucleotides.

In certain exemplary embodiments, the chemically modified guide RNAs may comprise any combination of chemically modified ribose groups, chemically modified phosphate groups, and chemically modified nucleobases at about 80%, 81%, 82%, 83%, 84%, 85%, 86%, 87%, 88%, 89%, 90%, 91%, 92%, 93%, 94%, 95%, 96%, 97%, 98%, 99%, or 100% of the guide RNA nucleotides.

Guide RNAs that have at least about 80% of any combination of the ribose groups, the phosphate groups, and the nucleobases chemically modified to at least about 99% of the nucleobases chemically modified are considered “heavily” modified, as used herein.

Guide RNAs that have 100% of any combination of the ribose groups, the phosphate groups, and the nucleobases chemically modified are considered “fully” modified, as used herein.

The heavily and fully chemically modified guide RNAs of the disclosure possess several advantages over the minimally modified guide RNAs in the art. Heavily and fully chemically modified guide RNAs are expected to ease chemical synthesis, further enhance in vivo stability, and provide a scaffold for terminally appended chemical functionalities that facilitate delivery and efficacy during clinical applications to genome editing.

The chemical modification pattern used in the guide RNA is such that activity of the guide RNA is maintained when paired with an RNA-guided DNA endonuclease, e.g., Cas9.

In an embodiment, the chemically modified guide RNAs of the disclosure comprise at least about 50% activity relative to an unmodified guide RNA (e.g., 50% activity, 60% activity, 70% activity, 80% activity, 90% activity, 95% activity, or 100% activity, relative to an unmodified guide RNA).

The activity of a guide RNA can be readily determined by any means known in the art. In an embodiment, % activity is measured with the traffic light reporter (TLR) Multi-Cas Variant 1 system (TLR-MCV1), described below. The TLR-MCV1 system will provide a % fluorescent cells which is a measure of % activity.

Exemplary chemical modification patterns for non-exNA-containing crRNA and tracrRNA sequences are described in Table 1 and Table 2 below. The non-exNA containing crRNAs of crRNA 1 through crRNA 134, and the non-exNA containing tracrRNAs of tracrRNA 1 through tracrRNA 116, are described in further detail in WO2019183000 and WO2021231606, each of which is incorporated herein by reference.

TABLE 1
Exemplary chemical modification patterns for non-exNA-
containing crRNAs

| Name | Sequence |
|---|---|
| crRNA 1 | mN#mN#mN#mNmNmNmNmNmNmNrNrNrNrNrNmNmN |
| | mNrNmNmGrUrUrUmUmAmGmAmGmCmUmAmU#mG# |
| | mCmU(SEQ ID NO: 43) |
| crRNA 2 | rNrNrNrNrNrNmNmNmNmNrNrNrNrNrNrNrNrNrNmNm |
| | GrUrUrUrUrAmGmAmGmCmUmAmU#mG#mCmU(SEQ |
| | ID NO: 44) |
| crRNA 3 | rN#rN#rN#rNrNrNmNmNmNmNrNrNrNrNrNrNrNrNrNm |
| | NmGrUrUrUrUrAmGmAmGmCmUmAmU#mG#mCmU |
| | (SEQ ID NO: 45) |
| crRNA 4 | mN#mN#mN#mNmNmNmNmNmNmNrNrNrNrNrNrNrNr |
| | NrNmNmGrUrUrUrUrAmGmAmGmCmUmAmU#mG#mC |
| | #mU(SEQ ID NO: 46) |
| crRNA 5 | rN#rN#rN#rNrNrNmNmNmNmNrNrNrNrNrNrNmNmNrN |
| | mNmGrUrUrUrUrAmGmAmGmCmUmAmU#mG#mC#mU |
| | (SEQ ID NO: 47) |
| crRNA 6 | rN#rN#rN#rNrNrNmNmNmNmNrNrNrNrNrNrNrNrNrNm |
| | NmGrUrUrUmUmAmGmAmGmCmUmAmU#mG#mC#mU |
| | (SEQ ID NO: 48) |
| crRNA 7 | mN#mN#mN#mNmNmNmNmNmNmNrNrNrNrNrNrNmN |
| | mNrNmNmGrUrUrUrUrAmGmAmGmCmUmAmU#mG#m |
| | C#mU(SEQ ID NO: 49) |
| crRNA 8 | mN#mN#mN#mNmNmNmNmNmNmNrNrNrNrNrNrNrNr |
| | NrNmNmGrUrUrUmUmAmGmAmGmCmUmAmU#mG#m |
| | C#mU(SEQ ID NO: 50) |
| crRNA 9 | mN#mN#mN#mNmNmNmNmNmNmNrNrNrNrNrN#rN#r |
| | NrNrN#mNmGrU#rU#rU#mUmAmGmAmGmCmUmAmU |
| | #mG#mC#mU(SEQ ID NO: 51) |
| crRNA 10 | mN#mN#mN#mNmNmNmNmNmNmNfNfNfNfNrN#rN#f |
| | NfNrN#mNmGrU#rU#rU#mUmAmGmAmGmCmUmAmU |
| | #mG#mC#mU(SEQ ID NO: 52) |
| crRNA 11 | mN#mN#mN#mNmNmNmNmNmNmNfNfNfNfNrN#rN#r |
| | N#rN#rN#mNmGrU#rU#rU#mUmAmGmAmGmCmUmAm |
| | U#mG#mC#mU(SEQ ID NO: 53) |
| crRNA 17 | mN#mN#mN#mNmNmNmNmNmNmNfNfNfNfNrN#rN#f |
| | NfNrN#mNmGrU#rU#rU#mUrA#mGmAmGmCmUmAmU |
| | #mG#mC#mU(SEQ ID NO: 54) |
| crRNA 18 | mN#mN#mN#mNmNmNmNmNmNmNfNfNfNfNrN#rN#f |
| | NfNrN#mNmGrU#rU#rU#rU#mAmGmAmGmCmUmAmU |
| | #mG#mC#mU(SEQ ID NO: 55) |
| crRNA 19 | mN#mN#mN#mNmNmNmNmNmNmNfNfNfNfNrN#rN#f |
| | NfNrN#mNmGrU#rU#rU#rU#rA#mGmAmGmCmUmAmU |
| | #mG#mC#mU(SEQ ID NO: 56) |
| crRNA 20 | mN#mN#mN#mNmNmNmNmNmNmNfNfNfNfNrN#rN#f |
| | NfNrN#mNmGrU#rU#rU#fUfAmGmAmGmCmUmAmU# |
| | mG#mC#mU(SEQ ID NO: 57) |
| crRNA 21 | mN#mN#mN#mNmNmNmNmNmNmNfNfNfNfNfNfNfNf |
| | NfNmNmGfUfUfUfUfAmGmAmGmCmUmAmU#mG#mC |
| | #mU(SEQ ID NO: 58) |
| crRNA 22 | mN#mN#mN#mNmNmNmNmNmNmNfNfNfNfNrN#rN#f |
| | NfNrN#mNmGfUrU#fUfUfAmGmAmGmCmUmAmU#mG |
| | #mC#mU(SEQ ID NO: 59) |
| crRNA 23 | mN#mN#mN#mNmNmNmNmNmNmNfNfNfNfNmNrN#f |
| | NfNrN#mNmGrU#rU#rU#fUfAmGmAmGmCmUmAmU# |
| | mG#mC#mU(SEQ ID NO: 60) |
| crRNA 24 | mN#mN#mN#mNmNmNmNmNmNmNfNfNfNfNrN#fNfNf |
| | NrN#mNmGrU#rU#rU#fUfAmGmAmGmCmUmAmU#mG |
| | #mC#mU(SEQ ID NO: 61) |
| crRNA 25 | mN#mN#mN#mNmNmNmNmNmNmNfNfNfNfNrN#rN#f |
| | NfNfNmNmGrU#rU#rU#fUfAmGmAmGmCmUmAmU#m |
| | G#mC#mU(SEQ ID NO: 62) |
| crRNA 26 | mN#mN#mN#mNmNmNmNmNmNmNfNfNfNfNrN#rN#f |
| | NfNrN#mNmGfUrU#rU#fUfAmGmAmGmCmUmAmU#m |
| | G#mC#mU(SEQ ID NO: 63) |
| crRNA 27 | mN#mN#mN#mNmNmNmNmNmNmNfNfNfNfNrN#rN#f |
| | NfNrN#NAmGrU#fUrU#fUfAmGmAmGmCmUmAmU#m |
| | G#mC#mU(SEQ ID NO: 64) |
| crRNA 28 | mN#mN#mN#mNmNmNmNmNmNmNfNfNfNfNrN#rN#f |
| | NfNrN#mNmGrU#rU#rUfUfAmGmAmGmCmUmAmU#m |
| | G#mC#Mu(SEQ ID NO: 65) |
| crRNA 29 | mN#mN#mN#mNmNmNmNmNmNmNfNfNfNfNrN#fNfNf |
| | NfNmNmGfUfUfUfUfAmGmAmGmCmUmAmU#mG#mC |
| | #mU(SEQ ID NO: 66) |
| crRNA 30 | mN#mN#mN#rNrNrNmNmNmNmNrNrNrNrNrNrNrNrNrN |
| | mNmGrUrUrUrUrAmGmAmGmCmUmAmU#mG#mC#mU |
| | (SEQ ID NO: 67) |
| crRNA 31 | mN#mN#mN#rNrNrNmNmNmNmNmNrNrNrNrNrNrNrNr |
| | NmNmGrUrUrUrUrAmGmAmGmCmUmAmU#mG#mC#m |
| | U(SEQ ID NO: 68) |
| crRNA 32 | mN#mN#mN#rNrNrNmNmNmNmNmNrNmNmNrNrNrNr |
| | NrNmNmGrUrUrUrUrAmGmAmGmCmUmAmU#mG#mC |
| | #mU(SEQ ID NO: 69) |
| crRNA 33 | mN#mN#mN#rN#rN#rN#mNmNmNmNrN#rN#rN#rN#rN#r |
| | N#rN#rN#rN#mNmGrU#rU#rU#rU#rA#mGmAmGmCmU |
| | mAmU#mG#mC#mU(SEQ ID NO: 70) |
| crRNA 34 | mN#mN#mN#rN#rN#rN#mNmNmNmNrN#rN#rN#rN#rN#r |
| | N#rN#rN#rN#mNmGrUrUrUrUrAmGmAmGmCmUmAmU |
| | #mG#mC#mU(SEQ ID NO: 71) |
| crRNA 35 | mN#mN#mN#rNrNrNmNmNmNmNrNrNrNrNrNrNrNrNrN |
| | mNmGrUrUrUmUmAmGmAmGmCmUmAmU#mG#mC# |
| | mU(SEQ ID NO: 72) |
| crRNA 36 | mN#mN#mN#rN#rN#rN#mNmNmNmNrN#rN#rN#rN#rN#r |
| | N#rN#rN#rN#mNmGrUrUrUmUmAmGmAmGmCmUmAm |
| | U#mG#mC#mU(SEQ ID NO: 73) |
| crRNA 37 | mN#mN#mN#rN#rN#rN#mNmNmNmNfNfNfNfNrN#rN#f |
| | NfNrN#mNmGrU#rU#rU#mUmAmGmAmGmCmUmAmU |
| | #mG#mC#mU(SEQ ID NO: 74) |
| crRNA 38 | mN#mN#mN#dN#dN#dN#mNmNmNmNfNfNfNfNrN#rN# |
| | fNfNrN#mNmGrU#rU#rU#mUmAmGmAmGmCmUmAmU |
| | #mG#mC#mU(SEQ ID NO: 75) |
| crRNA 39 | mN#mN#mN#rN#rN#rN#mNmNmNmNfNfNfNfNfNfNfNf |
| | NfNmNmGfUfUfUfUfAmGmAmGmCmUmAmU#mG#mC |
| | #mU(SEQ ID NO: 76) |
| crRNA 40 | mN#mN#mN#dN#dN#dN#mNmNmNmNfNfNfNfNfNfNfN |
| | fNfNmNmGfUfUfUfUfAmGmAmGmCmUmAmU#mG#mC |
| | #mU(SEQ ID NO: 77) |
| crRNA 41 | mN#mN#mN#mNmNmNmNmNmNmNfNfNfNfNdN#dN#f |
| | NfNdN#mNmGrU#rU#rU#fUfAmGmAmGmCmUmAmU# |
| | mG#mC#mU(SEQ ID NO: 78) |
| crRNA 42 | mN#mN#mN#mNmNmNmNmNmNmNfNfNfNfNrN#rN#f |
| | NfNrN#mNmGdU#dU#dU#fUfAmGmAmGmCmUmAmU# |
| | mG#mC#mU(SEQ ID NO: 79) |
| crRNA 43 | mN#mN#mN#mNmNmNmNmNmNmNfNrN#fNfNrN#rN#f |
| | NfNrN#mNmGrU#rU#rU#fUfAmGmAmGmCmUmAmU# |
| | mG#mC#mU(SEQ ID NO: 80) |
| crRNA 44 | mN#mN#mN#mNmNmNmNmNmNmNfNdN#fNfNrN#rN# |
| | fNfNrN#mNmGrU#rU#rU#fUfAmGmAmGmCmUmAmU# |
| | mG#mC#mU(SEQ ID NO: 81) |
| crRNA 45 | mN#mN#mN#fNfNfNmNmNmNmNfNfNfNfNfNfNfNfNfN |
| | mNmGfUfUfUfUfAmGmAmGmCmUmAmU#mG#mC#mU |
| | (SEQ ID NO: 82) |
| crRNA 46 | mN#mN#mN#mNmNmNmNmNmNmNfNfNfNfNmNrN#f |
| | NfNrN#mNmGrU#rUrU#fUfAmGmAmGmCmUmAmU#m |
| | G#mC#mU(SEQ ID NO: 83) |
| crRNA 47 | mN#mN#mN#mNmNmNmNmNmNmNfNfNfNfNrN#mNf |
| | NfNrN#mNmGrU#rUrU#fUfAmGmAmGmCmUmAmU#m |
| | G#mC#mU(SEQ ID NO: 84) |
| crRNA 48 | mN#mN#mN#mNmNmNmNmNmNmNfNfNfNfNrN#mNf |
| | NfNmNmNmGrU#rUrU#fUfAmGmAmGmCmUmAmU#m |
| | G#mC#mU(SEQ ID NO: 85) |
| crRNA 49 | mN#mN#mN#mNmNmNmNmNmNmNfNfNfNfNrN#rN#f |
| | NfNrN#mNmGmUrU#rU#fUfAmGmAmGmCmUmAmU#m |
| | G#mC#mU(SEQ ID NO: 86) |
| crRNA 50 | mN#mN#mN#mNmNmNmNmNmNmNfNfNfNfNrN#rN#f |
| | NfNrN#mNmGrU#mUrU#fUfAmGmAmGmCmUmAmU#m |
| | G#mC#mU(SEQ ID NO: 87) |
| crRNA 51 | mN#mN#mN#mNmNmNmNmNmNmNfNfNfNfNrN#rN#f |
| | NfNrN#mNmGrU#rU#mUfUfAmGmAmGmCmUmAmU#m |
| | G#mC#mU(SEQ ID NO: 88) |
| crRNA 52 | mN#mN#mN#dN#dN#dN#mNmNmNmNfNfNfNfNfN#fN# |
| | fNfNfN#mNmGfU#fU#fU#fU#fA#mGmAmGmCmUmAm |
| | U#mG#mC#mU(SEQ ID NO: 89) |
| crRNA 53 | mN#mN#mN#dN#dN#dN#mN#mNmNmNfNfNfNfNfNfNf |
| | NfNfNmNmGfUfUfUfUfAmGmAmGmCmUmAmU#mG# |
| | mC#mU(SEQ ID NO: 90) |
| crRNA 54 | mN#mN#mN#dN#dN#dN#mNmN#mNmNfNfNfNfNfNfNf |
| | NfNfNmNmGfUfUfUfUfAmGmAmGmCmUmAmU#mG# |
| | mC#mU(SEQ ID NO: 91) |
| crRNA 55 | mN#mN#mN#dN#dN#dN#mNmNmN#mNfNfNfNfNfNfNf |
| | NfNfNmNmGfUfUfUfUfAmGmAmGmCmUmAmU#mG# |
| | mC#mU(SEQ ID NO: 92) |
| crRNA 56 | mN#mN#mN#dN#dN#dN#mNmNmNmN#fNfNfNfNfNfNf |
| | NfNfNmNmGfUfUfUfUfAmGmAmGmCmUmAmU#mG# |
| | mC#mU(SEQ ID NO: 93) |
| crRNA 57 | mN#mN#mN#dN#dN#dN#mNmNmNmNfN#fNfNfNfNfNf |
| | NfNfNmNmGfUfUfUfUfAmGmAmGmCmUmAmU#mG# |
| | mC#mU(SEQ ID NO: 94) |
| crRNA 58 | mN#mN#mN#dN#dN#dN#mNmNmNmNfNfN#fNfNfNfNf |
| | NfNfNmNmGfUfUfUfUfAmGmAmGmCmUmAmU#mG# |
| | mC#mU(SEQ ID NO: 95) |
| crRNA 59 | mN#mN#mN#dN#dN#dN#mNmNmNmNfNfNfN#fNfNfNf |
| | NfNfNmNmGfUfUfUfUfAmGmAmGmCmUmAmU#mG# |
| | mC#mU(SEQ ID NO: 96) |
| crRNA 60 | mN#mN#mN#dN#dN#dN#mNmNmNmNfNfNfNfN#fNfNf |
| | NfNfNmNmGfUfUfUfUfAmGmAmGmCmUmAmU#mG# |
| | mC#mU(SEQ ID NO: 97) |
| crRNA 61 | mN#mN#mN#dN#dN#dN#mNmNmNmNfNfNfNfNfNfNfN |
| | #fNfNmNmGfUfUfUfUfAmGmAmGmCmUmAmU#mG#m |
| | C#mU(SEQ ID NO: 98) |
| crRNA 62 | mN#mN#mN#dN#dN#dN#mNmNmNmNfNfNfNfNfNfNfN |
| | fN#fNmNmGfUfUfUfUfAmGmAmGmCmUmAmU#mG#m |
| | C#mU(SEQ ID NO: 99) |
| crRNA 63 | mN#mN#mN#dN#dN#dN#mNmNmNmNfNfNfNfNfNfNfN |
| | fNfNmN#mGfUfUfUfUfAmGmAmGmCmUmAmU#mG#m |
| | C#mU(SEQ ID NO: 100) |
| crRNA 64 | mN#mN#mN#dN#dN#dN#mNmNmNmNfNfNfNfNfNfNfN |
| | fNfNmNmG#fUfUfUfUfAmGmAmGmCmUmAmU#mG#m |
| | C#mU(SEQ ID NO: 101) |
| crRNA 65 | mN#mN#mN#dN#dN#dN#mNmNmNmNfNfNfNfNfNfNfN |
| | fNfNmNmGfUfUfUfUfAmG#mAmGmCmUmAmU#mG#m |
| | C#mU(SEQ ID NO: 102) |
| crRNA 66 | mN#mN#mN#dN#dN#dN#mNmNmNmNfNfNfNfNfNfNfN |
| | fNfNmNmGfUfUfUfUfAmGmA#mGmCmUmAmU#mG#m |
| | C#mU(SEQ ID NO: 103) |
| crRNA 67 | mN#mN#mN#dN#dN#dN#mNmNmNmNfNfNfNfNfNfNfN |
| | fNfNmNmGfUfUfUfUfAmGmAmG#mCmUmAmU#mG#m |
| | C#mU(SEQ ID NO: 104) |
| crRNA 68 | mN#mN#mN#dN#dN#dN#mNmNmNmNfNfNfNfNfNfNfN |
| | fNfNmNmGfUfUfUfUfAmGmAmGmC#mUmAmU#mG#m |
| | C#mU(SEQ ID NO: 105) |
| crRNA 69 | mN#mN#mN#dN#dN#dN#mNmNmNmNfNfNfNfNfNfNfN |
| | fNfNmNmGfUfUfUfUfAmGmAmGmCmU#mAmU#mG#m |
| | C#mU(SEQ ID NO: 106) |
| crRNA 70 | mN#mN#mN#dN#dN#dN#mNmNmNmNfNfNfNfNfNfNfN |
| | fNfNmNmGfUfUfUfUfAmGmAmGmCmUmA#mU#mG#m |
| | C#mU(SEQ ID NO: 107) |
| crRNA 71 | mN#mN#mN#dN#dN#dN#mN#mN#mN#mNfNfNfNfNfN#f |
| | N#fNfNfN#mNmGfU#fU#fU#fU#fA#mGmAmGmCmUmA |
| | mU#mG#mC#mU(SEQ ID NO: 108) |
| crRNA 72 | mN#mN#mN#dN#dN#dN#mNmNmNmN#fN#fN#fNfNfN#f |
| | N#fNfNfN#mNmGfU#fU#fU#fU#fA#mGmAmGmCmUmA |
| | mU#mG#mC#mU(SEQ ID NO: 109) |
| crRNA 73 | mN#mN#mN#dN#dN#dN#mNmNmNmNfNfNfN#fN#fN#f |
| | N#fNfNfN#mNmGfU#fU#fU#fU#fA#mGmAmGmCmUmA |
| | mU#mG#mC#mU(SEQ ID NO: 110) |
| crRNA 74 | mN#mN#mN#dN#dN#dN#mNmNmNmNfNfNfNfNfN#fN# |
| | fN#fN#fN#mNmGfU#fU#fU#fU#fA#mGmAmGmCmUmA |
| | mU#mG#mC#mU(SEQ ID NO: 111) |
| crRNA 75 | mN#mN#mN#dN#dN#dN#mNmNmNmNfNfNfNfNfN#fN# |
| | fNfNfN#mN#mGfU#fU#fU#fU#fA#mGmAmGmCmUmAm |
| | U#mG#mC#mU(SEQ ID NO: 112) |
| crRNA 76 | mN#mN#mN#dN#dN#dN#mN#mN#mN#mNfNfNfNfNfNf |
| | NfNfNfNmNmGfUfUfUfUfAmGmAmGmCmUmAmU#mG |
| | #mC#mU(SEQ ID NO: 113) |
| crRNA 77 | mN#mN#mN#dN#dN#dN#mN#mN#mN#mN#fN#fN#fNfNf |
| | NfNfNfNfNmNmGfUfUfUfUfAmGmAmGmCmUmAmU# |
| | mG#mC#mU(SEQ ID NO: 114) |
| crRNA 78 | mN#mN#mN#dN#dN#dN#mN#mN#mN#mN#fN#fN#fN#f |
| | N#fN#fN#fNfNfNmNmGfUfUfUfUfAmGmAmGmCmUmA |
| | mU#mG#mC#mU(SEQ ID NO: 115) |
| crRNA 79 | mN#mN#mN#dN#dN#dN#mN#mN#mN#mN#fN#fN#fN#f |
| | N#fN#fN#fN#fN#fN#mNmGfUfUfUfUfAmGmAmGmCmU |
| | mAmU#mG#mC#mU(SEQ ID NO: 116) |
| crRNA 80 | mN#mN#mN#dN#dN#dN#mN#mN#mN#mN#fN#fN#fN#f |
| | N#fN#fN#fN#fN#fN#mN#mG#fU#fU#fU#fU#fA#mGmAm |
| | GmCmUmAmU#mG#mC#mU(SEQ ID NO: 117) |
| crRNA 81 | mN#mN#mN#dN#dN#dN#mNmNmNmNfNfNfNfNfN#fN# |
| | fNfNfN#mNmGfU#fU#fU#fU#fA#mGmAmGmC#mU#mA |
| | #mU#mG#mC#mU(SEQ ID NO: 118) |
| crRNA 82 | mN#mN#mN#dN#dN#dN#mNmNmNmNfNfNfNfNfN#fN# |
| | fNfNfN#mNmGfU#fU#fU#fU#fA#mG#mA#mG#mCmUm |
| | AmU#mG#mC#mU(SEQ ID NO: 119) |
| crRNA 83 | mN#mN#mN#dN#dN#dN#mNmNmNmNfNfNfNfNfN#fN# |
| | fNfNfN#mNmG#fU#fU#fU#fU#fA#mGmAmGmCmUmAm |
| | U#mG#mC#mU(SEQ ID NO: 120) |
| crRNA 84 | mN#mN#mN#dN#dN#dN#mNmNmNmNfNfNfNfNfNfNfN |
| | fNfNmNmGfUfUfUfUfAmGmAmGmC#mU#mA#mU#mG |
| | #mC#mU(SEQ ID NO: 121) |
| crRNA 85 | mN#mN#mN#dN#dN#dN#mNmNmNmNfNfNfNfNfNfNfN |
| | fNfNmNmGfUfUfUfUfA#mG#mA#mG#mC#mU#mA#mU |
| | #mG#mC#mU(SEQ ID NO: 122) |
| crRNA 86 | mN#mN#mN#dN#dN#dN#mNmNmNmNfNfNfNfNfNfNfN |
| | fNfNmNmGfU#fU#fU#fU#fA#mG#mA#mG#mC#mU#mA |
| | #mU#mG#mC#mU(SEQ ID NO: 123) |
| crRNA 87 | mN#mN#mN#dN#dN#dN#mN#mN#mNmNfNfNfNfNfNfN |
| | fNfNfNmNmGfUfUfUfUfAmGmAmGmCmU#mA#mU#m |
| | G#mC#mU(SEQ ID NO: 124) |
| crRNA 88 | mN#mN#mN#dN#dN#dN#mN#mN#mN#mN#fNfNfNfNfNf |
| | NfNfNfNmNmGfUfUfUfUfAmGmAmG#mC#mU#mA#mU |
| | #mG#mC#mU(SEQ ID NO: 125) |
| crRNA 89 | mN#mN#mN#dN#dN#dN#mN#mN#mN#mN#fN#fN#fNfNf |
| | NfNfNfNfNmNmGfUfUfUfUfAmG#mA#mG#mC#mU#mA |
| | #mU#mG#mC#mU(SEQ ID NO: 126) |
| crRNA 90 | mN#mN#mN#dN#dN#dN#mN#mN#mN#mN#fN#fN#fN#f |
| | N#fNfNfNfNfNmNmGfUfUfUfU#fA#mG#mA#mG#mC#m |
| | U#mA#mU#mG#mC#mU(SEQ ID NO: 127) |
| crRNA 91 | mN#mN#mN#dN#dN#dN#mN#mN#mN#mN#fN#fN#fN#f |
| | N#fN#fN#fNfNfNmNmGfUfU#fU#fU#fA#mG#mA#mG#m |
| | C#mU#mA#mU#mG#mC#mU(SEQ ID NO: 128) |
| crRNA 92 | mN#mN#mN#dN#dN#dN#mN#mN#mN#mN#fN#fN#fN#f |
| | N#fN#fN#fN#fN#fN#mNmGfU#fU#fU#fU#fA#mG#mA#m |
| | G#mC#mU#mA#mU#mG#mC#mU(SEQ ID NO: 129) |
| crRNA 93 | mN#mN#mN#dN#dN#dN#mN#mN#mN#mN#fN#fN#fN#f |
| | N#fN#fN#fN#fN#fN#mN#mG#fU#fU#fU#fU#fA#mG#mA# |
| | mG#mC#mU#mA#mU#mG#mC#mU(SEQ ID NO: 130) |
| crRNA 94 | mN#mN#mN#mN#mN#mN#mNmNmNmNfNfNfNfNrN#r |
| | N#fNfNrN#mNmGrU#rU#rU#fUfAmGmAmGmCmUmAm |
| | U#mG#mC#mU(SEQ ID NO: 131) |
| crRNA 95 | mN#mN#mN#mN#mN#mN#mN#mNmNmNfNfNfNfNrN#r |
| | N#fNfNrN#mNmGrU#rU#rU#fUfAmGmAmGmCmUmAm |
| | U#mG#mC#mU(SEQ ID NO: 132) |
| crRNA 96 | mN#mN#mN#mN#mN#mN#mNmNmNmNfNfNfNfNrN#r |
| | N#fNfNrN#mNmGrU#rU#rU#fUfAmGmAmGmC#mUmA |
| | mU#mG#mC#mU(SEQ ID NO: 133) |
| crRNA 97 | mN#mN#mN#mN#mN#mN#mNmNmNmNfNfNfNfNrN#r |
| | N#fNfNrN#mNmGrU#rU#rU#fUfAmGmAmGmCmU#mA |
| | mU#mG#mC#mU(SEQ ID NO: 134) |
| crRNA 98 | mN#mN#mN#mN#mN#mN#mNmNmNmNfNfNfNfNrN#r |
| | N#fNfNrN#mGNmGrU#rU#rU#fUfAmGmAmGmCmUmA |
| | #mU#mG#mC#mU(SEQ ID NO: 135) |
| crRNA 99 | mN#mN#mN#mN#mN#mN#mNmNmNmNfNfNfNfNrN#r |
| | N#fNfNrN#mNmGrU#rU#rU#fU#fA#mGmAmGmCmUmA |
| | mU#mG#mC#mU(SEQ ID NO: 136) |
| crRNA 100 | mN#mN#mN#mN#mN#mN#mNmNmNmNfNfNfNfNrN#r |
| | N#fNfNrN#mNmGrU#rU#rU#fU#fA#mGmAmGmC#mU# |
| | mA#mU#mG#mC#mU(SEQ ID NO: 137) |
| crRNA 101 | mN#mN#mN#mN#mN#mN#mNmNmNmNfNfNfNfNrN#r |
| | N#fNfNrN#mNmGrU#rU#rU#fU#fA#mG#mA#mG#mCmU |
| | mAmU#mG#mC#mU(SEQ ID NO: 138) |
| crRNA 102 | mN#mN#mN#mN#mN#mN#mNmNmNmNfNfNfNfNrN#r |
| | N#fNfNrN#mNmG#rU#rU#rU#fU#fA#mGmAmGmCmUm |
| | AmU#mG#mC#mU(SEQ ID NO: 139) |
| crRNA 103 | mN#mN#mN#mN#mN#mN#mNmNmNmNfNfNfNfNrN#r |
| | N#fNfNrN#mNmGrU#rU#rU#fUfAmGmAmGmC#mU#mA |
| | #mU#mG#mC#mU(SEQ ID NO: 140) |
| crRNA 104 | mN#mN#mN#mN#mN#mN#mNmNmNmNfNfNfNfNrN#r |
| | N#fNfNrN#mNmGrU#rU#rU#fUfA#mG#mA#mG#mC#mU |
| | #mA#mU#mG#mC#mU(SEQ ID NO: 141) |
| crRNA 105 | mN#mN#mN#rN#rN#rN#mN#mNmNmNfNfNfNfNfNfNfN |
| | fNfNmNmGfUfUfUfUfAmGmAmGmCmUmAmU#mG#mC |
| | #mU(SEQ ID NO: 142) |
| crRNA 106 | mN#mN#mN#rN#rN#rN#mNmNmNmNfNfNfNfNfNfNfNf |
| | NfNmNmGfUfUfUfUfAmGmAmGmCmUmA#mU#mG#m |
| | C#mU(SEQ ID NO: 143) |
| crRNA 107 | mN#mN#mN#rN#rN#rN#mNmNmNmNfNfNfNfNfN#fN#f |
| | N#fN#fN#mNmGfU#fU#fU#fU#fA#mGmAmGmCmUmA |
| | mU#mG#mC#mU(SEQ ID NO: 144) |
| crRNA 108 | mN#mN#mN#rN#rN#rN#mNmNmNmNfNfNfNfNfN#fN#f |
| | NfNfN#mNmGfU#fU#fU#fU#fA#mGmAmGmC#mU#mA# |
| | mU#mG#mC#mU(SEQ ID NO: 145) |
| crRNA 109 | mN#mN#mN#rN#rN#rN#mNmNmNmNfNfNfNfNfN#fN#f |
| | NfNfN#mNmGfU#fU#fU#fU#fA#mG#mA#mG#mCmUmA |
| | mU#mG#mC#mU(SEQ ID NO: 146) |
| crRNA 110 | mN#mN#mN#rN#rN#rN#mNmNmNmNfNfNfNfNfN#fN#f |
| | NfNfN#mNmG#fU#fU#fU#fU#fA#mGmAmGmCmUmAm |
| | U#mG#mC#mU(SEQ ID NO: 147) |
| crRNA 111 | mN#mN#mN#rN#rN#rN#mNmNmNmNfNfNfNfNfNfNfNf |
| | NfNmNmGfUfUfUfUfAmGmAmGmC#mU#mAmU#mG#m |
| | C#mU(SEQ ID NO: 148) |
| crRNA 112 | mN#mN#mN#rN#rN#rN#mNmNmNmNfNfNfNfNfNfNfNf |
| | NfNmNmGfUfUfUfUfA#mG#mA#mG#mC#mU#mAmU#m |
| | G#mC#mU(SEQ ID NO: 149) |
| crRNA 113 | mN#mN#mN#dN#dN#dN#mNmNmNmNfNfNfNfNdN#dN |
| | #fNfNdN#mNmGdU#dU#dU#fUfAmGmAmGmCmUmAm |
| | U#mG#mC#mU(SEQ ID NO: 150) |
| crRNA 114 | mN#mN#mN#mNmNmNmNmNmNmNfNfNfNfNrN#rN#f |
| | NfNaNmNmGaUaUaUfUfAmGmAmGmCmUmAmU#mG# |
| | mC#mU(SEQ ID NO: 151) |
| crRNA 115 | mN#mN#mN#mNmNmNmNmNmNmNfNfNfNfNrN#rN#f |
| | NfNaNmNmGrU#rU#rU#fUfAmGmAmGmCmUmAmU#m |
| | G#mC#mU(SEQ ID NO: 152) |
| crRNA 116 | mN#mN#mN#mNmNmNmNmNmNmNfNfNfNfNrN#rN#f |
| | NfNrN#mNmGaUrU#rU#fUfAmGmAmGmCmUmAmU#m |
| | G#mC#mU(SEQ ID NO: 153) |
| crRNA 117 | mN#mN#mN#mNmNmNmNmNmNmNfNfNfNfNrN#rN#f |
| | NfNrN#mNmGrU#aUrU#fUfAmGmAmGmCmUmAmU#m |
| | G#mC#mU(SEQ ID NO: 154) |
| crRNA 118 | mN#mN#mN#mNmNmNmNmNmNmNfNfNfNfNrN#rN#f |
| | NfNrN#mNmGrU#rU#aUfUfAmGmAmGmCmUmAmU#m |
| | G#mC#mU(SEQ ID NO: 155) |
| crRNA 119 | mN#mN#mN#mNmNmNmNmNmNmNfNfNfNfNrN#rN#f |
| | NfNsN#mNmGsU#sU#sU#fUfAmGmAmGmCmUmAmU# |
| | mG#mC#mU(SEQ ID NO: 156) |
| crRNA 120 | mN#mN#mN#mNmNmNmNmNmNmNfNfNfNfNrN#rN#f |
| | NfNsNmNmGsUsUsUfUfAmGmAmGmCmUmAmU#mG# |
| | mC#mU(SEQ ID NO: 157) |
| crRNA 121 | mN#mN#mN#mNmNmNmNmNmNmNfNfNfNfNrN#rN#f |
| | NfNsNmNmGrU#rU#rU#fUfAmGmAmGmCmUmAmU#m |
| | G#mC#mU(SEQ ID NO: 158) |
| crRNA 122 | mN#mN#mN#mNmNmNmNmNmNmNfNfNfNfNrN#rN#f |
| | NfNrN#mNmGsUrU#rU#fUfAmGmAmGmCmUmAmU#m |
| | G#mC#mU(SEQ ID NO: 159) |
| crRNA 123 | mN#mN#mN#mNmNmNmNmNmNmNfNfNfNfNrN#rN#f |
| | NfNrN#mNmGrU#sUrU#fUfAmGmAmGmCmUmAmU#m |
| | G#mC#mU(SEQ ID NO: 160) |
| crRNA 124 | mN#mN#mN#mNmNmNmNmNmNmNfNfNfNfNrN#rN#f |
| | NfNrN#mNmGrU#rU#sUfUfAmGmAmGmCmUmAmU#m |
| | G#mC#mU(SEQ ID NO: 161) |
| crRNA 125 | mN#mN#mN#mNmNmNmNmNmNmNfNfNfNfNrN#rN#f |
| | NfNrN#mNmGsUrU#sUfUfAmGmAmGmCmUmAmU#mG |
| | #mC#mU(SEQ ID NO: 162) |
| crRNA 126 | mN#mN#mN#mNmNmNmNmNmNmNfNfNfNfNrN#rN#f |
| | NfNrN#mNmGsUsUrU#fUfAmGmAmGmCmUmAmU#mG |
| | #mC#mU(SEQ ID NO: 163) |
| crRNA 127 | mN#mN#mN#mNmNmNmNmNmNmNfNfNfNfNrN#rN#f |
| | NfNrN#mNmGrU#sUsUfUfAmGmAmGmCmUmAmU#mG |
| | #mC#mU(SEQ ID NO: 164) |
| crRNA 128 | mN#mN#mN#mNmNmNmNmNmNmNfNfNfNfNrN#rN#f |
| | NfNrN#mNmGaUaUaUfUfAmGmAmGmCmUmAmU#mG |
| | #mC#mU(SEQ ID NO: 165) |
| crRNA 129 | mN#mN#mN#mNmNmNmNmNmNmNfNfNfNfNrN#rN#f |
| | NfNrN#mNmGsU#sU#sU#fUfAmGmAmGmCmUmAmU# |
| | mG#mC#mU (SEQ ID NO: 166) |
| crRNA 130 | mN#mN#mN#mNmNmNmNmNmNmNfNfNfNfNrN#rN#f |
| | NfNrN#mNmGsUsUsUfUfAmGmAmGmCmUmAmU#mG# |
| | mC#mU (SEQ ID NO: 167) |
| crRNA 131 | mN#mN#mN#mNmNmNmNmNmNmNfNfNfNfNrN#aNfN |
| | fNrN#mNmGrU#rU#rU#fUfAmGmAmGmCmUmAmU#m |
| | G#mC#mU (SEQ ID NO: 168) |
| crRNA 132 | mN#mN#mN#mNmNmNmNmNmNmNfNfNfNfNrN#rN#f |
| | NfNaNmNmGrU#rU#rU#fUfAmGmAmGmCmUmAmU#m |
| | G#mC#mU (SEQ ID NO: 169) |
| crRNA 133 | mN#mN#mN#mNmNmNmNmNmNmNfNfNfNfNrN#aN#f |
| | NfNaN#mNmGrU#rU#rU#fUfAmGmAmGmCmUmAmU# |
| | mG#mC#mU (SEQ ID NO: 170) |
| crRNA 134 | mN#mN#mN#mNmNmNmNmNmNmNfNfNfNfNrN#rN#f |
| | NfNrN#mNmGaUaUaUfUfAmGmAmGmCmUmAmU#mG |
| | #mC#mU(SEQ ID NO: 165) |

KEY:
rN = RNA, mN = 2′-O-methyl RNA, fN = 2′-fluoro RNA, aN = 2′-NH₂ (2′-amino RNA), sN = 4′-thio RNA, dN = 2′-deoxy RNA, N = A, U, G, or C N#N = phosphorothioate linkage

TABLE 2
Exemplary chemical modification patterns
for non-exNA-containing tracrRNAs
KEY: rN = RNA, mN = 2′-O-methyl RNA, fN =
2′-fluoro RNA, sN = 4′-thio RNA, dN = 2′-
deoxy RNA, N = A, U, G, or C N#N =
phosphorothioate linkage

| Name | Sequence |
|---|---|
| tracrRNA | mA#mG#mC#mAmUmAmGrCrArArGrUrUmAr |
| 1 | ArArArUrArArGmGrCrUmArGrUrCmCrGr |
| | UrUrArUrCrAmAmCmUmUmGmAmAmAmAmAm |
| | GmUrGrGrCrAmCmCmGrArGrUrCrGmGmUm |
| | GmC#mU#mU#Mu |
| | (SEQ ID NO: 171) |
| tracrRNA | mA#mG#mC#mAmUmAmGmCmAmAmGrUrUmAr |
| 2 | AmAmArUmAmAmGmGrCrUmArGrUrCmCrGr |
| | UrUmAmUmCmAmAmCmUmUmGmAmAmAmAmAm |
| | mGmUGmGmCmAmCmCmGmAmGmUmCmGmGmUm |
| | GmC#mU#mU#mU |
| | (SEQ ID NO: 15) |
| tracrRNA | mA#mG#mC#mAmUmAmGmCmAmAmGrU#rU#m |
| 3 | ArA#mAmArU#mAmAmGmGrC#rU#mArG#rU |
| | #rC#mCrG#rU#rU#mAmUmCmAmAmCmUmUm |
| | GmAmAmAmAmAmGmUmGmGmCmAmCmCmGmAm |
| | GmUmCmGmGmUmGmC#mU#mU#mU |
| | (SEQ ID NO: 172) |
| tracrRNA | mA#mG#mC#mAmUmAmGmCmAmAmGrUrUmAm |
| 4 | AmAmArUmAmAmGmGrCrUmArGrUrCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 173) |
| tracrRNA | mA#mG#mC#mAmUmAmGmCmAmAmGrUrUmAm |
| 5 | AmAmArUmAmAmGmGrCrUmArGrUrCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 173) |
| tracrRNA | mA#mG#mC#mAmUmAmGmCmAmAmGrUrUmAr |
| 6 | AmAmAfUmAmAmGmGfCfUmArGfUfCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#Mu |
| | (SEQ ID NO: 174) |
| tracrRNA | mA#mG#mC#mAmUmAmGmCmAmAmGrUfUmAr |
| 7 | AmAmAfUmAmAmGmGfCfUmAfGfUfCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 175) |
| tracrRNA | mA#mG#mC#mAmUmAmGmCmAmAmGfUfUmAf |
| 8 | AmAmAfUmAmAmGmGfCfUmAfGfUfCmCfGf |
| | UfUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 176) |
| tracrRNA | mA#mG#mC#mAmUmAmGmCmAmAmGfUrUmAr |
| 9 | AmAmArUmAmAmGmGrCrUmArGrUrCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 177) |
| tracrRNA | mA#mG#mC#mAmUmAmGmCmAmAmGrUfUmAr |
| 10 | AmAmArUmAmAmGmGrCrUmArGrUrCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 178) |
| tracrRNA | mA#mG#mC#mAmUmAmGmCmAmAmGrUrUmAf |
| 11 | AmAmArUmAmAmGmGrCrUmArGrUrCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 179) |
| tracrRNA | mA#mG#mC#mAmUmAmGmCmAmAmGrUrUmAr |
| 12 | AmAmAfUmAmAmGmGrCrUmArGrUrCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 180) |
| tracrRNA | mA#mG#mC#mAmUmAmGmCmAmAmGrUrUmAr |
| 13 | AmAmArUmAmAmGmGfCrUmArGrUrCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 181) |
| tracrRNA | mA#mG#mC#mAmUmAmGmCmAmAmGrUrUmAr |
| 14 | AmAmArUmAmAmGmGrCfUmArGrUrCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 182) |
| tracrRNA | mA#mG#mC#mAmUmAmGmCmAmAmGrUrUmAr |
| 15 | AmAmArUmAmAmGmGrCrUmAfGrUrCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 183) |
| tracrRNA | mA#mG#mC#mAmUmAmGmCmAmAmGrUrUmAr |
| 16 | AmAmArUmAmAmGmGrCrUmArGfUrCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 184) |
| tracrRNA | mA#mG#mC#mAmUmAmGmCmAmAmGrUrUmAr |
| 17 | AmAmArUmAmAmGmGrCrUmArGrUfCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 185) |
| tracrRNA | mA#mG#mC#mAmUmAmGmCmAmAmGrUrUmAr |
| 18 | AmAmArUmAmAmGmGrCrUmArGrUrCmCfGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 186) |
| tracrRNA | mA#mG#mC#mAmUmAmGmCmAmAmGrUrUmAr |
| 19 | AmAmArUmAmAmGmGrCrUmArGrUrCmCrGf |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 187) |
| tracrRNA | mA#mG#mC#mAmUmAmGmCmAmAmGrUrUmAr |
| 20 | AmAmArUmAmAmGmGrCrUmArGrUrCmCrGr |
| | UfUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 188) |
| tracrRNA | mA#mG#mC#mAmUmAmGmCmAmAmGrU#rU#m |
| 21 | ArA#mAmAfUmAmAmGmGfCfUmArG#fUfCm |
| | CrG#rU#rU#mAmUmCmAmAmCmUmUmGmAmA |
| | mAmAmAmGmUmGmGmCmAmCmCmGmAmGmUmC |
| | mGmGmUmGmC#mU#mU#mU |
| | (SEQ ID NO: 189) |
| tracrRNA | mA#mG#mC#mAmUmAmGmCmAmAmGmUrUmAr |
| 22 | AmAmArUmAmAmGmGrCrUmArGrUrCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 190) |
| tracrRNA | mA#mG#mC#mAmUmAmGmCmAmAmGrUmUmAr |
| 23 | AmAmArUmAmAmGmGrCrUmArGrUrCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 191) |
| tracrRNA | mA#mG#mC#mAmUmAmGmCmAmAmGrUrUmAm |
| 24 | AmAmArUmAmAmGmGrCrUmArGrUrCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 173) |
| tracrRNA | mA#mG#mC#mAmUmAmGmCmAmAmGrUrUmAr |
| 25 | AmAmAmUmAmAmGmGrCrUmArGrUrCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 192) |
| tracrRNA | mA#mG#mC#mAmUmAmGmCmAmAmGrUrUmAr |
| 26 | AmAmArUmAmAmGmGmCrUmArGrUrCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 193) |
| tracrRNA | mA#mG#mC#mAmUmAmGmCmAmAmGrUrUmAr |
| 27 | AmAmArUmAmAmGmGrCmUmArGrUrCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 194) |
| tracrRNA | mA#mG#mC#mAmUmAmGmCmAmAmGrUrUmAr |
| 28 | AmAmArUmAmAmGmGrCrUmAmGrUrCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 195) |
| tracrRNA | mA#mG#mC#mAmUmAmGmCmAmAmGrUrUmAr |
| 29 | AmAmArUmAmAmGmGrCrUmArGmUrCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 196) |
| tracrRNA | mA#mG#mC#mAmUmAmGmCmAmAmGrUrUmAr |
| 30 | AmAmArUmAmAmGmGrCrUmArGrUmCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 197) |
| tracrRNA | mA#mG#mC#mAmUmAmGmCmAmAmGrUrUmAr |
| 31 | AmAmArUmAmAmGmGrCrUmArGrUrCmCmGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 198) |
| tracrRNA | mA#mG#mC#mAmUmAmGmCmAmAmGrUrUmAr |
| 32 | AmAmArUmAmAmGmGrCrUmArGrUrCmCrGm |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 199) |
| tracrRNA | mA#mG#mC#mAmUmAmGmCmAmAmGrUrUmAr |
| 33 | AmAmArUmAmAmGmGrCrUmArGrUrCmCrGr |
| | UmUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 200) |
| tracrRNA | mA#mG#mC#mAmUmAmGmCmAmAmGrU#rUmA |
| 34 | rAmAmArUmAmAmGmGrCrUmArGrUrCmCrG |
| | rUrUmAmUmCmAmAmCmUmUmGmAmAmAmAmA |
| | mGmUmGmGmCmAmCmCmGmAmGmUmCmGmGmU |
| | mGmC#mU#mU#mU |
| | (SEQ ID NO: 201) |
| tracrRNA | mA#mG#mC#mAmUmAmGmCmAmAmGrUrU#mA |
| 35 | rAmAmArUmAmAmGmGrCrUmArGrUrCmCrG |
| | rUrUmAmUmCmAmAmCmUmUmGmAmAmAmAmA |
| | mGmUmGmGmCmAmCmCmGmAmGmUmCmGmGmU |
| | mGmC#mU#mU#mU |
| | (SEQ ID NO: 202) |
| tracrRNA | mA#mG#mC#mAmUmAmGmCmAmAmGrUrUmAr |
| 36 | A#mAmArUmAmAmGmGrCrUmArGrUrCmCrG |
| | rUrUmAmUmCmAmAmCmUmUmGmAmAmAmAmA |
| | mGmUmGmGmCmAmCmCmGmAmGmUmCmGmGmU |
| | mGmC#mU#mU#mU |
| | (SEQ ID NO: 203) |
| tracrRNA | mA#mG#mC#mAmUmAmGmCmAmAmGrUrUmAr |
| 37 | AmAmArU#mAmAmGmGrCrUmArGrUrCmCrG |
| | rUrUmAmUmCmAmAmCmUmUmGmAmAmAmAmA |
| | mGmUmGmGmCmAmCmCmGmAmGmUmCmGmGmU |
| | mGmC#mU#mU#mU |
| | (SEQ ID NO: 204) |
| tracrRNA | mA#mG#mC#mAmUmAmGmCmAmAmGrUrUmAr |
| 38 | AmAmArUmAmAmGmGrC#rUmArGrUrCmCrG |
| | rUrUmAmUmCmAmAmCmUmUmGmAmAmAmAmA |
| | mGmUmGmGmCmAmCmCmGmAmGmUmCmGmGmU |
| | mGmC#mU#mU#mU |
| | (SEQ ID NO: 205) |
| tracrRNA | mA#mG#mC#mAmUmAmGmCmAmAmGrUrUmAr |
| 39 | AmAmArUmAmAmGmGrCrU#mArGrUrCmCrG |
| | rUrUmAmUmCmAmAmCmUmUmGmAmAmAmAmA |
| | mGmUmGmGmCmAmCmCmGmAmGmUmCmGmGmU |
| | mGmC#mU#mU#mU |
| | (SEQ ID NO: 206) |
| tracrRNA | mA#mG#mC#mAmUmAmGmCmAmAmGrUrUmAr |
| 40 | AmAmArUmAmAmGmGrCrUmArG#rUrCmCrG |
| | rUrUmAmUmCmAmAmCmUmUmGmAmAmAmAmA |
| | mGmUmGmGmCmAmCmCmGmAmGmUmCmGmGmU |
| | mGmC#mU#mU#mU |
| | (SEQ ID NO: 207) |
| tracrRNA | mA#mG#mC#mAmUmAmGmCmAmAmGrUrUmAr |
| 41 | AmAmArUmAmAmGmGrCrUmArGrU#rCmCrG |
| | rUrUmAmUmCmAmAmCmUmUmGmAmAmAmAmA |
| | mGmUmGmGmCmAmCmCmGmAmGmUmCmGmGmU |
| | mGmC#mU#mU#mU |
| | (SEQ ID NO: 208) |
| tracrRNA | mA#mG#mC#mAmUmAmGmCmAmAmGrUrUmAr |
| 42 | AmAmArUmAmAmGmGrCrUmArGrUrC#mCrG |
| | rUrUmAmUmCmAmAmCmUmUmGmAmAmAmAmA |
| | mGmUmGmGmCmAmCmCmGmAmGmUmCmGmGmU |
| | mGmC#mU#mU#mU |
| | (SEQ ID NO: 209) |
| tracrRNA | mA#mG#mC#mAmUmAmGmCmAmAmGrUrUmAr |
| 43 | AmAmArUmAmAmGmGrCrUmArGrUrCmCrG# |
| | rUrUmAmUmCmAmAmCmUmUmGmAmAmAmAmA |
| | mGmUmGmGmCmAmCmCmGmAmGmUmCmGmGmU |
| | mGmC#mU#mU#mU |
| | (SEQ ID NO: 210) |
| tracrRNA | mA#mG#mC#mAmUmAmGmCmAmAmGrUrUmAr |
| 44 | AmAmArUmAmAmGmGrCrUmArGrUrCmCrGr |
| | U#rUmAmUmCmAmAmCmUmUmGmAmAmAmAmA |
| | mGmUmGmGmCmAmCmCmGmAmGmUmCmGmGmU |
| | mGmC#mU#mU#mU |
| | (SEQ ID NO: 211) |
| tracrRNA | mA#mG#mC#mAmUmAmGmCmAmAmGrUrUmAr |
| 45 | AmAmArUmAmAmGmGrCrUmArGrUrCmCrGr |
| | UrU#mAmUmCmAmAmCmUmUmGmAmAmAmAmA |
| | mGmUmGmGmCmAmCmCmGmAmGmUmCmGmGmU |
| | mGmC#mU#mU#mU |
| | (SEQ ID NO: 212) |
| tracrRNA | mA#mG#mC#mAmUmAmGmCmAmAmGfUfUmAr |
| 46 | AmAmArUmAmAmGmGrCrUmArGrUrCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 213) |
| tracrRNA | mA#mG#mC#mAmUmAmGmCmAmAmGrUrUmAf |
| 47 | AmAmAmUmAmAmGmGrCrUmArGrUrCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 214) |
| tracrRNA | mA#mG#mC#mAmUmAmGmCmAmAmGfUfUmAf |
| 48 | AmAmAfUmAmAmGmGrCrUmArGrUrCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 215) |
| tracrRNA | mA#mG#mC#mAmUmAmGmCmAmAmGrUrUmAr |
| 49 | AmAmArUmAmAmGmGfCfUmArGrUrCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 216) |
| tracrRNA | mA#mG#mC#mAmUmAmGmCmAmAmGrUrUmAr |
| 50 | AmAmArUmAmAmGmGrCrUmAfGfUfCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 217) |
| tracrRNA | mA#mG#mC#mAmUmAmGmCmAmAmGrUrUmAr |
| 51 | AmAmArUmAmAmGmGrCrUmArGrUrCmCfGf |
| | UfUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 218) |
| tracrRNA | mA#mG#mC#mAmUmAmGmCmAmAmGrUrUmAr |
| 52 | AmAmArUmAmAmGmGfCfUmArGrUrCmCfGf |
| | UfUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 219) |
| tracrRNA | mA#mG#mC#mAmUmAmGmCmAmAmGrUrUmAr |
| 53 | AmAmArUmAmAmGmGfCfUmAfGfUfCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 220) |
| tracrRNA | mA#mG#mC#mAmUmAmGmCmAmAmGrUrUmAr |
| 54 | AmAmArUmAmAmGmGrCrUmAfGfUfCmCfGf |
| | UfUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 221) |
| tracrRNA | mA#mG#mC#mAmUmAmGmCmAmAmGrUrUmAr |
| 55 | AmAmArUmAmAmGmGrCrUmAfGfUfCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#Mu |
| | (SEQ ID NO: 217) |
| tracrRNA | mA#mG#mC#mAmUmAmGmCmAmAmGrUrUmAr |
| 56 | AmAmArUmAmAmGmGrCrUmArGrUrCmCfGr |
| | UfUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 222) |
| tracrRNA | mA#mG#mC#mAmUmAmGmCmAmAmGrUrUmAr |
| 57 | AmAmArUmAmAmGmGfCfUmAfGrUfCmCfGr |
| | UfUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 223) |
| tracrRNA | mA#mG#mC#mAmUmAmGmCmAmAmGrUrUmAr |
| 58 | AmAmArUmAmAmGmGrCrUmAfGrUfCmCfGr |
| | UfUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 224) |
| tracrRNA | mA#mG#mC#mAmUmAmGmCmAmAmGrUrUmAr |
| 59 | AmAmArUmAmAmGmGfCrUmAfGrUfCmCfGr |
| | UfUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 225) |
| tracrRNA | mA#mG#mC#mAmUmAmGmCmAmAmGmUmUmAr |
| 60 | AmAmArUmAmAmGmGrCrUmArGrUrCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#m |
| | U(SEQ ID NO: 226) |
| tracrRNA | mA#mG#mC#mAmUmAmGmCmAmAmGrUrUmAm |
| 61 | AmAmAmUmAmAmGmGrCrUmArGrUrCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 227) |
| tracrRNA | mA#mG#mC#mAmUmAmGmCmAmAmGmUmUmAm |
| 62 | AmAmAmUmAmAmGmGrCrUmArGrUrCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 228) |
| tracrRNA | mA#mG#mC#mAmUmAmGmCmAmAmGrUrUmAr |
| 63 | AmAmArUmAmAmGmGmCmUmArGrUrCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 229) |
| tracrRNA | mA#mG#mC#mAmUmAmGmCmAmAmGrUrUmAr |
| 64 | AmAmArUmAmAmGmGrCrUmAmGmUmCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 230) |
| tracrRNA | mA#mG#mC#mAmUmAmGmCmAmAmGrUrUmAr |
| 65 | AmAmArUmAmAmGmGrCrUmArGrUrCmCmGm |
| | UmUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 231) |
| tracrRNA | mA#mG#mC#mAmUmAmGmCmAmAmGrUrUmAr |
| 66 | AmAmArUmAmAmGmGmCmUmArGrUrCmCmGm |
| | UmUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 232) |
| tracrRNA | mA#mG#mC#mAmUmAmGmCmAmAmGrUrUmAr |
| 67 | AmAmArUmAmAmGmGmCmUmAmGmUmCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 233) |
| tracrRNA | mA#mG#mC#mAmUmAmGmCmAmAmGrUrUmAr |
| 68 | AmAmArUmAmAmGmGrCrUmAmGmUmCmCmGm |
| | UmUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 234) |
| tracrRNA | mA#mG#mC#mAmUmAmGmCmAmAmGrUrUmAr |
| 69 | AmAmArUmAmAmGmGrCrUmAmGrUmCmCrGr |
| | UUrmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 235) |
| tracrRNA | mA#mG#mC#mAmUmAmGmCmAmAmGrUrUmAr |
| 70 | AmAmArUmAmAmGmGrCrUmArGrUrCmCmGr |
| | UmUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 236) |
| tracrRNA | mA#mG#mC#mAmUmAmGmCmAmAmGrUrUmAr |
| 71 | AmAmArUmAmAmGmGmCmUmAmGrUmCmCmGr |
| | UmUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 237) |
| tracrRNA | mA#mG#mC#mAmUmAmGmCmAmAmGrUrUmAr |
| 72 | AmAmArUmAmAmGmGrCrUmAmGrUmCmCmGr |
| | UmUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 238) |
| tracrRNA | mA#mG#mC#mAmUmAmGmCmAmAmGrUrUmAr |
| 73 | AmAmArUmAmAmGmGmCrUmAmGrUmCmCmGr |
| | UmUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 239) |
| tracrRNA | mA#mG#mC#mAmUmAmGmCmAmAmGdUdUmAr |
| 74 | AmAmArUmAmAmGmGrCrUmArGrUrCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 240) |
| tracrRNA | mA#mG#mC#mAmUmAmGmCmAmAmGrUrUmAd |
| 75 | AmAmAdUmAmAmGmGrCrUmArGrUrCmCrGr |
| | UrUmAmUmCmAmAmCmUmUmGmAmAmAmAmAm |
| | mGUmGmGmCmAmCmCmGmAmGmUmCmGmGmUm |
| | GmC#mU#mU#mU |
| | (SEQ ID NO: 241) |
| tracrRNA | mA#mG#mC#mAmUmAmGmCmAmAmGdUdUmAd |
| 76 | AmAmAdUmAmAmGmGrCrUmArGrUrCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 242) |
| tracrRNA | mA#mG#mC#mAmUmAmGmCmAmAmGrUrUmAr |
| 77 | AmAmArUmAmAmGmGdCdUmArGrUrCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 243) |
| tracrRNA | mA#mG#mC#mAmUmAmGmCmAmAmGrUrUmAr |
| 78 | AmAmArUmAmAmGmGrCrUmAdGdUdCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 244) |
| tracrRNA | mA#mG#mC#mAmUmAmGmCmAmAmGrUrUmAr |
| 79 | AmAmArUmAmAmGmGrCrUmArGrUrCmCdGd |
| | UdUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 245) |
| tracrRNA | mA#mG#mC#mAmUmAmGmCmAmAmGrUrUmAr |
| 80 | AmAmArUmAmAmGmGdCdUmArGrUrCmCdGd |
| | UdUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 246) |
| tracrRNA | mA#mG#mC#mAmUmAmGmCmAmAmGrUrUmAr |
| 81 | AmAmArUmAmAmGmGdCdUmAdGdUdCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 247) |
| tracrRNA | mA#mG#mC#mAmUmAmGmCmAmAmGrUrUmAr |
| 82 | AmAmArUmAmAmGmGrCrUmAdGdUdCmCdGd |
| | UdUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 248) |
| tracrRNA | mA#mG#mC#mAmUmAmGmCmAmAmGrUrUmAr |
| 83 | AmAmArUmAmAmGmGrCrUmAdGrUdCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 249) |
| tracrRNA | mA#mG#mC#mAmUmAmGmCmAmAmGrUrUmAr |
| 84 | AmAmArUmAmAmGmGrCrUmArGrUrCmCdGr |
| | UdUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 250) |
| tracrRNA | mA#mG#mC#mAmUmAmGmCmAmAmGrUrUmAr |
| 85 | AmAmArUmAmAmGmGdCdUmAdGrUdCmCdGr |
| | UdUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 251) |
| tracrRNA | mA#mG#mC#mAmUmAmGmCmAmAmGrUrUmAr |
| 86 | AmAmArUmAmAmGmGrCrUmAdGrUdCmCdGr |
| | UdUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 252) |
| tracrRNA | mA#mG#mC#mAmUmAmGmCmAmAmGrUrUmAr |
| 87 | AmAmArUmAmAmGmGdCrUmAdGrUdCmCdGr |
| | UdUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 253) |
| tracrRNA | mA#mG#mC#mAmUmAmGmCmAmAmGrU#rU#m |
| 88 | ArAmAmArUmAmAmGmGrCrUmArGrUrCmCr |
| | GrUrUmAmUmCmAmAmCmUmUmGmAmAmAmAm |
| | AmGmUmGmGmCmAmCmCmGmAmGmUmCmGmGm |
| | UmGmC#mU#mU#mU |
| | (SEQ ID NO: 254) |
| tracrRNA | mA#mG#mC#mAmUmAmGmCmAmAmGrUrUmAr |
| 89 | A#mAmArU#mAmAmGmGrCrUmArGrUrCmCr |
| | GrUrUmAmUmCmAmAmCmUmUmGmAmAmAmAm |
| | AmGmUmGmGmCmAmCmCmGmAmGmUmCmGmGm |
| | UmGmC#mU#mU#mU |
| | (SEQ ID NO: 255) |
| tracrRNA | mA#mG#mC#mAmUmAmGmCmAmAmGrU#rU#m |
| 90 | ArA#mAmArU#mAmAmGmGrCrUmArGrUrCm |
| | CrGrUrUmAmUmCmAmAmCmUmUmGmAmAmAm |
| | AmAmGmUmGmGmCmAmCmCmGmAmGmUmCmGm |
| | GmUmGmC#mU#mU#mU |
| | (SEQ ID NO: 256) |
| tracrRNA | mA#mG#mC#mAmUmAmGmCmAmAmGrUrUmAr |
| 91 | AmAmArUmAmAmGmGrC#rU#mArGrUrCmCr |
| | GrUrUmAmUmCmAmAmCmUmUmGmAmAmAmAm |
| | AmGmUmGmGmCmAmCmCmGmAmGmUmCmGmGm |
| | UmGmC#mU#mU#mU |
| | (SEQ ID NO: 257) |
| tracrRNA | mA#mG#mC#mAmUmAmGmCmAmAmGrUrUmAr |
| 92 | AmAmArUmAmAmGmGrCrUmArG#rU#rC#mC |
| | rGrUrUmAmUmCmAmAmCmUmUmGmAmAmAmA |
| | mAmGmUmGmGmCmAmCmCmGmAmGmUmCmGmG |
| | mUmGmC#mU#mU#mU |
| | (SEQ ID NO: 258) |
| tracrRNA | mA#mG#mC#mAmUmAmGmCmAmAmGrUrUmAr |
| 93 | AmAmArUmAmAmGmGrCrUmArGrUrCmCrG# |
| | rU#rU#mAmUmCmAmAmCmUmUmGmAmAmAmA |
| | mAmGmUmGmGmCmAmCmCmGmAmGmUmCmGmG |
| | mUmGmC#mU#mU#mU |
| | (SEQ ID NO: 259) |
| tracrRNA | mA#mG#mC#mAmUmAmGmCmAmAmGrUrUmAr |
| 94 | AmAmArUmAmAmGmGrC#rU#mArGrUrCmCr |
| | G#rU#rU#mAmUmCmAmAmCmUmUmGmAmAmA |
| | mAmAmGmUmGmGmCmAmCmCmGmAmGmUmCmG |
| | mGmUmGmC#mU#mU#mU |
| | (SEQ ID NO: 260) |
| tracrRNA | mA#mG#mC#mAmUmAmGmCmAmAmGrUrUmAr |
| 95 | AmAmArUmAmAmGmGrC#rU#mArG#rU#rC# |
| | mCrGrUrUmAmUmCmAmAmCmUmUmGmAmAmA |
| | mAmAmGmUmGmGmCmAmCmCmGmAmGmUmCmG |
| | mGmUmGmC#mU#mU#mU |
| | (SEQ ID NO: 261) |
| tracrRNA | mA#mG#mC#mAmUmAmGmCmAmAmGrUrUmAr |
| 96 | AmAmArUmAmAmGmGrCrUmArG#rU#rC#mC |
| | rG#rU#rU#mAmUmCmAmAmCmUmUmGmAmAm |
| | AmAmAmGmUmGmGmCmAmCmCmGmAmGmUmCm |
| | GmGmUmGmC#mU#mU#mU |
| | (SEQ ID NO: 262) |
| tracrRNA | mA#mG#mC#mAmUmAmGmCmAmAmGrUrUmAr |
| 97 | AmAmArUmAmAmGmGrCrUmArG#rUrC#mCr |
| | GrUrUmAmUmCmAmAmCmUmUmGmAmAmAmAm |
| | AmGmUmGmGmCmAmCmCmGmAmGmUmCmGmGm |
| | UmGmC#mU#mU#mU |
| | (SEQ ID NO: 263) |
| tracrRNA | mA#mG#mC#mAmUmAmGmCmAmAmGrUrUmAr |
| 98 | AmAmArUmAmAmGmGrCrUmArGrUrCmCrG# |
| | rUrU#mAmUmCmAmAmCmUmUmGmAmAmAmAm |
| | AmGmUmGmGmCmAmCmCmGmAmGmUmCmGmGm |
| | UmGmC#mU#mU#mU |
| | (SEQ ID NO: 264) |
| tracrRNA | mA#mG#mC#mAmUmAmGmCmAmAmGrUrUmAr |
| 99 | AmAmArUmAmAmGmGrC#rU#mArG#rU#rCm |
| | CrG#rUrU#mAmUmCmAmAmCmUmUmGmAmAm |
| | AmAmAmGmUmGmGmCmAmCmCmGmAmGmUmCm |
| | GmGmUmGmC#mU#mU#mU |
| | (SEQ ID NO: 265) |
| tracrRNA | mA#mG#mC#mAmUmAmGmCmAmAmGrUrUmAr |
| 100 | AmAmArUmAmAmGmGrCrUmArG#rU#rCmCr |
| | G#rUrU#mAmUmCmAmAmCmUmUmGmAmAmAm |
| | AmAmGmUmGmGmCmAmCmCmGmAmGmUmCmGm |
| | GmUmGmC#mU#mU#mU |
| | (SEQ ID NO: 266) |
| tracrRNA | mA#mG#mC#mAmUmAmGmCmAmAmGrUrUmAr |
| 101 | AmAmArUmAmAmGmGrC#rUmArG#rU#rCmC |
| | rG#rUrU#mAmUmCmAmAmCmUmUmGmAmAmA |
| | mAmAmGmUmGmGmCmAmCmCmGmAmGmUmCmG |
| | mGmUmGmC#mU#mU#mU |
| | (SEQ ID NO: 267) |
| tracrRNA | mA#mG#mC#mAmUmAmGmCmAmAmGrUrUmAr |
| 104 | AmAmAdUmAmAmGmGdCdUmArGdUdCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 268) |
| tracrRNA | mA#mG#mC#mAmUmAmGmCmAmAmGrUdUmAr |
| 105 | AmAmAdUmAmAmGmGdCdUmAdGdUdCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 269) |
| tracrRNA | mA#mG#mC#mAmUmAmGmCmAmAmGdUdUmAd |
| 106 | AmAmAdUmAmAmGmGdCdUmAdGdUdCmCdGd |
| | UdUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 270) |
| tracrRNA | mA#mG#mC#mAmUmAmGmCmAmAmGsUsUmAr |
| 107 | AmAmAsUmAmAmGmGrCsUmArGsUrCmCrGs |
| | UsUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 271) |
| tracrRNA | mA#mG#mC#mAmUmAmGmCmAmAmGsUsUmAr |
| 108 | AmAmAsUmAmAmGmGrCrUmArGrUrCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 272) |
| tracrRNA | mA#mG#mC#mAmUmAmGmCmAmAmGrUrUmAr |
| 109 | AmAmArUmAmAmGmGrCsUmArGsUrCmCrGs |
| | UsUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 273) |
| tracrRNA | mA#mG#mC#mAmUmAmGmCmAmAmGsUrUmAr |
| 110 | AmAmArUmAmAmGmGrCrUmArGrUrCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 274) |
| tracrRNA | mA#mG#mC#mAmUmAmGmCmAmAmGrUsUmAr |
| 111 | AmAmArUmAmAmGmGrCrUmArGrUrCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 275) |
| tracrRNA | mA#mG#mC#mAmUmAmGmCmAmAmGrUrUmAr |
| 112 | AmAmAsUmAmAmGmGrCrUmArGrUrCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 276) |
| tracrRNA | mA#mG#mC#mAmUmAmGmCmAmAmGrUrUmAr |
| 113 | AmAmArUmAmAmGmGrCsUmArGrUrCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 277) |
| tracrRNA | mA#mG#mC#mAmUmAmGmCmAmAmGrUrUmAr |
| 114 | AmAmArUmAmAmGmGrCrUmArGsUrCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 278) |
| tracrRNA | mA#mG#mC#mAmUmAmGmCmAmAmGrUrUmAr |
| 115 | AmAmArUmAmAmGmGrCrUmArGrUrCmCrGs |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 279) |
| tracrRNA | mA#mG#mC#mAmUmAmGmCmAmAmGrUrUmAr |
| 116 | AmAmArUmAmAmGmGrCrUmArGrUrCmCrGr |
| | UsUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 280) |

### ExNA-Modified Guide RNAs

Described herein are crRNAs and tracrRNAs containing at least one of the backbone modification Extended Nucleic Acid (exNA). This chemical modification of the backbone significantly enhances oligonucleotide metabolic stability. The chemical modification includes one or more carbon atoms or chains inserted in the backbone at the 5′-position, 3′-position, or both. This structural modulation forms non-canonical stretched/flexible structure on oligo-backbones, which protect oligonucleotides from cleavage by various nucleases.

The synthesis protocol and structure for exNA-modified oligonucleotides is described in WO2021242883, WO2021195533, and WO2020198509, each of which is incorporated herein by reference. Importantly, the exNA monomer phosphoramidite synthesis can be realized from commercially available nucleosides and the exNA-modified oligonucleotide can be made using conventional oligonucleotide solid phase synthesis procedures on an automatic oligo synthesizer.

This synthetic procedure provides following noteworthy benefits. For example, the conversion of a regular nucleoside to an “exNA-format” is applicable to many diverse modified nucleosides. Thus, this expands the possibilities to synthesize and create many more types of modified oligonucleotides with compatibility of the chemical synthesis. Secondly, there is no need of a separate specific synthesis procedure during an oligonucleotide synthesis cycle. This is a huge benefit in the ease of use of these oligos, especially with an automated synthesizer where a bottle of exNA phosphoramidite could easily be added to the machine. Thirdly, there is no need of a specific oligonucleotide deprotection condition because the exNA phosphoramidites and oligos are compatible with conventional deprotection conditions. Again, this is beneficial for the ease of synthesis and in the use of an automated synthesizer. Fourthly, it is possible to synthesize mix-mer oligonucleotide having both exNA and clinically validated modified nucleotides (e.g., 2′-OMe, 2′-F, phosphorothioate, various ligand conjugates, lipid conjugates, etc.).

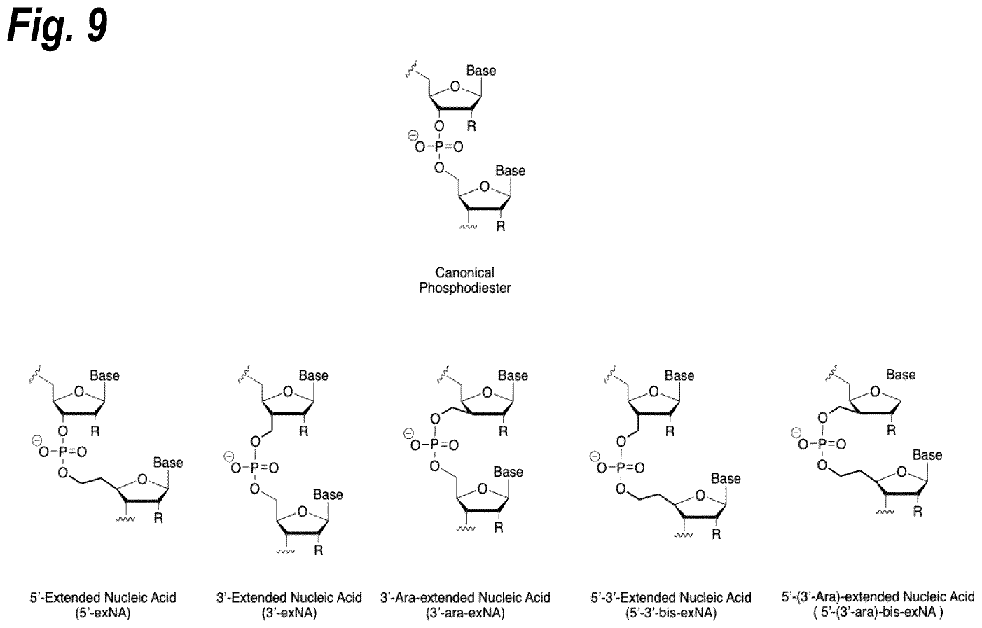

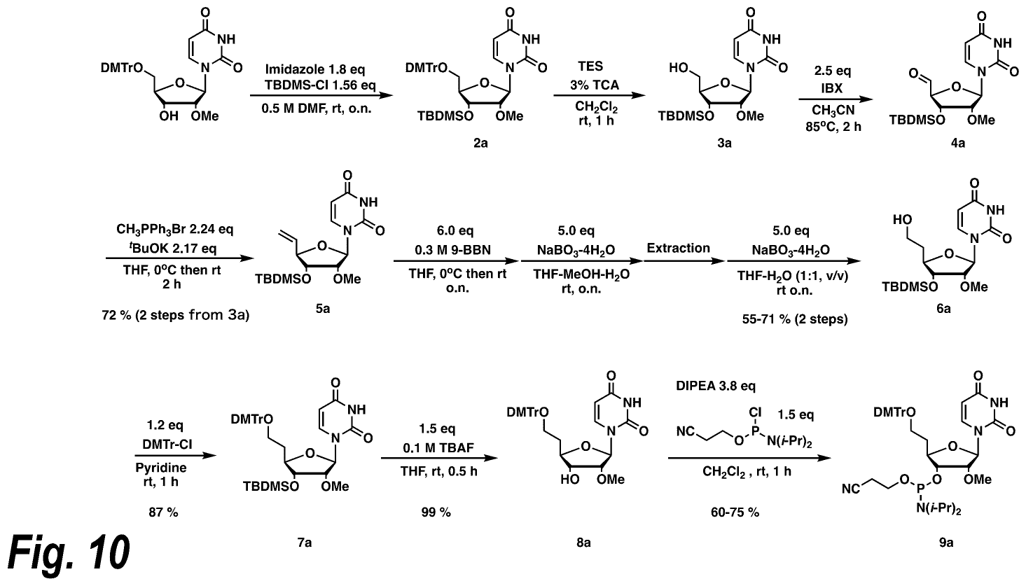

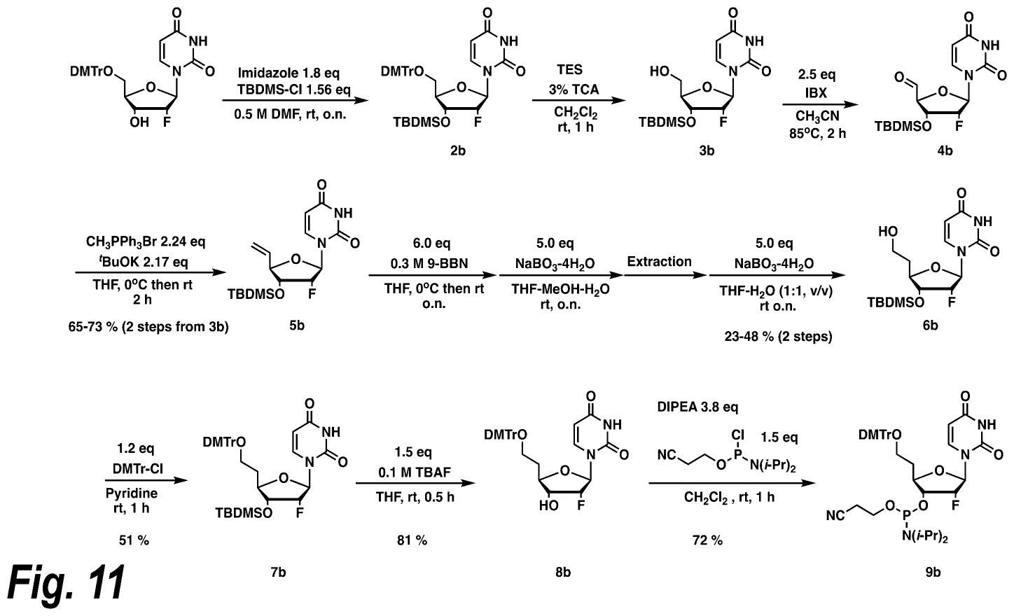

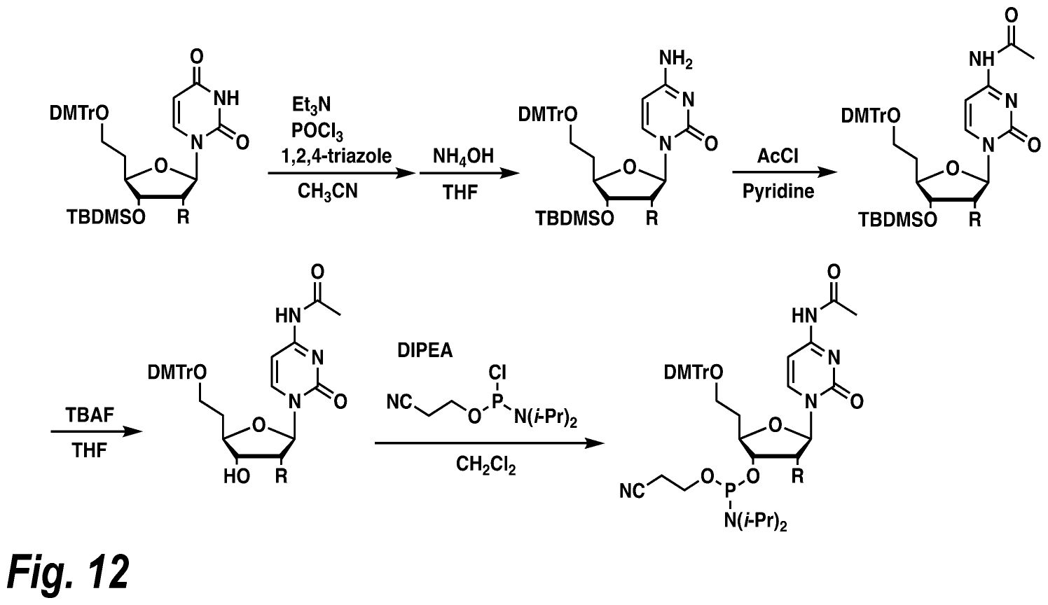

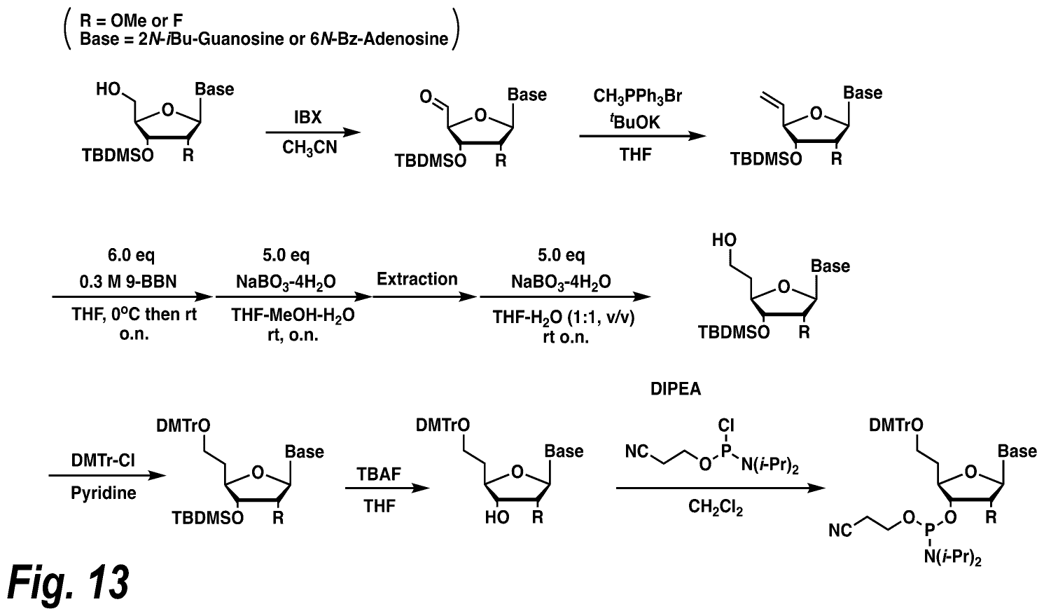

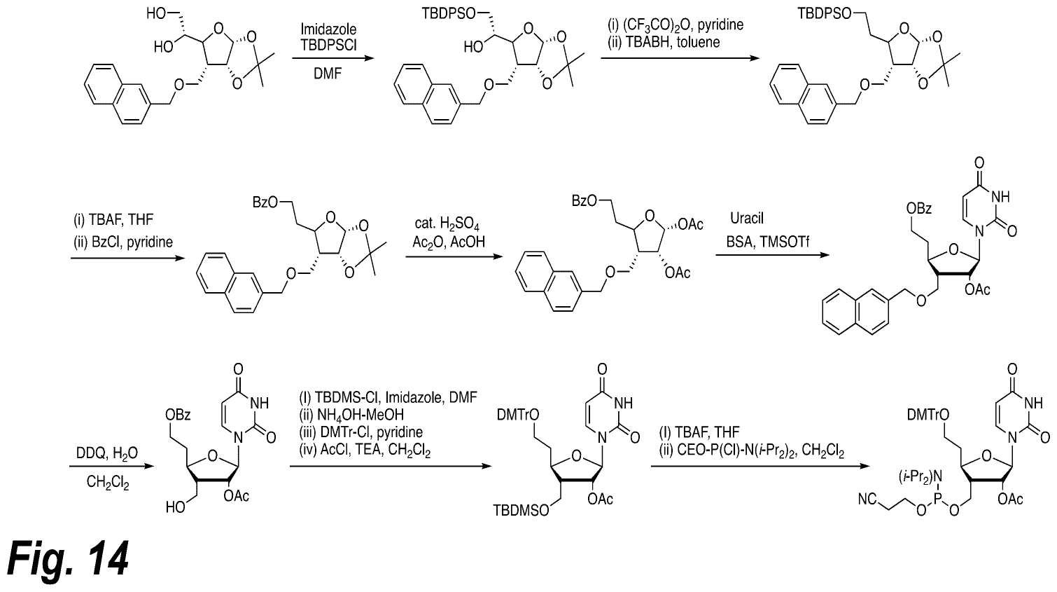

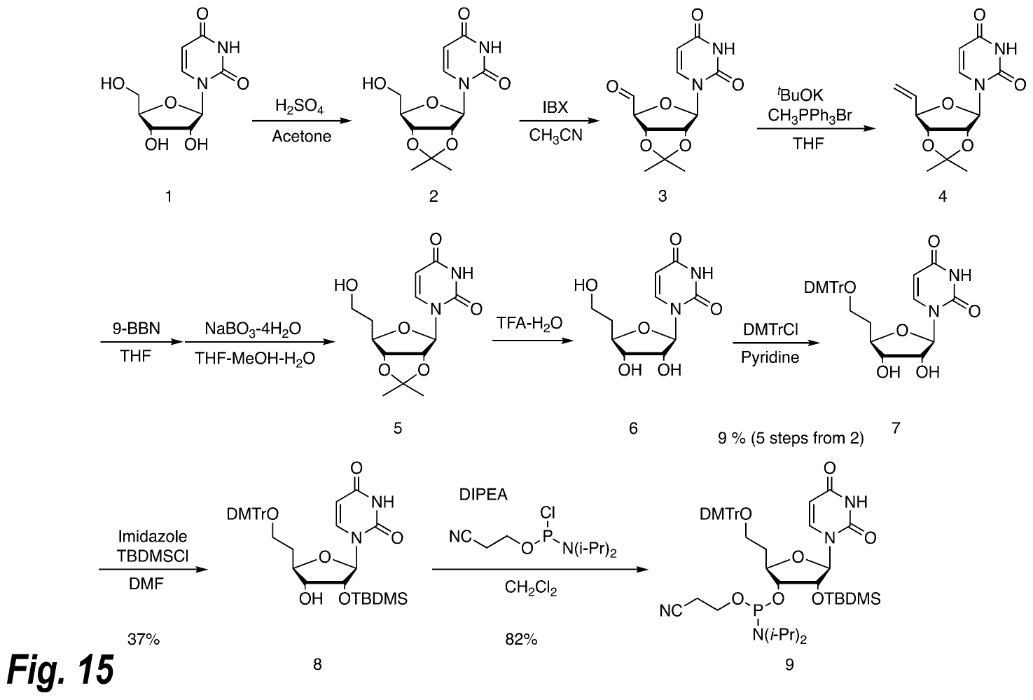

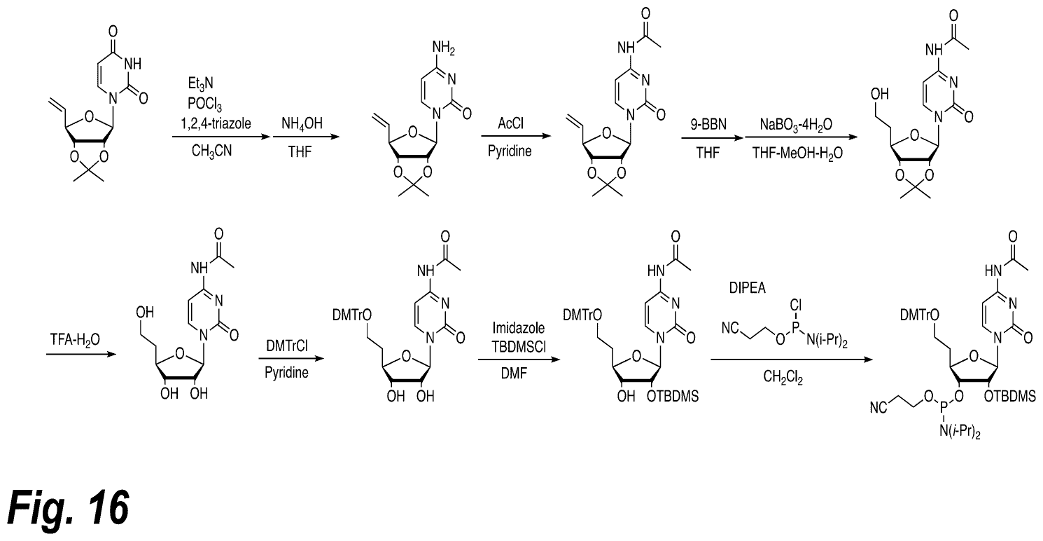

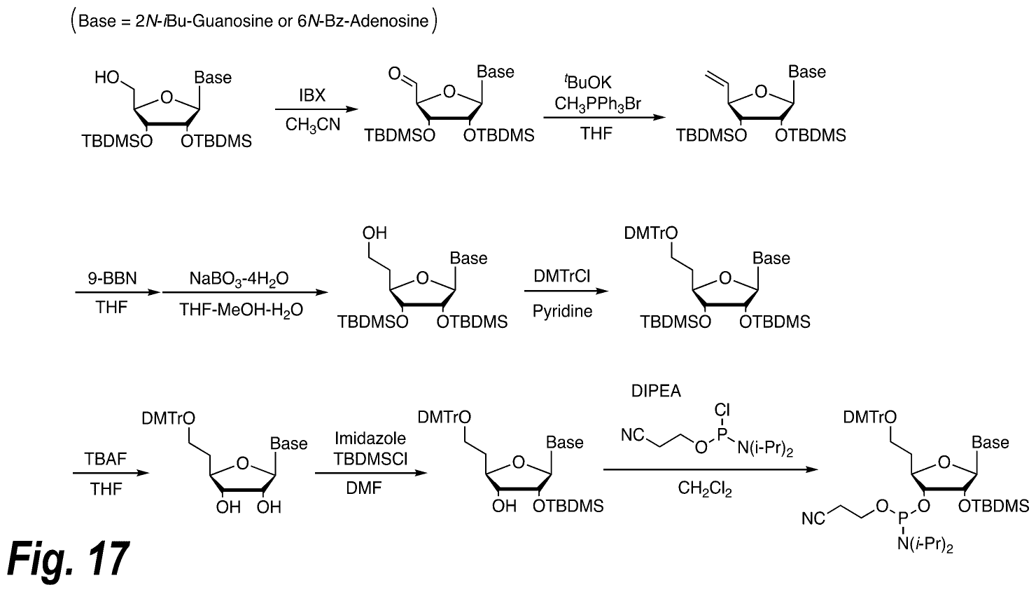

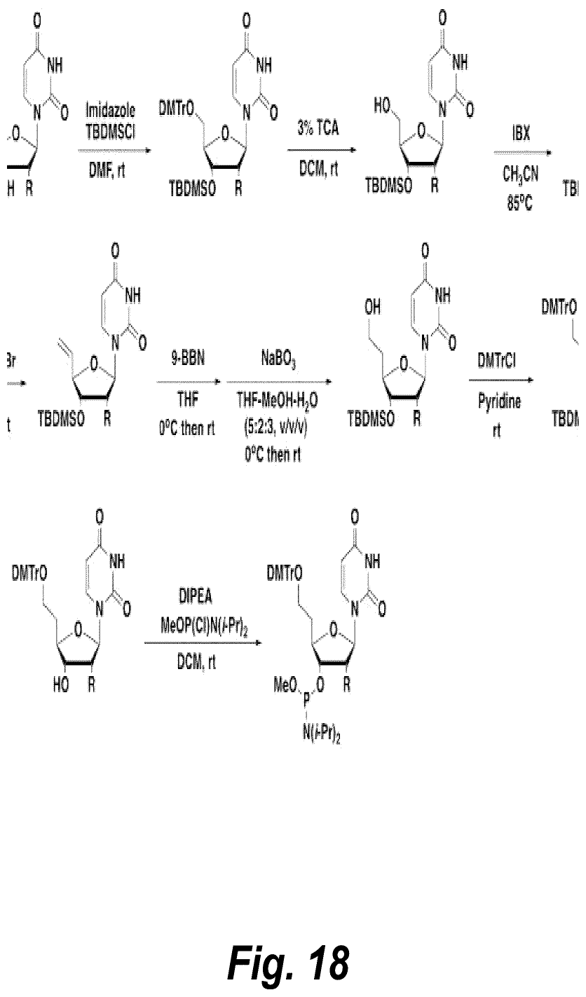

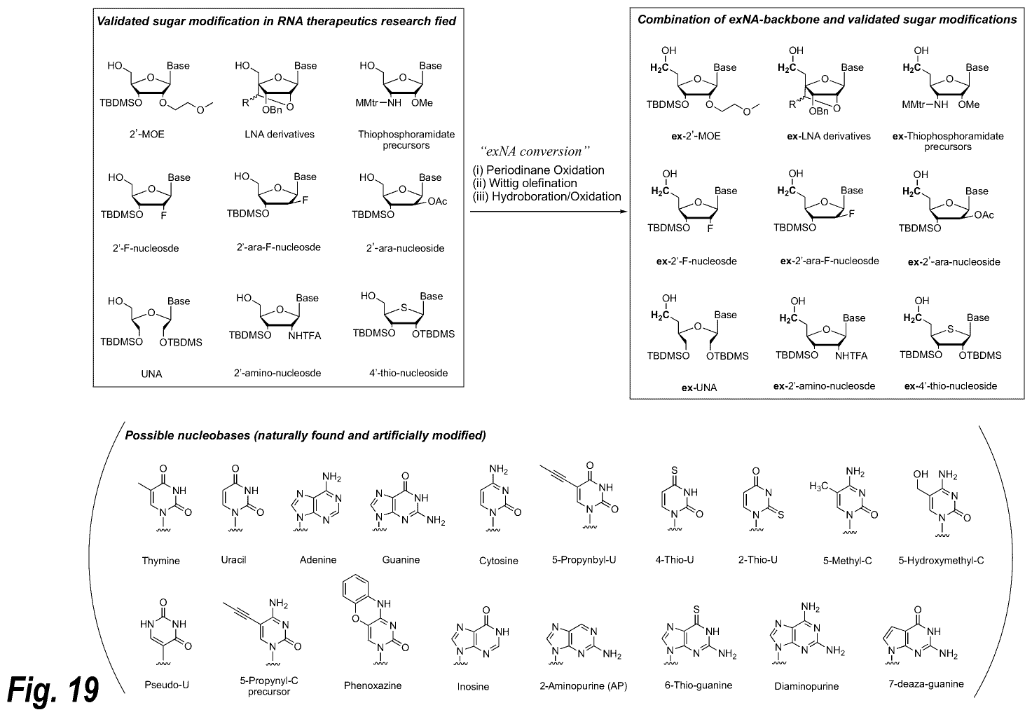

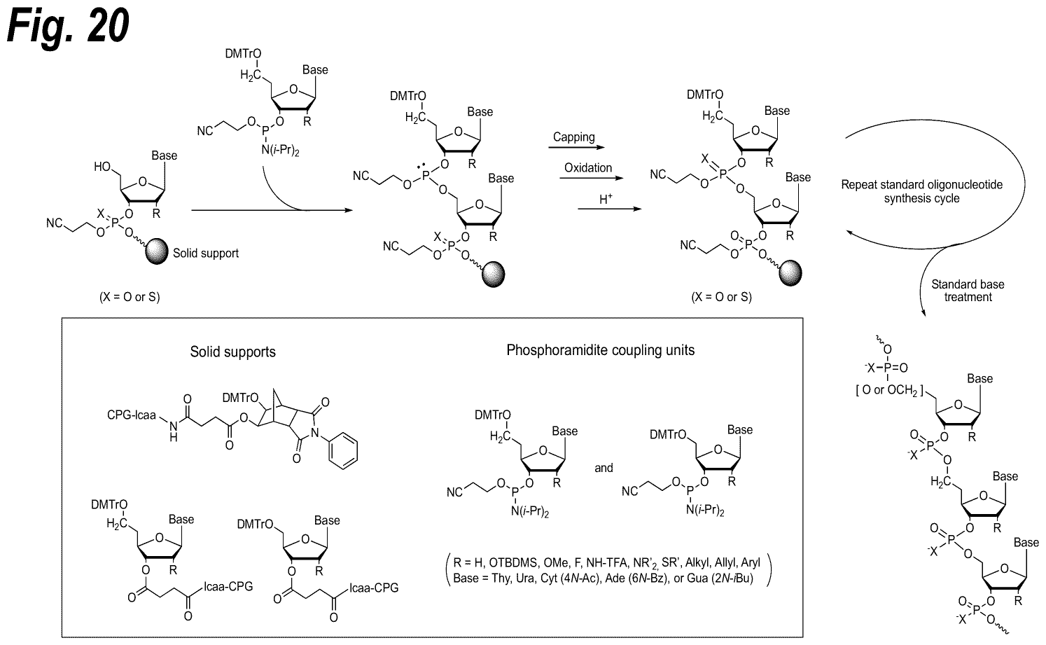

FIG. 9-FIG. 20 depict exemplary exNA internucleotide modifications as well as exemplary synthesis schemes.

In one aspect, the disclosure provides a crRNA and/or a tracrRNA comprising at least one modified intersubunit linkage of Formula Ia:

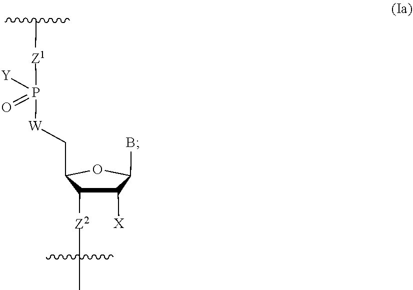

wherein:
B is a base moiety;
W is O or O(CH₂)ₙ¹, wherein n¹ is 1 to 10;
X is selected from the group consisting of H, OH, OR¹, R¹, F, Cl, Br, I, SH, SR¹, NH₂, NHR¹, NR¹₂, and COOR¹;
Y is selected from the group consisting of O⁻, OH, OR², NH⁻, NH₂, NR²₂, BH₃, S⁻, R², and SH;
Z¹ is O or O(CH₂)n² wherein n² is 1 to 10;
Z² is O or O(CH₂)n³ wherein n³ is 1 to 10;
R¹ is a substituted or unsubstituted C₁-C₆ alkyl, alkenyl, alkynyl, aryl, or mixtures thereof; and
R² is a substituted or unsubstituted C₁-C₆ alkyl, alkenyl, alkynyl, aryl, or mixtures thereof.

In an embodiment of Formula Ia, Z₁ is O(CH₂)ₙ², wherein n² is 1 to 10 and W is O. In an embodiment of Formula Ia, Z₁ is O and Wis O(CH₂)ₙ¹, wherein n¹ is 1 to 10. In an embodiment of Formula Ia, Z₁ is O(CH₂)ₙ², wherein n² is 1 to 10 and W is O(CH₂)ₙ¹, wherein n¹ is 1 to 10.

In an embodiment of Formula Ia, Z¹ is O(CH₂)ₙ², n² is 1, W is O, and Y is O⁻. In an embodiment of Formula Ia, Z¹ is O, W is O(CH₂)ₙ¹, n¹ is 1, and Y is O⁻. In an embodiment of Formula Ia, Z¹ is O(CH₂)ₙ², n² is 1, W is O, and Y is O⁻. In an embodiment of Formula Ia, Z¹ is O(CH₂)ₙ², n² is 1, W is O(CH₂)ₙ¹, and Y is O⁻. In an embodiment of Formula Ia, Z¹ is O(CH₂)ₙ², n² is 1, W is O(CH₂)ₙ¹, and Y is S⁻.

In an embodiment of Formula Ia, Y is S⁻. In an embodiment of Formula Ia, X is OR¹ or F.

In an embodiment of Formula Ia, the base moiety B is selected from the group consisting of adenine, guanine, cytosine, and uracil.

In one aspect, the disclosure provides a crRNA and/or a tracrRNA comprising at least one modified intersubunit linkage of Formula I:

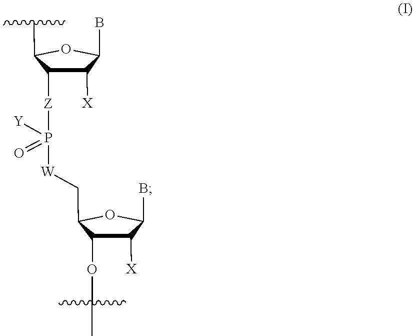

wherein:
B is a base moiety;
W is O or O(CH₂)ₙ¹, wherein n¹ is 1 to 10;
X is selected from the group consisting of H, OH, OR¹, R¹, F, Cl, Br, I, SH, SR¹, NH₂, NHR¹, NR¹₂, and COOR¹;
Y is selected from the group consisting of O⁻, OH, OR², NH , NH₂, NR²₂, BH₃, S⁻, R², and SH;
Z is O or O(CH₂)ₙ² wherein n² is 1 to 10;
R¹ is a substituted or unsubstituted C₁-C₆ alkyl, alkenyl, alkynyl, aryl, or mixtures thereof; and
R² is a substituted or unsubstituted C₁-C₆ alkyl, alkenyl, alkynyl, aryl, or mixtures thereof.

In an embodiment of Formula I, Z is O(CH₂)ₙ², wherein n² is 1 to 10 and W is O. In an embodiment of Formula I, Z is O and W is O(CH₂)ₙ¹, wherein n¹ is 1 to 10. In an embodiment of Formula I, Z is O(CH₂)»2, wherein n² is 1 to 10 and W is O(CH₂)ₙ¹, wherein n¹ is 1 to 10.

In an embodiment of Formula I, Z is O(CH₂)ₙ², n² is 1, W is O, and Y is O⁻. In an embodiment of Formula I, Z is O, W is O(CH₂)ₙ¹, n¹ is 1, and Y is O⁻. In an embodiment of Formula I, Z is O(CH₂)ₙ², n² is 1, W is O, and Y is O⁻. In an embodiment of Formula I, Z is O(CH₂)ₙ², n² is 1, W is O(CH₂)ₙ¹, and Y is O⁻. In an embodiment of Formula I, Z is O(CH₂)ₙ², n² is 1, W is O(CH₂)ₙ¹, and Y is S⁻.

In an embodiment of Formula I, Y is S⁻. In an embodiment of Formula I, X is OR¹ or F.

In an embodiment of Formula I, the base moiety B is selected from the group consisting of adenine, guanine, cytosine, and uracil.

In one aspect, the disclosure provides a crRNA and/or a tracrRNA comprising at least one modified intersubunit linkage of Formula IIa:

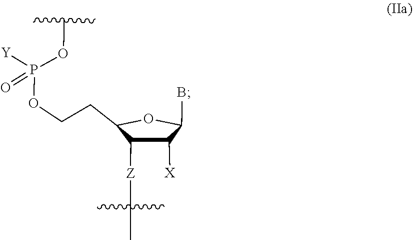

wherein:
B is a base moiety;
X is selected from the group consisting of H, OH, OR¹, R¹, F, Cl, Br, I, SH, SR^{′}, NH₂, NHR¹, NR¹₂, and COOR¹;
Y is selected from the group consisting of O⁻, OH, OR², NH⁻, NH₂, NR²₂, BH₃, S⁻, R², and SH;
Z is O or O(CH₂)ₙ² wherein n² is 1 to 10;
R¹ is a substituted or unsubstituted C₁-C₆ alkyl, alkenyl, alkynyl, aryl, or mixtures thereof; and
R² is a substituted or unsubstituted C₁-C₆ alkyl, alkenyl, alkynyl, aryl, or mixtures thereof.

In an embodiment of Formula IIa, Y is S⁻. In an embodiment of Formula IIa, Y is O. In an embodiment of Formula IIa, X is OR¹ or F.

In an embodiment of Formula IIa, the base moiety B is selected from the group consisting of adenine, guanine, cytosine, and uracil.

In another aspect, the disclosure provides a crRNA and/or a tracrRNA comprising at least one modified intersubunit linkage of Formula II:

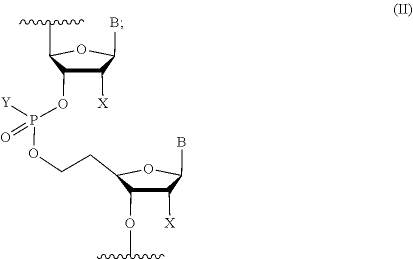

wherein:
B is a base moiety;
X is selected from the group consisting of H, OH, OR¹, R¹, F, Cl, Br, I, SH, SR¹, NH₂, NHR¹, NR¹₂, and COOR¹;
Y is selected from the group consisting of O⁻, OH, OR², NH⁻, NH₂, NR²₂, BH₃, S⁻, R², and SH;
Z is O or O(CH₂)ₙ² wherein n² is 1 to 10;
R¹ is a substituted or unsubstituted C₁-C₆ alkyl, alkenyl, alkynyl, aryl, or mixtures thereof;
R² is a substituted or unsubstituted C₁-C₆ alkyl, alkenyl, alkynyl, aryl, or mixtures thereof.

In an embodiment of Formula II, Y is S⁻. In an embodiment of Formula II, Y is O. In an embodiment of Formula II, X is OR¹ or F.

In an embodiment of Formula II, the base moiety B is selected from the group consisting of adenine, guanine, cytosine, and uracil.

In one aspect, the disclosure provides a crRNA and/or a tracrRNA comprising at least one modified intersubunit linkage of Formula IIIa:

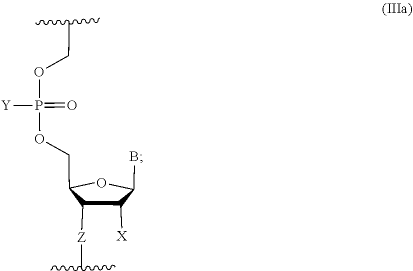

wherein:
B is a base moiety;
X is selected from the group consisting of H, OH, OR¹, R¹, F, Cl, Br, I, SH, SR¹, NH₂, NHR¹, NR¹₂, and COOR¹;
Y is selected from the group consisting of O⁻, OH, OR², NH⁻, NH₂, NR²₂, BH₃, S⁻, R², and SH;
Z is O or O(CH₂)ₙ² wherein n² is 1 to 10;
R¹ is a substituted or unsubstituted C₁-C₆ alkyl, alkenyl, alkynyl, aryl, or mixtures thereof; and
R² is a substituted or unsubstituted C₁-C₆ alkyl, alkenyl, alkynyl, aryl, or mixtures thereof.

In an embodiment of Formula IIIa, Y is S⁻. In an embodiment of Formula IIIa, X is OR¹ or F.

In an embodiment of Formula IIIa, the base moiety B is selected from the group consisting of adenine, guanine, cytosine, and uracil.

In another aspect, the disclosure provides a crRNA and/or a tracrRNA comprising at least one modified intersubunit linkage of Formula III:

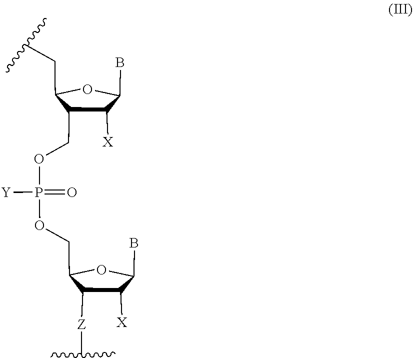

wherein:
B is a base moiety;
X is selected from the group consisting of H, OH, OR¹, R¹, F, Cl, Br, I, SH, SR¹, NH₂, NHR¹, NR¹₂, and COOR¹;
Y is selected from the group consisting of O⁻, OH, OR², NH⁻, NH₂, NR²₂, BH₃, S⁻, R², and SH;
Z is O or O(CH₂)ₙ² wherein n² is 1 to 10;
R¹ is a substituted or unsubstituted C₁-C₆ alkyl, alkenyl, alkynyl, aryl, or mixtures thereof; and
R² is a substituted or unsubstituted C₁-C₆ alkyl, alkenyl, alkynyl, aryl, or mixtures thereof

In an embodiment of Formula III, Y is S⁻. In an embodiment of Formula III, X is OR¹ or F.

In an embodiment of Formula III, the base moiety B is selected from the group consisting of adenine, guanine, cytosine, and uracil.

In one aspect, the disclosure provides a crRNA and/or a tracrRNA comprising at least one modified intersubunit linkage of Formula IVa:

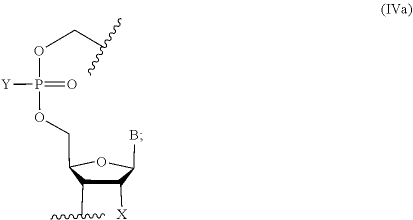

wherein:
B is a base moiety;
X is selected from the group consisting of H, OH, OR¹, R¹, F, Cl, Br, I, SH, SR¹, NH₂, NHR¹, NR¹₂, and COOR¹;
Y is selected from the group consisting of O⁻, OH, OR², NH⁻, NH₂, NR²₂, BH₃, S⁻, R², and SH;
Z is O or O(CH₂)ₙ² wherein n² is 1 to 10;
R¹ is a substituted or unsubstituted C₁-C₆ alkyl, alkenyl, alkynyl, aryl, or mixtures thereof; and
R² is a substituted or unsubstituted C₁-C₆ alkyl, alkenyl, alkynyl, aryl, or mixtures thereof.

In an embodiment of Formula IVa, Y is S⁻. In an embodiment of Formula IVa, X is OR¹ or F.

In an embodiment of Formula IVa, the base moiety B is selected from the group consisting of adenine, guanine, cytosine, and uracil.

In another aspect, the disclosure provides a crRNA and/or a tracrRNA comprising at least one modified intersubunit linkage of Formula IV:

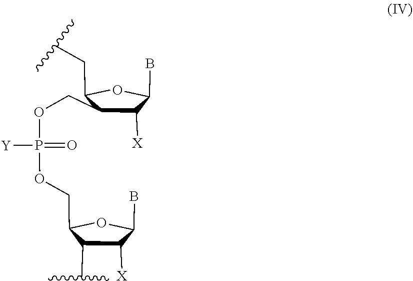

wherein:
B is a base moiety;
X is selected from the group consisting of H, OH, OR¹, R¹, F, Cl, Br, I, SH, SR¹, NH₂, NHR¹, NR¹₂, and COOR¹;
Y is selected from the group consisting of O⁻, OH, OR², NH⁻, NH₂, NR²₂, BH₃, S⁻, R², and SH;
R¹ is a substituted or unsubstituted C₁-C₆ alkyl, alkenyl, alkynyl, aryl, or mixtures thereof; and
R² is a substituted or unsubstituted C₁-C₆ alkyl, alkenyl, alkynyl, aryl, or mixtures thereof.

In an embodiment of Formula IV, Y is S⁻. In an embodiment of Formula IV, X is OR¹ or F.

In an embodiment of Formula IV, the base moiety B is selected from the group consisting of adenine, guanine, cytosine, and uracil.

In one aspect, the disclosure provides a crRNA and/or a tracrRNA comprising at least one modified intersubunit linkage of Formula Va:

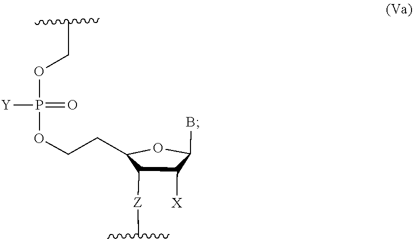

wherein:
B is a base moiety;
X is selected from the group consisting of H, OH, OR¹, R¹, F, Cl, Br, I, SH, SR¹, NH₂, NHR¹, NR¹₂, and COOR¹;
Y is selected from the group consisting of O⁻, OH, OR², NH⁻, NH₂, NR²₂, BH₃, S⁻, R², and SH;
Z is O or O(CH₂)ₙ² wherein n² is 1 to 10;
R¹ is a substituted or unsubstituted C₁-C₆ alkyl, alkenyl, alkynyl, aryl, or mixtures thereof; and
R² is a substituted or unsubstituted C₁-C₆ alkyl, alkenyl, alkynyl, aryl, or mixtures thereof.

In an embodiment of Formula Va, Y is S⁻. In an embodiment of Formula Va, X is OR¹ or F.

In an embodiment of Formula Va, the base moiety B is selected from the group consisting of adenine, guanine, cytosine, and uracil.

In another aspect, the disclosure provides a crRNA and/or a tracrRNA comprising at least one modified intersubunit linkage of Formula V:

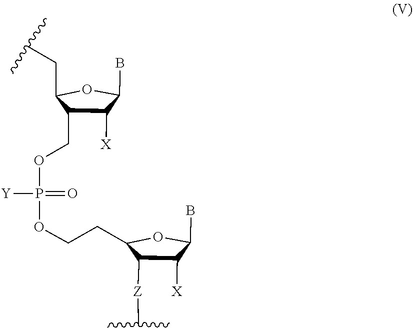

wherein:
B is a base moiety;
X is selected from the group consisting of H, OH, OR¹, R¹, F, Cl, Br, I, SH, SR¹, NH₂, NHR¹, NR¹₂, and COOR¹;
Y is selected from the group consisting of O⁻, OH, OR², NH⁻, NH₂, NR²₂, BH₃, S⁻, R², and SH;
Z is O or O(CH₂)ₙ² wherein n² is 1 to 10;
R¹ is a substituted or unsubstituted C₁-C₆ alkyl, alkenyl, alkynyl, aryl, or mixtures thereof; and
R² is a substituted or unsubstituted C₁-C₆ alkyl, alkenyl, alkynyl, aryl, or mixtures thereof.

In an embodiment of Formula V, Y is S⁻. In an embodiment of Formula V, X is OR¹ or F.

In an embodiment of Formula V, the base moiety B is selected from the group consisting of adenine, guanine, cytosine, and uracil.

In one aspect, the disclosure provides a crRNA and/or a tracrRNA comprising at least one modified intersubunit linkage of Formula VIa:

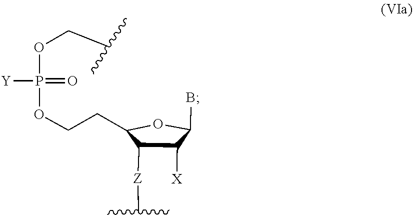

wherein:
B is a base moiety;
X is selected from the group consisting of H, OH, OR¹, R¹, F, Cl, Br, I, SH, SR¹, NH₂, NHR¹, NR¹₂, and COOR¹;
Y is selected from the group consisting of O⁻, OH, OR², NH⁻, NH₂, NR²₂, BH₃, S⁻, R², and SH;
Z is O or O(CH₂)ₙ² wherein n² is 1 to 10;
R¹ is a substituted or unsubstituted C₁-C₆ alkyl, alkenyl, alkynyl, aryl, or mixtures thereof; and
R² is a substituted or unsubstituted C₁-C₆ alkyl, alkenyl, alkynyl, aryl, or mixtures thereof.

In an embodiment of Formula VIa, Y is S⁻. In an embodiment of Formula VIa, X is OR¹ or F.

In an embodiment of Formula VIa, the base moiety B is selected from the group consisting of adenine, guanine, cytosine, and uracil.

In another aspect, the disclosure a crRNA and/or a tracrRNA comprising at least one modified intersubunit linkage of Formula VI:

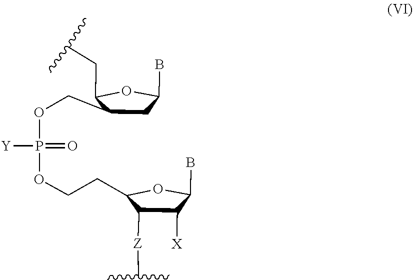

wherein:
B is a base moiety;
X is selected from the group consisting of H, OH, OR¹, R¹, F, Cl, Br, I, SH, SR¹, NH₂, NHR¹, NR¹₂, and COOR¹;
Y is selected from the group consisting of O⁻, OH, OR², NH⁻, NH₂, NR²₂, BH₃, S⁻, R², and SH;
Z is O or O(CH₂)ₙ² wherein n² is 1 to 10;
R¹ is a substituted or unsubstituted C₁-C₆ alkyl, alkenyl, alkynyl, aryl, or mixtures thereof; and
R² is a substituted or unsubstituted C₁-C₆ alkyl, alkenyl, alkynyl, aryl, or mixtures thereof.

In an embodiment of Formula VI, Y is S⁻. In an embodiment of Formula VI, X is OR¹ or F.

In an embodiment of Formula VI, the base moiety B is selected from the group consisting of adenine, guanine, cytosine, and uracil.

In certain embodiments, the crRNA portion of the disclosure comprises at least one (e.g., 1, 2, 3, 4, 5, 6, 7, 8, 9, 10, or more) exNA internucleotide linkage of Formula Ia. In certain embodiments, the crRNA portion of the disclosure comprises at least one (e.g., 1, 2, 3, 4, 5, 6, 7, 8, 9, 10, or more) exNA internucleotide linkage of Formula I. In certain embodiments, the crRNA portion of the disclosure comprises at least one (e.g., 1, 2, 3, 4, 5, 6, 7, 8, 9, 10, or more) exNA internucleotide linkage of Formula IIa. In certain embodiments, the crRNA portion of the disclosure comprises at least one (e.g., 1, 2, 3, 4, 5, 6, 7, 8, 9, 10, or more) exNA internucleotide linkage of Formula II. In certain embodiments, the crRNA portion of the disclosure comprises at least one (e.g., 1, 2, 3, 4, 5, 6, 7, 8, 9, 10, or more) exNA internucleotide linkage of Formula IIIa. In certain embodiments, the crRNA portion of the disclosure comprises at least one (e.g., 1, 2, 3, 4, 5, 6, 7, 8, 9, 10, or more) exNA internucleotide linkage of Formula III. In certain embodiments, the crRNA portion of the disclosure comprises at least one (e.g., 1, 2, 3, 4, 5, 6, 7, 8, 9, 10, or more) exNA internucleotide linkage of Formula IVa. In certain embodiments, the crRNA portion of the disclosure comprises at least one (e.g., 1, 2, 3, 4, 5, 6, 7, 8, 9, 10, or more) exNA internucleotide linkage of Formula IV. In certain embodiments, the crRNA portion of the disclosure comprises at least one (e.g., 1, 2, 3, 4, 5, 6, 7, 8, 9, 10, or more) exNA internucleotide linkage of Formula Va. In certain embodiments, the crRNA portion of the disclosure comprises at least one (e.g., 1, 2, 3, 4, 5, 6, 7, 8, 9, 10, or more) exNA internucleotide linkage of Formula V. In certain embodiments, the crRNA portion of the disclosure comprises at least one (e.g., 1, 2, 3, 4, 5, 6, 7, 8, 9, 10, or more) exNA internucleotide linkage of Formula VIa. In certain embodiments, the crRNA portion of the disclosure comprises at least one (e.g., 1, 2, 3, 4, 5, 6, 7, 8, 9, 10, or more) exNA internucleotide linkage of Formula VI.

In certain embodiments, the tracrRNA portion of the disclosure comprises at least one (e.g., 1, 2, 3, 4, 5, 6, 7, 8, 9, 10, or more) exNA internucleotide linkage of Formula Ia. In certain embodiments, the tracrRNA portion of the disclosure comprises at least one (e.g., 1, 2, 3, 4, 5, 6, 7, 8, 9, 10, or more) exNA internucleotide linkage of Formula I. In certain embodiments, the tracrRNA portion of the disclosure comprises at least one (e.g., 1, 2, 3, 4, 5, 6, 7, 8, 9, 10, or more) exNA internucleotide linkage of Formula IIa. In certain embodiments, the tracrRNA portion of the disclosure comprises at least one (e.g., 1, 2, 3, 4, 5, 6, 7, 8, 9, 10, or more) exNA internucleotide linkage of Formula II. In certain embodiments, the tracrRNA portion of the disclosure comprises at least one (e.g., 1, 2, 3, 4, 5, 6, 7, 8, 9, 10, or more) exNA internucleotide linkage of Formula IIIa. In certain embodiments, the tracrRNA portion of the disclosure comprises at least one (e.g., 1, 2, 3, 4, 5, 6, 7, 8, 9, 10, or more) exNA internucleotide linkage of Formula III. In certain embodiments, the tracrRNA portion of the disclosure comprises at least one (e.g., 1, 2, 3, 4, 5, 6, 7, 8, 9, 10, or more) exNA internucleotide linkage of Formula IVa. In certain embodiments, the tracrRNA portion of the disclosure comprises at least one (e.g., 1, 2, 3, 4, 5, 6, 7, 8, 9, 10, or more) exNA internucleotide linkage of Formula IV. In certain embodiments, the tracrRNA portion of the disclosure comprises at least one (e.g., 1, 2, 3, 4, 5, 6, 7, 8, 9, 10, or more) exNA internucleotide linkage of Formula Va. In certain embodiments, the tracrRNA portion of the disclosure comprises at least one (e.g., 1, 2, 3, 4, 5, 6, 7, 8, 9, 10, or more) exNA internucleotide linkage of Formula V. In certain embodiments, the tracrRNA portion of the disclosure comprises at least one (e.g., 1, 2, 3, 4, 5, 6, 7, 8, 9, 10, or more) exNA internucleotide linkage of Formula VIa. In certain embodiments, the tracrRNA portion of the disclosure comprises at least one (e.g., 1, 2, 3, 4, 5, 6, 7, 8, 9, 10, or more) exNA internucleotide linkage of Formula VI.

Exemplary chemical modification patterns for exNA-containing crRNA and tracrRNA sequences are described in Table 3 and Table 4 below. Any of the exNA-containing crRNAs of Table 3 can be paired with any of the exNA-containing tracrRNAs of Table 4 or any of the non-exNA-containing tracrRNAs of Table 2. Similarly, any of the exNA-containing tracrRNAs of Table 4 can be paired with any of the exNA-containing crRNAs of Table 3 or any of the non-exNA-containing crRNAs of Table 1.

TABLE 3
Exemplary chemical modification patterns for
exNA-containing crRNAs
KEY: rN = RNA, mN = 2′-O-methyl RNA, fN =
2′-fluoro RNA, NexN = exNA linkage, N = A,
U, G, or C N#N = phosphorothioate linkage

| Name | |
|---|---|
| (Alt. | |
| Name) | Sequence |
| crRNA | mN#mN#mN#mNmNmNmNmNmNmNfNfNfNfNrN#rN#f |
| Ex20- | NfNrN#mNmGrU#rU#rU#fUfAmGmAmGmCmUmAmU# |
| 1 | mG#mC#exmU |
| (36C- | (SEQ ID NO: 5) |
| ExU) | |
| crRNA | mN#mN#mN#mNmNmNmNmNmNmNfNfNfNfNrN#rN#f |
| Ex20- | NfNrN#mNmGrU#rU#rU#fUfAmGmAmGmCmUmAexmU |
| 4 | #mG#mC#mU |
| (33C- | (SEQ ID NO: 6) |
| ExU) | |
| crRNA | mN#mN#mN#mNmNmNmNmNmNmNfNfNfNfNrN#rN#f |
| Ex20- | NfNrN#mNmGrU#rU#rU#fUfAmGmAmGmCexmUmAmU |
| 5 | #mG#mC#mU |
| (31C- | (SEQ ID NO: 7) |
| ExU) | |
| crRNA | mN#mN#mN#mNmNmNmNmNmNmNfNfNfNfNrN#rN#f |
| Ex20- | NfNrN#mNmGrU#rU#rU#exfUfAmGmAmGmCmUmAmU |
| 7 | #mG#mC#mU |
| (25C- | (SEQ ID NO: 8) |
| ExU) | |
| crRNA | mN#mN#mN#mNmNmNmNmNmNmNfNfNfNfNrN#rN#f |
| Ex20- | NfNrN#mNmGrU#rU#exrU#fUfAmGmAmGmCmUmAmU |
| 8 | #mG#mC#mU |
| (24C- | (SEQ ID NO: 9) |
| ExU) | |
| crRNA | mN#mN#mN#mNmNmNmNmNmNmNfNfNfNfNrN#rN#f |
| Ex20- | NfNrN#mNmGrU#exrU#rU#fUfAmGmAmGmCmUmAmU |
| 9 | #mG#mC#mU |
| (23C- | (SEQ ID NO: 10) |
| ExU) | |
| crRNA | mN#mN#mN#mNmNmNmNmNmNmNfNfNfNfNrN#rN#f |
| Ex20- | NfNrN#mNmGexrU#rU#rU#fUfAmGmAmGmCmUmAmU |
| 10 | #mG#mC#mU |
| (22C- | (SEQ ID NO: 11) |
| ExU) | |
| crRNA | mN#mN#mN#mNmNmNmNmNmNmNfNfNfNfNrN#rN#f |
| Ex20- | NexfNrN#mNmGrU#rU#rU#fUfAmGmAmGmCmUmAmU |
| 12 | #mG#mC#mU |
| (18C- | (SEQ ID NO: 12) |
| ExU) | |
| crRNA | mN#mN#mN#mNmNmNmNmNmNmNfNfNfNexfNrN#rN# |
| Ex20- | fNfNrN#mNmGrU#rU#rU#fUfAmGmAmGmCmUmAmU# |
| 13 | mG#mC#Mu |
| (14C- | (SEQ ID NO: 13) |
| ExU) | |
| crRNA | mN#mN#mN#mNmNmNmNmNexmNmNfNfNfNfNrN#rN# |
| Ex20- | fNfNrN#mNmGrU#rU#rU#fUfAmGmAmGmCmUmAmU# |
| 15 | mG#mC#mU |
| (9C- | (SEQ ID NO: 14) |
| ExU) | |

TABLE 4
Exemplary chemical modification patterns for
exNA-containing tracrRNAs
KEY: rN = RNA, mN = 2′-O-methyl RNA, fN =
2′-fluoro RNA, NexN = exNA linkage, N = A, U,
G, or C N#N = phosphorothioate linkage

| Name | |
|---|---|
| (Alt. | |
| Name) | Sequence |
| tracrRNA | mA#mG#mC#mAmUmAmGmCmAmAmGrUrUmAr |
| 2 | AmAmArUmAmAmGmGrCrUmArGrUrCmCrGr |
| | UrUmAmUmCmAmAmCmUmUmGmAmAmAmAmAm |
| | GmUmGmGmCmAmCmCmGmAmGmUmCmGmGmUm |
| | GmC#mU#mU#mU |
| | (SEQ ID NO: 15) |
| tracrRNA | mA#mG#mC#mAmUmAmGmCmAmAmGrUrUmAr |
| 2-1 | AmAmArUmAmAmGmGrCrUmArGrUrCmCrGr |
| (67T- | UrUmAmUmCmAmAmCmUmUmGmAmAmAmAmAm |
| ExU) | GmUmGmGmCmAmCmCmGmAmGmUmCmGmGmUm |
| | GmC#mU#mU#exmU |
| | (SEQ ID NO: 16) |
| tracrRNA | mA#mG#mC#mAmUmAmGmCmAmAmGrUrUmAr |
| 2-2 | AmAmArUmAmAmGmGrCrUmArGrUrCmCrGr |
| (66T- | UrUmAmUmCmAmAmCmUmUmGmAmAmAmAmAm |
| ExU) | GmUmGmGmCmAmCmCmGmAmGmUmCmGmGmUm |
| | GmC#mU#exmU#mU |
| | (SEQ ID NO: 17) |
| tracrRNA | mA#mG#mC#mAmUmAmGmCmAmAmGrUrUmAr |
| 2-3 | AmAmArUmAmAmGmGrCrUmArGrUrCmCrGr |
| (65T- | UrUmAmUmCmAmAmCmUmUmGmAmAmAmAmAm |
| ExU) | GmUmGmGmCmAmCmCmGmAmGmUmCmGmGmUm |
| | GmC#exmU#mU#mU |
| | (SEQ ID NO: 18) |
| tracrRNA | mA#mG#mC#mAmUmAmGmCmAmAmGrUrUmAr |
| 2-5 | AmAmArUmAmAmGmGrCrUmArGrUrCmCrGr |
| (66/67T- | UrUmAmUmCmAmAmCmUmUmGmAmAmAmAmAm |
| ExU) | GmUmGmGmCmAmCmCmGmAmGmUmCmGmGmUm |
| | GmC#mU#exmU#exmU |
| | (SEQ ID NO: 19) |
| tracrRNA | mA#mG#mC#mAmUmAmGmCmAmAmGrUrUmAr |
| 2-6 | AmAmArUmAmAmGmGrCrUmArGrUrCmCrGr |
| (65/66/ | UrUmAmUmCmAmAmCmUmUmGmAmAmAmAmAm |
| 67T-ExU) | GmUmGmGmCmAmCmCmGmAmGmUmCmGmGmUm |
| | GmC#exmU#exmU#exmU |
| | (SEQ ID NO: 20) |
| tracrRNA | mA#mG#mC#mAmUmAmGmCmAmAmGrUrUmAr |
| 2-8 | AmAmArUmAmAmGmGrCrUmArGrUrCmCrGr |
| (62T- | UrUmAmUmCmAmAmCmUmUmGmAmAmAmAmAm |
| ExU) | GmUmGmGmCmAmCmCmGmAmGmUmCmGmGexm |
| | UmGmC#mU#mU#mU |
| | (SEQ ID NO: 21) |
| tracrRNA | mA#mG#mC#mAmUmAmGmCmAmAmGrUrUmAr |
| 2-10 | AmAmArUmAmAmGmGrCrUmArGrUrCmCrGr |
| (58T- | UrUmAmUmCmAmAmCmUmUmGmAmAmAmAmAm |
| ExU) | GmUmGmGmCmAmCmCmGmAmGexmUmCmGmGm |
| | UmGmC#mU#mU#mU |
| | (SEQ ID NO: 22) |
| tracrRNA | mA#mG#mC#mAmUmAmGmCmAmAmGrUrUmAr |
| 2-14 | AmAmArUmAmAmGmGrCrUmArGrUrCmCrGr |
| (48T- | UrUmAmUmCmAmAmCmUmUmGmAmAmAmAmAm |
| ExU) | GexmUmGmGmCmAmCmCmGmAmGmUmCmGmGm |
| | UmGmC#mU#mU#mU |
| | (SEQ ID NO: 23) |
| tracrRNA | mA#mG#mC#mAmUmAmGmCmAmAmGrUrUmAr |
| 2-15 | AmAmArUmAmAmGmGrCrUmArGrUrCmCrGr |
| (40T- | UrUmAmUmCmAmAmCmUexmUmGmAmAmAmAm |
| ExU) | AmGmUmGmGmCmAmCmCmGmAmGmUmCmGmGm |
| | UmGmC#mU#mU#mU |
| | (SEQ ID NO: 24) |
| tracrRNA | mA#mG#mC#mAmUmAmGmCmAmAmGrUrUmAr |
| 2-16 | AmAmArUmAmAmGmGrCrUmArGrUrCmCrGr |
| (39T- | UrUmAmUmCmAmAmCexmUmUmGmAmAmAmAm |
| ExU) | AmGmUmGmGmCmAmCmCmGmAmGmUmCmGmGm |
| | UmGmC#mU#mU#mU |
| | (SEQ ID NO: 25) |
| tracrRNA | mA#mG#mC#mAmUmAmGmCmAmAmGrUrUmAr |
| 2-19 | AmAmArUmAmAmGmGrCrUmArGrUrCmCrGr |
| (34T- | UrUmAexmUmCmAmAmCmUmUmGmAmAmAmAm |
| ExU) | AmGmUmGmGmCmAmCmCmGmAmGmUmCmGmGm |
| | UmGmC#mU#mU#mU |
| | (SEQ ID NO: 26) |
| tracrRNA | mA#mG#mC#mAmUmAmGmCmAmAmGrUrUmAr |
| 2-20 | AmAmArUmAmAmGmGrCrUmArGrUrCmCrGr |
| (32T- | UexrUmAmUmCmAmAmCmUmUmGmAmAmAmAm |
| ExU) | AmGmUmGmGmCmAmCmCmGmAmGmUmCmGmGm |
| | UmGmC#mU#mU#mU |
| | (SEQ ID NO: 27) |
| tracrRNA | mA#mG#mC#mAmUmAmGmCmAmAmGrUrUmAr |
| 2-21 | AmAmArUmAmAmGmGrCrUmArGrUrCmCrGe |
| (31T- | xrUrUmAmUmCmAmAmCmUmUmGmAmAmAmAm |
| ExU) | AmGmUmGmGmCmAmCmCmGmAmGmUmCmGmGm |
| | UmGmC#mU#mU#mU |
| | (SEQ ID NO: 28) |
| tracrRNA | mA#mG#mC#mAmUmAmGmCmAmAmGrUrUmAr |
| 2-23 | AmAmArUmAmAmGmGrCrUmArGexrUrCmCr |
| (27T- | GrUrUmAmUmCmAmAmCmUmUmGmAmAmAmAm |
| ExU) | AmGmUmGmGmCmAmCmCmGmAmGmUmCmGmGm |
| | UmGmC#mU#mU#mU |
| | (SEQ ID NO: 29) |
| tracrRNA | mA#mG#mC#mAmUmAmGmCmAmAmGrUrUmAr |
| 2-24 | AmAmArUmAmAmGmGrCexrUmArGrUrCmCr |
| (24T- | GrUrUmAmUmCmAmAmCmUmUmGmAmAmAmAm |
| ExU) | AmGmUmGmGmCmAmCmCmGmAmGmUmCmGmGm |
| | UmGmC#mU#mU#mU |
| | (SEQ ID NO: 30) |
| tracrRNA | mA#mG#mC#mAmUmAmGmCmAmAmGrUrUmAr |
| 2-25 | AmAmAexrUmAmAmGmGrCrUmArGrUrCmCr |
| (18T- | GrUrUmAmUmCmAmAmCmUmUmGmAmAmAmAm |
| ExU) | AmGmUmGmGmCmAmCmCmGmAmGmUmCmGmGm |
| | UmGmC#mU#mU#mU |
| | (SEQ ID NO: 31) |
| tracrRNA | mA#mG#mC#mAmUmAmGmCmAmAmGrUexrUm |
| 2-26 | ArAmAmArUmAmAmGmGrCrUmArGrUrCmCr |
| (13T-ExU) | GrUrUmAmUmCmAmAmCmUmUmGmAmAmAmAm |
| | AmGmUmGmGmCmAmCmCmGmAmGmUmCmGmGm |
| | UmGmC#mU#mU#mU |
| | (SEQ ID NO: 32) |
| tracrRNA | mA#mG#mC#mAmUmAmGmCmAmAmGexrUrUm |
| 2-27 | ArAmAmArUmAmAmGmGrCrUmArGrUrCmCr |
| (12T- | GrUrUmAmUmCmAmAmCmUmUmGmAmAmAmAm |
| ExU) | AmGmUmGmGmCmAmCmCmGmAmGmUmCmGmGm |
| | UmGmC#mU#mU#mU |
| | (SEQ ID NO: 33) |
| tracrRNA | mA#mG#mC#mAexmUmAmGmCmAmAmGrUrUm |
| 2-30 | ArAmAmArUmAmAmGmGrCrUmArGrUrCmCr |
| (5T- | GrUrUmAmUmCmAmAmCmUmUmGmAmAmAmAm |
| ExU) | AmGmUmGmGmCmAmCmCmGmAmGmUmCmGmGm |
| | UmGmC#mU#mU#mU |
| | (SEQ ID NO: 34) |
| tracrRNA | mA#mG#mC#mAmUmAmGmCmAmAmGexrUrUm |
| 2-31 | ArAmAmArUmAmAmGmGrCexrUmArGrUrCm |
| (12/24/ | CrGrUrUmAmUmCmAmAmCmUmUmGmAmAmAm |
| 3′T- | AmAmGmUmGmGmCmAmCmCmGmAmGmUmCmGm |
| ExU) | GmUmGmC#exmU#exmU#exmU |
| | (SEQ ID NO: 35) |
| tracrRNA | mA#mG#mC#mAmUmAmGmCmAmAmGrUrUmAr |
| 2-32 | AmAmArUmAmAmGmGrCrUmArGrUrCmCrGr |
| (M32T- | UexmUmAmUmCmAmAmCmUmUmGmAmAmAmAm |
| ExU) | AmGmUmGmGmCmAmCmCmGmAmGmUmCmGmGm |
| | UmGmC#mU#mU#mU |
| | (SEQ ID NO: 36) |
| tracrRNA | mA#mG#mC#mAmUmAmGmCmAmAmGrUrUmAr |
| 2-33 | AmAmArUmAmAmGmGrCrUmArGrUrCmCrGe |
| (M31T- | xmUrUmAmUmCmAmAmCmUmUmGmAmAmAmAm |
| ExU) | AmGmUmGmGmCmAmCmCmGmAmGmUmCmGmGm |
| | UmGmC#mU#mU#mU |
| | (SEQ ID NO: 37) |
| tracrRNA | mA#mG#mC#mAmUmAmGmCmAmAmGrUrUmAr |
| 2-34 | AmAmArUmAmAmGmGrCrUmArGexmUrCmCr |
| (M27T- | GrUrUmAmUmCmAmAmCmUmUmGmAmAmAmAm |
| ExU) | AmGmUmGmGmCmAmCmCmGmAmGmUmCmGmGm |
| | UmGmC#mU#mU#mU |
| | (SEQ ID NO: 38) |
| tracrRNA | mA#mG#mC#mAmUmAmGmCmAmAmGrUrUmAr |
| 2-35 | AmAmAexmUmAmAmGmGrCrUmArGrUrCmCr |
| (M18T- | GrUrUmAmUmCmAmAmCmUmUmGmAmAmAmAm |
| ExU) | AmGmUmGmGmCmAmCmCmGmAmGmUmCmGmGm |
| | UmGmC#mU#mU#mU |
| | (SEQ ID NO: 39) |
| tracrRNA | mA#mG#mC#mAmUmAmGmCmAmAmGrUexmUm |
| 2-36 | ArAmAmArUmAmAmGmGrCrUmArGrUrCmCr |
| (M13T- | GrUrUmAmUmCmAmAmCmUmUmGmAmAmAmAm |
| ExU) | AmGmUmGmGmCmAmCmCmGmAmGmUmCmGmGm |
| | UmGmC#mU#mU#mU |
| | (SEQ ID NO: 40) |
| tracrRNA | mA#mG#mC#mAmUmAmGmCmAmAmGexmUrUm |
| 102 | ArAmAmAexmUmAmAmGmGrCrUmArGrUrCm |
| (M12/ | CrGrUrUmAmUmCmAmAmCmUmUmGmAmAmAm |
| M18/ | AmAmGmUmGmGmCmAmCmCmGmAmGmUmCmGm |
| 3′T- | GmUmGmC#exmU#exmU#exmU |
| ExU) | (SEQ ID NO: 41) |
| tracrRNA | mA#mG#mC#mAmUmAmGmCmAmAmGexmUrUm |
| 103 | AfAmAmAexmUmAmAmGmGfCrUmAfGrUfCm |
| (6F- | CfGrUfUmAmUmCmAmAmCmUmUmGmAmAmAm |
| M12/ | AmAmGmUmGmGmCmAmCmCmGmAmGmUmCmGm |
| M18/ | GmUmGmC#exmU#exmU#exmU |
| 3′T- | (SEQ ID NO: 42) |
| ExU) | |

It will be understood to those of skill in the art that the base sequence of the first 20 nucleotides of the exemplary crRNAs recited in Table 1 or Table 3 above are directed to a specific target. This 20-nucleotide base sequence may be changed based on the target nucleic acid, however the chemical modifications remain the same. An exemplary unmodified crRNA sequence, from 5′ to 3′, is NNNNNNNNNNNNNNNNNNNNGUUUUAGAGCUAUGCU (SEQ ID NO: 1), where “N” corresponds to any nucleotide (e.g., A, U, G, or C). An exemplary unmodified tracrRNA sequence, from 5′ to 3′, is AGCAUAGCAAGUUAAAAUAAGGCUAGUCCGUUAUCAACUUGAAAAAGU GGCACCGAGUCGGUGCUUU (SEQ ID NO: 2).

It will be further understood to those of skill in the art that the guide sequence may be 10-30 nucleotides in length, preferably 16-24 nucleotides in length (for example, 16, 17, 18, 19, 20, 21, 22, 23 or 24 nucleotides in length), and is at or near the 5′ terminus of a Cas9 gRNA.

### High-Affinity Repeat/Anti-Repeat Guide RNA Modifications

A crRNA and a tracrRNA hybridize together by forming a duplex between the repeat region of the crRNA and the anti-repeat region of the tracrRNA (see FIG. 1). In certain embodiments, modular, or dual RNA, guide RNAs are provided with modifications in the repeat region and the anti-repeat region to enhance the affinity between the two regions and form a stronger duplex.

The high-affinity interaction may be enhanced by increasing the GC nucleotide content in the duplex formed by the repeat regions and the anti-repeat region. Nucleotide modifications, such as 2′-Fluoro and 2′-O-Methyl modifications, may also be introduced, which increase the melting temperature (Tm) of the duplex. Further modifications include the use of orthogonal and non-naturally occurring nucleotides.

The various repeat region/anti-repeat region modifications described herein enhance the stability of the duplex, helping to prevent the crRNA and tracrRNA from folding into sub-optimal structures and therefore promoting higher genome editing efficacy.

The use of a modular, or dual RNA, guide RNA approach over a single guide RNA (sgRNA) approach has several advantages, including the ease of making the shorter crRNA and tracrRNA relative to a longer sgRNA, and the reduced cost of manufacturing the dual RNAs relative to the sgRNA. Exemplary crRNAs and tracrRNAs with modifications in the repeat and anti-repeat region, including a high GC content and 2′-Fluoro modifications, are shown in Table 5 and Table 6 below.

TABLE 5
Exemplary modified repeat crRNAs.
KEY: rN = RNA, mN = 2′-O-methyl RNA,
fN = 2′-fluoro RNA, N = A, U, G, or C
N#N = phosphorothioate linkage

| Name | Sequence |
|---|---|
| hiGC | NNNNNNNNNNNNNNNNNNNNGUUUUAGAGCGA |
| repeat | GCGC |
| crRNA | (SEQ ID NO: 3) |
| hiGC & | NNNNNNNNNNNNNNNNNNNNmGrU#rU#rU#fUfAm |
| 2′- | GmAfGfCfGfAfG#fC#mG#mC |
| fluoro | (SEQ ID NO: 281) |
| repeat | |
| crRNA | |

It will be understood that the hiGC repeat crRNA above may further comprise any of the crRNA chemical modification patterns as recited in Table 1 and

Table 3 above.

TABLE 6
Exemplary modified repeat tracrRNAs
KEY: rN = RNA, mN = 2′-O-methyl RNA,
fN = 2′-fluoro RNA, N = A, U, G, or C
N#N = phosphorothioate linkage

| Name | Sequence |
|---|---|
| hiGC | GCGCUCGCAAGUUAAAAUAAGGCUAGUCCGU |
| anti- | UAUCAACUUGAAAAAGUGGCACCGAGUCGGU |
| repeat | GCUUU |
| tracrRNA | (SEQ ID NO: 4) |
| hiGC & | mG#mC#fG#fCfUfCfGfCmAmAmGrUrUmArAmAm |
| 2′-fluoro | ArUmAmAmGmGrCrUmArGrUrCmCrGrUrUmAmU |
| anti- | mCmAmAmCmUmUmGmAmAmAmAmAmGmUmG |
| repeat | mGmCmAmCmCmGmAmGmUmCmGmGmUmGmC |
| tracrRNA | #mU#mU#Mu |
| | (SEQ ID NO: 282) |

It will be understood that the hiGC anti-repeat tracrRNA above may further comprise any of the tracrRNA chemical modification patterns, as recited in Table 2 and Table 4 above.

### Guide RNA Conjugates

The chemically modified guide RNAs of the disclosure may be modified with terminally conjugated moieties. As used herein, a “terminally conjugated moiety” or “moiety” refers to a compound which may be linked or attached to the 5′ and/or 3′ end of the crRNA and/or tracrRNA of a guide RNA. Terminally conjugated moieties can provide increased stability, increased ability to penetrate cell membranes, increase cellular uptake, increase circulation time in vivo, act as a cell-specific directing reagent, and/or provide a means to monitor cellular or tissue-specific uptake.

In certain embodiments, the terminally conjugated moiety is conjugated to the 5′ end of the crRNA portion of a guide RNA. In certain embodiments, the terminally conjugated moiety is conjugated to the 3′ end of the crRNA portion of a guide RNA. In certain embodiments, the terminally conjugated moiety is conjugated to the 5′ end of the tracrRNA portion of a guide RNA. In certain embodiments, the terminally conjugated moiety is conjugated to the 3′ end of the tracrRNA portion of a guide RNA.

In certain exemplary embodiments, a terminally conjugated moiety includes, but is not limited to, fatty acid, steroid, secosteroid, lipid, ganglioside analog, nucleoside analogs, endocannabinoid, vitamin, receptor ligand, peptide, aptamer, alkyl chain, fluorophore, antibody, nuclear localization signal, and the like.

In certain exemplary embodiments, a terminally conjugated moiety includes, but is not limited to, cholesterol, cholesterol-triethylene glycol (TEGChol), docosahexaenoic acid (DHA), docosanoic acid (DCA), lithocholic acid (LA), GalNAc, amphiphilic block copolymer (ABC), hydrophilic block copolymer (HBC), poloxamer, Cy5, Cy3, and the like.

In certain exemplary embodiments, the at least one terminally conjugated moiety is a modified lipid, including a branched lipid (such as the structure shown in Formula I) or a headgroup-modified lipid (such as the structure shown in Formula II).

X-MC(═Y)M-Z-[L-MC(═Y)M-R]n Formula I:

where X is a moiety that links the lipid to the guide RNA, each Y is independently oxygen or sulfur, each M is independently CH₂, NH, O or S, Z is a branching group which allows two or three (“n”) chains to be joined to the rest of the structure, L is an optional linker moiety, and each R is independently a saturated, monounsaturated or polyunsaturated linear or branched moiety from 2 to 30 atoms in length, a sterol, or other hydrophobic group.

X-MC(═Y)M-Z-[L-MC(═Y)M-R]n-L-K-J Formula II:

where X is a moiety that links the lipid to the guide RNA, each Y is independently oxygen or sulfur, each M is independently CH₂, NH, N-alkyl, O or S, Z is a branching group which allows two or three (“n”) chains to be joined to the rest of the structure, each L is independently an optional linker moiety, and R is a saturated, monounsaturated or polyunsaturated linear or branched moiety from 2 to 30 atoms in length, a sterol, or other hydrophobic group, K is a phosphate, sulfate, or amide and J is an aminoalkane or quaternary aminoalkane group.

The moieties may be attached to the terminal nucleotides of the guide RNA via a linker. Exemplary linkers include, but are not limited to, an ethylene glycol chain, an alkyl chain, a polypeptide, a polysaccharide, a block copolymer, and the like.

In certain embodiments, the moiety is conjugated to the 5′ end and/or 3′ end of any one of crRNA Ex20-1, crRNA Ex20-4, crRNA Ex20-5, crRNA Ex20-7, crRNA Ex20-8, crRNA Ex20-9, crRNA Ex20-10, crRNA Ex20-12, crRNA Ex20-13, or crRNA Ex20-15.

In certain embodiments, the moiety is conjugated to the 5′ end and/or 3′ end of any one of tracrRNA 2-1 (67T-ExU), tracrRNA 2-2 (66T-ExU), tracrRNA 2-3 (65T-ExU), tracrRNA 2-5 (66/67T-ExU), tracrRNA 2-6 (65/66/67T-ExU), tracrRNA 2-8 (62T-ExU), tracrRNA 2-10 (58T-ExU), tracrRNA 2-14 (48T-ExU), tracrRNA 2-15 (40T-ExU), tracrRNA 2-16 (39T-ExU), tracrRNA 2-19 (34T-ExU), tracrRNA 2-20 (32T-ExU), tracrRNA 2-21 (31T-ExU), tracrRNA 2-23 (27T-ExU), tracrRNA 2-24 (24T-ExU), tracrRNA 2-25 (18T-ExU), tracrRNA 2-26 (13T-ExU), tracrRNA 2-27 (12T-ExU), tracrRNA 2-30 (5T-ExU), tracrRNA 2-31 (12/24/3′T-ExU), tracrRNA 2-32 (M32T-ExU), tracrRNA 2-33 (M31T-ExU), tracrRNA 2-34 (M27T-ExU), tracrRNA 2-35 (M18T-ExU), tracrRNA 2-36 (M13T-ExU), tracrRNA 102 (M12/M18/3′T-ExU), or tracrRNA 103 (6F-M12/M18/3′T-ExU).

### Chemically Modified Single Guide RNA

As described herein, the chemically modified guide RNAs of the disclosure may be constructed as single guide RNAs (sgRNAs) by linking the 3′ end of a crRNA to the 5′ end of a tracrRNA. The linker may be an oligonucleotide loop, including a chemically modified oligonucleotide loop. In certain embodiments, the oligonucleotide loop comprises a GAAA tetraloop. The linker may be a non-nucleotide chemical linker, including, but not limited to, ethylene glycol oligomers (see, e.g., Pils et al. Nucleic Acids Res. 28(9): 1859-1863 (2000)).

### RNA-Guided Nucleases

RNA-guided nucleases according to the present disclosure include, without limitation, naturally-occurring Type II CRISPR nucleases such as Cas9, as well as other nucleases derived or obtained therefrom. Exemplary Cas9 nucleases that may be used in the present disclosure include, but are not limited to, S. pyogenes Cas9 (SpCas9), S. aureus Cas9 (SaCas9), N. meningitidis Cas9 (NmCas9), C. jejuni Cas9 (CjCas9), and Geobacillus Cas9 (GeoCas9). In functional terms, RNA-guided nucleases are defined as those nucleases that: (a) interact with (e.g., complex with) a gRNA: and (b) together with the gRNA, associate with, and optionally cleave or modify, a target region of a DNA that includes (i) a sequence complementary to the targeting domain of the gRNA and, optionally, (ii) an additional sequence referred to as a “protospacer adjacent motif,” or “PAM,” which is described in greater detail below. As the following examples will illustrate, RNA-guided nucleases can be defined, in broad terms, by their PAM specificity and cleavage activity, even though variations may exist between individual RNA-guided nucleases that share the same PAM specificity or cleavage activity. Skilled artisans will appreciate that some aspects of the present disclosure relate to systems, methods and compositions that can be implemented using any suitable RNA-guided nuclease having a certain PAM specificity and/or cleavage activity. For this reason, unless otherwise specified, the term RNA-guided nuclease should be understood as a generic term, and not limited to any particular type (e.g., Cas9 vs. Cpfl), species (e.g., S. pyogenes vs. S. aureus) or variation (e.g., full-length vs. truncated or split: naturally-occurring PAM specificity vs. engineered PAM specificity).

Various RNA-guided nucleases may require different sequential relationships between PAMs and protospacers. In general, Cas9s recognize PAM sequences that are 5′ of the protospacer as visualized relative to the top or complementary strand.

In addition to recognizing specific sequential orientations of PAMs and protospacers, RNA-guided nucleases generally recognize specific PAM sequences. S. aureus Cas9, for example, recognizes a PAM sequence of NNGRRT, wherein the N sequences are immediately 3′ of the region recognized by the gRNA targeting domain. S. pyogenes Cas9 recognizes NGG PAM sequences. It should also be noted that engineered RNA-guided nucleases can have PAM specificities that differ from the PAM specificities of similar nucleases (such as the naturally occurring variant from which an RNA-guided nuclease is derived, or the naturally occurring variant having the greatest amino acid sequence homology to an engineered RNA-guided nuclease). Modified Cas9s that recognize alternate PAM sequences are described below.

RNA-guided nucleases are also characterized by their DNA cleavage activity: naturally-occurring RNA-guided nucleases typically form DSBs in target nucleic acids, but engineered variants have been produced that generate only SSBs (discussed above: see also Ran 2013, incorporated by reference herein), or that do not cut at all.

The RNA-guided nuclease Cas9 may be a variant of Cas9 with altered activity. Exemplary variant Cas9 nucleases include, but are not limited to, a Cas9 nickase (nCas9), a catalytically dead Cas9 (dCas9), a hyper accurate Cas9 (HypaCas9) (Chen et al. Nature, 550(7676), 407-410 (2017)), a high fidelity Cas9 (Cas9-HF) (Kleinstiver et al. Nature 529(7587), 490-495 (2016)), an enhanced specificity Cas9 (eCas9) (Slaymaker et al. Science 351(6268), 84-88 (2016)), and an expanded PAM Cas9 (xCas9) (Hu et al. Nature doi: 10.1038/nature26155 (2018)).

The RNA-guided nucleases may be combined with the chemically modified guide RNAs of the present disclosure to form a genome-editing system. The RNA-guided nucleases may be combined with the chemically modified guide RNAs to form an RNP complex that may be delivered to a cell where genome-editing is desired. The RNA-guided nucleases may be expressed in a cell where genome-editing is desired with the chemically modified guide RNAs delivered separately. For example, the RNA-guided nucleases may be expressed from a polynucleotide such as a vector or a synthetic mRNA. The vector may be a viral vector, including, be not limited to, an adeno-associated virus (AAV) vector or a lentivirus (LV) vector.

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following examples, which are included for purposes of illustration only and are not intended to be limiting.

### EXAMPLES

### Example 1—Synthesis of Chemically Modified crRNA and tracrRNA

crRNAs and tracrRNAs were synthesized at 1 umole scale on an Applied Biosystems 394 DNA synthesizer. BTT (0.25 M in acetonitrile, ChemGenes) was used as activator. 0.05 M iodine in pyridine:water (9:1) (TEDIA) was used as oxidizer. DDTT (0.1 M, ChemGenes) was used as sulfurizing agent. 3% TCA in DCM (TEDIA) was used as deblock solution. RNAs were grown on 1000 Å CPG functionalized with Unylinker (˜42 μmol/g). RNA and 2′-OMe phosphoramidites

(ChemGenes) were dissolved in acetonitrile to 0.15 M: the coupling time was 10 min for each base. The nucleobases were deprotected with a 3:1 NH4OH: EtOH solution for 48 hours at room temperature. Deprotection of the TBDMS group was achieved with DMSO:NEt3·3HF (4:1) solution (500 μL) at 65 ° C.for 3 hours. RNA oligonucleotides were then recovered by precipitation in 3M NaOAc (25 μL) and n-BuOH (1 mL), and the pellet was washed with cold 70% EtOH and resuspended in 1 mL RNase-free water.

Purification of the crRNAs and tracrRNAs were carried out by high performance liquid chromatography using a 1260 infinity system with an Agilent PL-SAX 1000 Å column (150×7.5 mm, 8 μm). Buffer A: 30% acetonitrile in water; Buffer B: 30% acetonitrile in IM NaClO4 (aq). Excess salt was removed with a Sephadex Nap-10 column.

crRNAs and tracrRNAs were analyzed on an Agilent 6530 Q-TOF LC/MS system with electrospray ionization and time of flight ion separation in negative ionization mode. The data were analyzed using Agilent Mass Hunter software. Buffer A: 100mM hexafluoroisopropanol with 9mM triethylamine in water; Buffer B: 100mM hexafluoroisopropanol with 9 mM trimethylamine in methanol.

Synthesis of exNA-containing nucleotides and exNA-containing oligonucleotides (e.g., crRNAs and tracrRNAs) is described in further detail in WO2021242883, WO2021195533, and WO2020198509, each of which is incorporated herein by reference.

The crRNAs used in the Examples are recited below in Table 7. Table 4 above recites tracrRNAs used in the Examples.

TABLE 7
Exemplary exNA-containing crRNAs.
KEY: rN = RNA, mN = 2′-O-methyl RNA,
fN = 2′-fluoro RNA, NexN = exNA linkage,
N = A, U, G, or C N#N = phosphorothioate
linkage

| Name | Sequence |
|---|---|
| crRNA | mG#mA#mG#mAmCmAmAmAmUmCfAfCfCfUrG#rC#fC |
| 20 | fUrC#mGmGrU#rU#rU#fUfAmGmAmGmCmUmAmU#mG |
| | #mC#Mu (SEQ ID NO: 283) |
| crRNA | mG#mA#mG#mAmCmAmAmAmUmCfAfCfCfUrG#rC#fC |
| Ex20-1 | fUrC#mGmGrU#rU#rU#fUfAmGmAmGmCmUmAmU#mG |
| | #mC#exmU SEQ ID NO: 284) |
| crRNA | mG#mA#mG#mAmCmAmAmAmUmCfAfCfCfUrG#rC#fC |
| Ex20-4 | fUrC#mGmGrU#rU#rU#fUfAmGmAmGmCmUmAexmU# |
| | mG#mC#mU (SEQ ID NO: 285) |
| crRNA | mG#mA#mG#mAmCmAmAmAmUmCfAfCfCfUrG#rC#fC |
| Ex20-5 | fUrC#mGmGrU#rU#rU#fUfAmGmAmGmCexmUmAmU# |
| | mG#mC#mU (SEQ ID NO: 286) |
| crRNA | mG#mA#mG#mAmCmAmAmAmUmCfAfCfCfUrG#rC#fC |
| Ex20-7 | fUrC#mGmGrU#rU#rU#exfUfAmGmAmGmCmUmAmU# |
| | mG#mC#Mu (SEQ ID NO: 287) |
| crRNA | mG#mA#mG#mAmCmAmAmAmUmCfAfCfCfUrG#rC#fC |
| Ex20-8 | fUrC#mGmGrU#rU#exrU#fUfAmGmAmGmCmUmAmU# |
| | mG#mC#mU (SEQ ID NO: 288) |
| crRNA | mG#mA#mG#mAmCmAmAmAmUmCfAfCfCfUrG#rC#fC |
| Ex20-9 | fUrC#mGmGrU#exrU#rU#fUfAmGmAmGmCmUmAmU# |
| | mG#mC#mU (SEQ ID NO: 289) |
| crRNA | mG#mA#mG#mAmCmAmAmAmUmCfAfCfCfUrG#rC#fC |
| Ex20-10 | fUrC#mGmGexrU#rU#rU#fUfAmGmAmGmCmUmAmU# |
| | mG#mC#mU (SEQ ID NO: 290) |
| crRNA | mG#mA#mG#mAmCmAmAmAmUmCfAfCfCfUrG#rC#fC |
| Ex20-12 | exfUrC#mGmGrU#rU#rU#fUfAmGmAmGmCmUmAmU# |
| | mG#mC#mU (SEQ ID NO: 291) |
| crRNA | mG#mA#mG#mAmCmAmAmAmUmCfAfCfCexfUrG#rC# |
| Ex20-13 | fCfUrC#mGmGrU#rU#rU#fUfAmGmAmGmCmUmAmU# |
| | mG#mC#mU (SEQ ID NO: 292) |
| crRNA | mG#mA#mG#mAmCmAmAmAexmUmCfAfCfCfUrG#rC# |
| Ex20-15 | fCfUrC#mGmGrU#rU#rU#fUfAmGmAmGmCmUmAmU# |
| | mG#mC#mU (SEQ ID NO: 293) |

### Example 2—General Methods

Chemically Modified crRNA and tracrRNA Screening Methods

### Cell Culture

Screening was performed in a HEK293T stable cell line expressing the traffic light reporter (TLR) Multi-Cas Variant 1 system (TLR-MCV1). The HEK293T cells were cultured in Dulbecco-modified Eagle's Minimum Essential Medium (DMEM; Life Technologies). DMEM was also supplemented with 10% Fetal Bovine Serum (FBS; Sigma). Cells were grown in a humidified 37° C., 5% CO2 incubator.

### Traffic Light Reporter (TLR) System

The traffic light reporter (TLR) system includes a GFP (containing an insertion), followed by an out-of-frame mCherry. Upon double stranded break induction, a subset of non-homologous end-joining (NHEJ) repair events generate indels that place mCherry in frame, leading to red fluorescence. Detection of the red fluorescence is therefore a readout of editing efficiency. This system was developed and further described in Certo et al. (Nat. Methods 8, 671 (2011)). This system was further developed for testing the modified crRNAs and tracrRNAs of the disclosure. The TLR Multi-Cas Variant 1 system (TLR-MCV1) was created to introduce protospacer adjacent motifs (PAMs) to multiple alternative CRISPR enzymes (Streptococcus pyogenes (SpyCas9), Neisseria meningiditis (NmelCas9 and Nme2Cas9), Campylobacter jejuni (CjeCas9), Staphylococcus aureus (SauCas9), Geobacillus stearothermophilus (GeoCas9), Lachnospiraceae bacterium ND2006 (LbaCas12a), Acidaminococcus sp. (AspCas12a), and Francisella novicida (FnoCas12)). An additional SpyCas9 editing site was introduced as well, producing editing The MCV1a is sites MCV1a and MCV1b. target GAGACAAAUCACCUGCCUCG(SEQ ID NO:294) and the MCV1b target is UUUACCGUAUUCCACGAGGC(SEQ ID NO:295). These overlapping SpyCas9 cleavage sites permit the evaluation of two different crRNA sequences targeting the same position.

### Expression and Purification of Spy-Cas9

The pMCSG7 vector expressing the Cas9 from Streptococcus pyogenes was used. In this construct, the Cas9 also contains three nuclear localization signals (NLSs). Rosetta DE3 strain of Escherichia coli was transformed with the 3xNLS-Spy Cas9 construct. For expression and purification of 3xNLS-SpyCas9, a previously described protocol was used (Jinek et al. Science, 337: 816 (2012)). The bacterial culture was grown at 37° C. until an OD600 of 0.6 was reached. Then, the bacterial culture was cooled to 18° C., and 1 mM Isopropyl β-D-1-thiogalactopyranoside

(IPTG: Sigma) was added to induce protein expression. Cells were grown overnight for 16-20 hours.

The bacterial cells were harvested and resuspended in Lysis Buffer [50 mM Tris-HCl (pH 8.0), 5 mM imidazole]. 10 μg/mL of Lysozyme (Sigma) was then added to the mixture and incubated for 30 minutes at 4° C. This was followed by the addition of 1x× HALT Protease Inhibitor Cocktail (ThermoFisher). The bacterial cells were then sonicated and centrifuged for 30 minutes at 18,000 rpm. The supernatant was then subjected to Nickel affinity chromatography. The elution fractions containing the Spy Cas9 were then further purified using cation exchange chromatography using a 5 mL HiTrap S HP column (GE). This was followed by a final round of purification by size-exclusion chromatography using a Superdex-200 column (GE). The purified protein was concentrated and flash frozen for subsequent use.

### Transfection of HEK293T Cells

The HEK293T cells were nucleofected using the Neon transfection system (ThermoFisher) according to the manufacturer's protocol. Briefly, 20 picomoles of 3xNLS-SpyCas9 was mixed with 25 picomoles of crRNA:tracrRNA in buffer R (ThermoFisher) and incubated at room temperature for 20-30 minutes. This Cas9 RNP complex was then mixed with approximately 100,000 cells which were already resuspended in buffer R. This mixture was nucleofected with a 10 μL Neon tip and then plated in 24-well plates containing 500 μL of DMEM and 10% FBS. The cells were stored in a humidified 37° C. and 5% CO2 incubator for 2-3 days.

### Flow Cytometry Analysis

The nucleofected HEK293T cells were analyzed on MACSQuant® VYB from Miltenyi Biotec. For mCherry detection, the yellow laser (561 nm) was used for excitation and 615/20 nm filter used to detect emission. At least 20,000 events were recorded and the subsequent analysis was performed using FlowJoR ¥10.4.1.

Cells were first sorted based on forward and side scattering (FSC-A vs SSC-A) to eliminate debris. Cells were then gated using FSC-A and FSC-H to select single cells. Finally, mCherry signal was used to select for mCherry-expressing cells. The percent of cells expressing mCherry was calculated and reported in this application as a measure of Cas9-based genome editing.

### Example 3 —Chemically Modified crRNA and tracrRNA Sequences Containing exNA Intersubunit Linkages

Prior work demonstrated that several chemical modification patterns of crRNA and tracrRNA were capable of being active while increasing serum stability (WO2019183000 and WO2021231606, each of which is incorporated herein by reference). The modified crRNAs created previously were C1 to C134 and the modified tracrRNA created previously were T1 to T116 (see Table 1 and Table 2 above). In an effort to further modify the guide RNA while retaining activity, the crRNA portion and tracrRNA portion were modified with the exNA intersubunit linkage. The exNA intersubunit linkage may improve serum stability and increase nuclease resistance of the guide RNA without sacrificing gene editing efficiency.

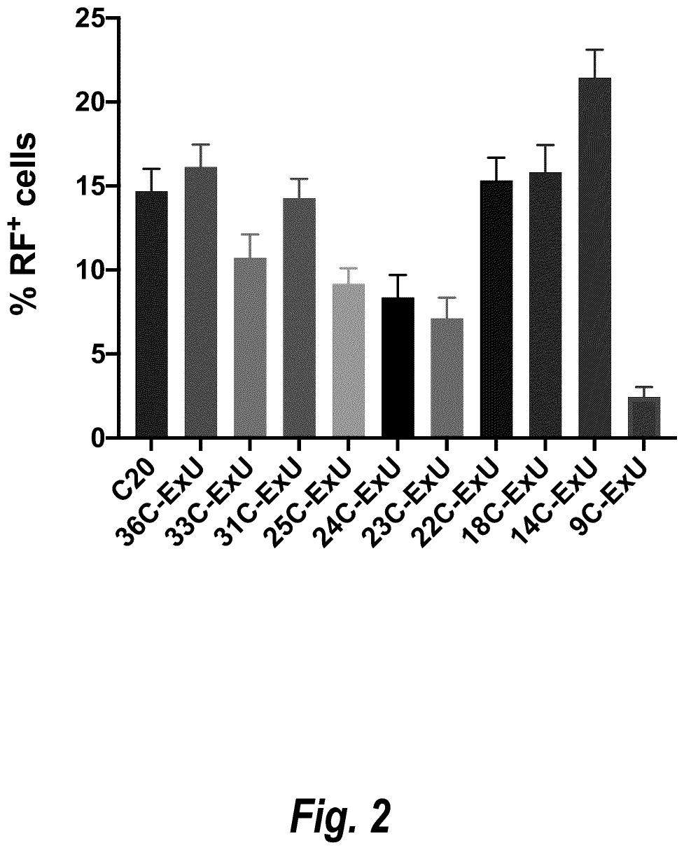

Using the chemical modification pattern of crRNA 20 as a template, exNA intersubunit linkages were added at various positions (see Table 6). The different exNA-crRNAs were paired with tracrRNA 2. Five pmol of an RNP containing the guide RNA and Cas9 was transfected into the TLR-MCV1 reporter targeting MCV1a, as described above. As shown in FIG. 2, all of the tested exNA-crRNAs retained gene editing activity, with several retaining or possessing higher activity than C20, which does not contain exNA intersubunit linkages. ExNA intersubunit linkages were placed in the guide region of the crRNA (typically the first 20 nucleotides from the 5′ end) and the repeat region that hybridizes with the anti-repeat region of the tracrRNA.

Surprisingly, guide RNAs containing the exNA intersubunit linkage within the target-specific guide region of the guide RNA (i.e., the first 20 nucleotides from the 5′ end of the crRNA portion) retained gene-editing activity. In certain instances, the activity was superior to the chemically modified, non-exNA-containing crRNA 20 (C20).

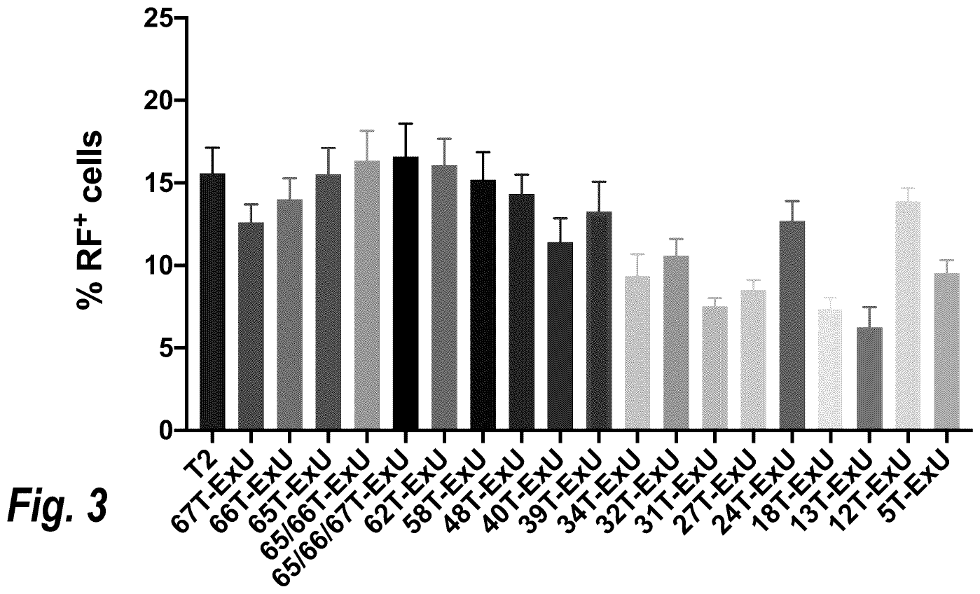

Using the chemical modification pattern of tracrRNA 2 as a template, exNA intersubunit linkages were added at various positions (see Table 4). The different exNA-tracrRNAs were paired with crRNA 20. Five pmol of an RNP containing the guide RNA and Cas9 was transfected into the TLR-MCV1 reporter targeting MCV1a, as described above. As shown in FIG. 3, all of the tested exNA-tracrRNAs retained gene editing activity. The guide RNA retained gene editing activity when exNA intersubunit linkages were placed in the anti-repeat region, stem loop 1, stem loop 2, and stem loop 3 of the tracrRNA.

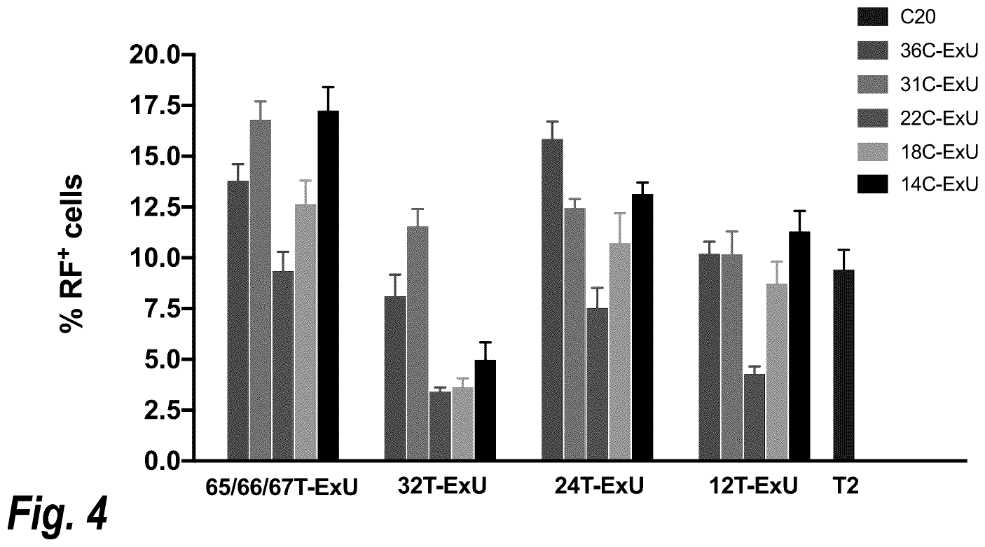

ExNA-modified crRNAs were next paired with exNA-modified tracrRNAs. As shown in FIG. 4, the dual guide with exNA linkages in both the crRNA portion and tracrRNA portion displayed similar to superior editing compared to the non-exNA modified guide (C20/T2 combination).

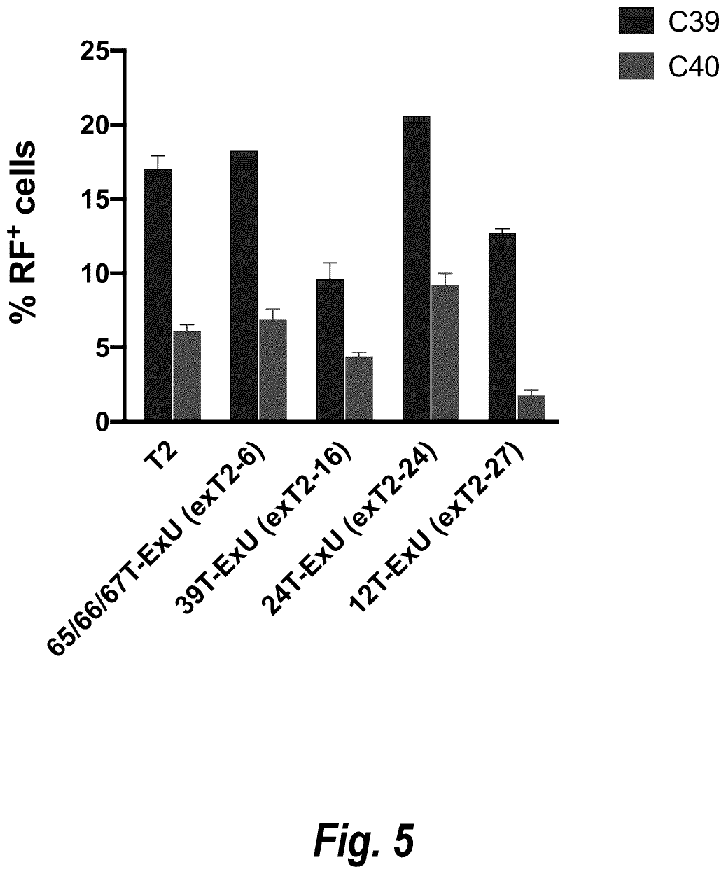

Additional exNA-modified tracrRNAs were paired with crRNA C39 and C40. As shown in FIG. 5, editing was similar to superior editing compared to the non-exNA modified guide (C39/T2 or C40/T2 combination).

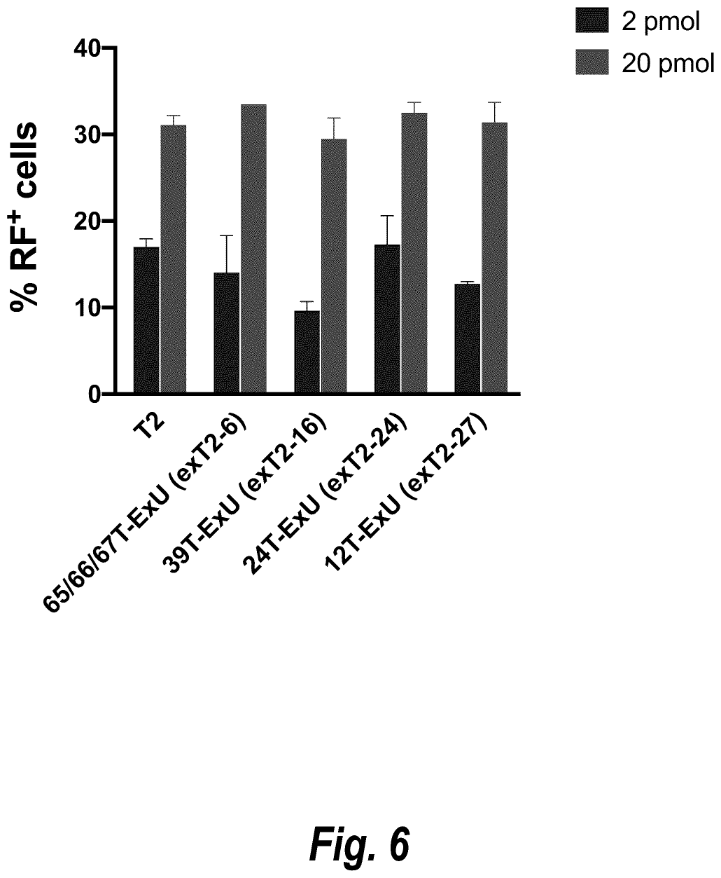

The C39 exNA-modified tracrRNA combination was next tested in an RNP at 2 and 20 pmol. As shown in FIG. 6, both amounts of RNP were capable of genome editing.

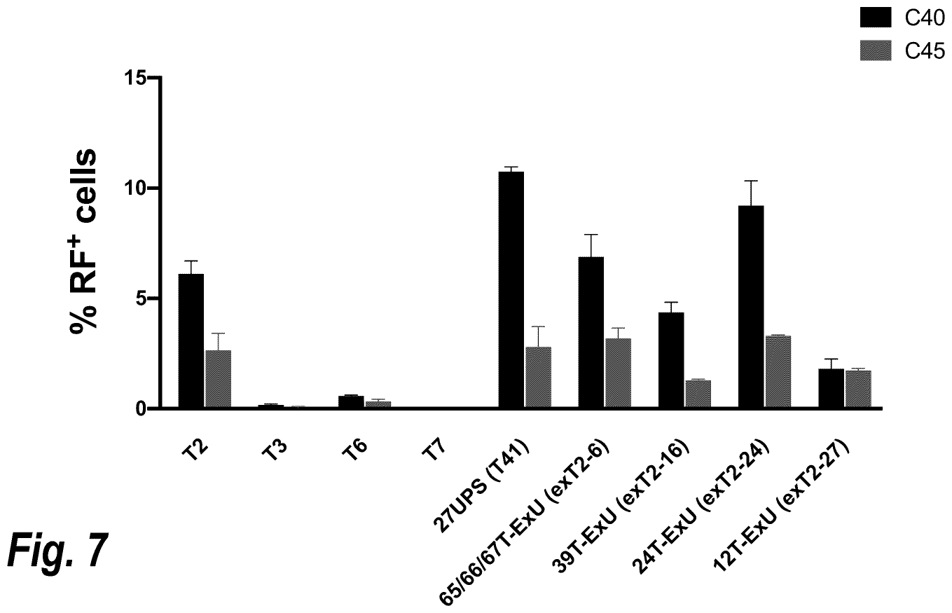

As shown in FIG. 7, the exNA-modified tracrRNA portions, when paired with C40 or C45, displayed better editing compared to non-exNA modified tracrRNAs.

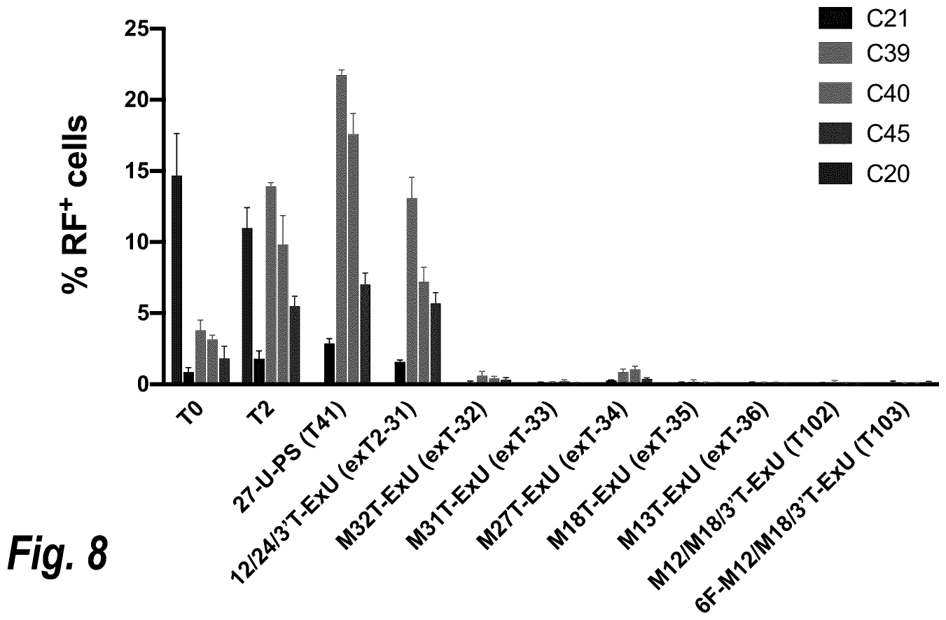

As shown in FIG. 8, editing efficiencies of several exNA-containing tracrRNAs paired with crRNAs C20, C21, C39, C40, and C45 were tested.

1. A chemically modified guide RNA comprising:
(a) a crRNA portion comprising (i) a guide sequence capable of hybridizing to a target polynucleotide sequence, and (ii) a repeat sequence; and
(b) a tracrRNA portion comprising an anti-repeat nucleotide sequence that is complementary to the repeat sequence, wherein the crRNA portion comprises at least one exNA intersubunit linkage.

2. The chemically modified guide RNA of claim 1, wherein the at least one exNA intersubunit linkage is in the guide sequence.

3. (canceled)

4. The chemically modified guide RNA of claim 1, wherein:
crRNA portion comprises the at least one exNA intersubunit linkage between one or more of positions 35 to 36, 32 to 33, 30 to 31, 24 to 25, 23 to 24, 22 to 23, 21 to 22, 17 to 18, 13 to 14, and 8 to 9, from the 5′ end of the crRNA portion; or
crRNA portion comprises the at least one exNA intersubunit linkage between one or more of positions 35 to 36, 32 to 33, 30 to 31, 24 to 25, 23 to 24, 22 to 23, 21 to 22, 17 to 18, 13 to 14, and 8 to 9, from the 5′ end of the crRNA portion set forth in NNNNNNNNNNNNNNNNNNNNGUUUUAGAGCUAUGCU (SEQ ID NO: 1), wherein “N” corresponds to a nucleotide selected from the group consisting of A, U, G, and C.

5. (canceled)

6. The chemically modified guide RNA of anyclaim 1, wherein the at least one exNA intersubunit linkage comprises the intersubunit linkage of Formula Ia: 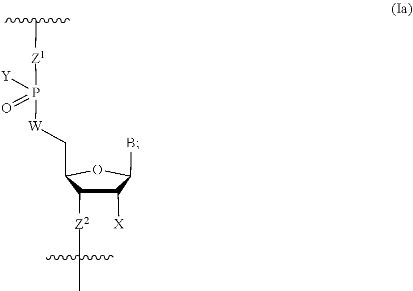
wherein:
B is a base moiety;
W is O or O(CH₂)ₙ¹, wherein n¹ is an integer between 1 and 10;
X is selected from the group consisting of H, OH, OR¹, R¹, F, Cl, Br, I, SH, SR¹, NH₂, NHR¹, NR¹₂, and COOR¹;
Y is selected from the group consisting of O⁻, OH, OR², NH⁻, NH₂, NR²₂, BH₃, S⁻, R², and SH;
Z¹ is O or O(CH₂)n² wherein n² is an integer between 1 and 10;
Z² is O or O(CH₂)n³ wherein n³ is an integer between 1 and 10;
R¹ is a substituted or unsubstituted C₁-C₆ alkyl, alkenyl, alkynyl, aryl, or mixtures thereof; and
R² is a substituted or unsubstituted C₁-C₆ alkyl, alkenyl, alkynyl, aryl, or mixtures thereof, wherein:
Z₁ is O(CH₂)n² and W is O, [or] Z¹ is O and W is O(CH₂)ₙ¹, or Z₁ is O(CH₂)n² and W is O(CH₂)ₙ¹.

7-14. (canceled)

15. The chemically modified guide RNA of claim 1, wherein the crRNA portion and/or the tracrRNA portion further comprise at least one modified nucleotide selected from a modification of a ribose group, a phosphate group, a nucleobase, or a combination thereof.

16-23. (canceled)

24. The chemically modified guide RNA of claim 1, comprising a crRNA portion modification pattern selected from the group consisting of:

| Name | |
|---|---|
| (Alter- | |
| nate | |
| Name) | Pattern |
| crRNA | mN#mN#mN#mNmNmNmNmNmNmNfNfNfNfNrN#rN#f |
| Ex20-1 | NfNrN#mNmGrU#rU#rU#fUfAmGmAmGmCmUmAmU# |
| (36C- | mG#mC#exmU (SEQ ID NO: 5); |
| ExU) | |
| crRNA | mN#mN#mN#mNmNmNmNmNmNmNfNfNfNfNrN#rN#f |
| Ex20-4 | NfNrN#mNmGrU#rU#rU#fUfAmGmAmGmCmUmAexmU |
| (33C- | #mG#mC#mU (SEQ ID NO: 6); |
| ExU) | |
| crRNA | mN#mN#mN#mNmNmNmNmNmNmNfNfNfNfNrN#rN#f |
| Ex20-5 | NfNrN#mNmGrU#rU#rU#fUfAmGmAmGmCexmUmAmU |
| (31C- | #mG#mC#mU (SEQ ID NO: 7); |
| ExU) | |
| crRNA | mN#mN#mN#mNmNmNmNmNmNmNfNfNfNfNrN#rN#f |
| Ex20-7 | NfNrN#mNmGrU#rU#rU#exfUfAmGmAmGmCmUmAmU |
| (25C- | #mG#mC#mU (SEQ ID NO: 8); |
| ExU) | |
| crRNA | mN#mN#mN#mNmNmNmNmNmNmNfNfNfNfNrN#rN#f |
| Ex20-8 | NfNrN#mNmGrU#rU#exrU#fUfAmGmAmGmCmUmAmU |
| (24C- | #mG#mC#mU (SEQ ID NO: 9); |
| ExU) | |
| crRNA | mN#mN#mN#mNmNmNmNmNmNmNfNfNfNfNrN#rN#f |
| Ex20-9 | NfNrN#mNmGrU#exrU#rU#fUfAmGmAmGmCmUmAmU |
| (23C- | #mG#mC#mU (SEQ ID NO: 10); |
| ExU) | |
| crRNA | mN#mN#mN#mNmNmNmNmNmNmNfNfNfNfNrN#rN#f |
| Ex20-10 | NfNrN#mNmGexrU#rU#rU#fUfAmGmAmGmCmUmAmU |
| (22C- | #mG#mC#mU (SEQ ID NO: 11); |
| ExU) | |
| crRNA | mN#mN#mN#mNmNmNmNmNmNmNfNfNfNfNrN#rN#f |
| Ex20-12 | NexfNrN#mNmGrU#rU#rU#fUfAmGmAmGmCmUmAmU |
| (18C- | #mG#mC#Mu (SEQ ID NO: 12); |
| ExU) | |
| crRNA | mN#mN#mN#mNmNmNmNmNmNmNfNfNfNexfNrN#rN# |
| Ex20-13 | fNfNrN#mNmGrU#rU#rU#fUfAmGmAmGmCmUmAmU# |
| (14C- | mG#mC#Mu (SEQ ID NO: 13); and |
| ExU) | |
| crRNA | mN#mN#mN#mNmNmNmNmNexmNmNfNfNfNfNrN#rN# |
| Ex20-15 | fNfNrN#mNmGrU#rU#rU#fUfAmGmAmGmCmUmAmU# |
| (9C- | mG#mC#mU (SEQ ID NO: 14), |
| ExU) | |

wherein:
rNis an RNA;
mN is a 2′-O-methyl RNA;
fN is a 2′-fluoro RNA;
NexN is an exNA linkage;
Nis A, U, G, or C; and
N#N is a phosphorothioate linkage.

25. The chemically modified guide of claim 1, comprising a tracrRNA portion modification pattern selected from any one of tracrRNA 1 to tracrRNA 116 of Table 2 or any one of tracrRNA 2-1 (67T-ExU), tracrRNA 2-2 (66T-ExU), tracrRNA 2-3 (65T-ExU), tracrRNA 2-5 (66/67T-ExU), tracrRNA 2-6 (65/66/67T-ExU), tracrRNA 2-8 (62T-ExU), tracrRNA 2-10 (58T-ExU), tracrRNA 2-14 (48T-ExU), tracrRNA 2-15 (40T-ExU), tracrRNA 2-16 (39T-ExU), tracrRNA 2-19 (34T-ExU), tracrRNA 2-20 (32T-ExU), tracrRNA 2-21 (31T-ExU), tracrRNA 2-23 (27T-ExU), tracrRNA 2-24 (24T-ExU), tracrRNA 2-25 (18T-ExU), tracrRNA 2-26 (13T-ExU), tracrRNA 2-27 (12T-ExU), tracrRNA 2-30 (5T-ExU), tracrRNA 2-31 (12/24/3′T-ExU), tracrRNA 2-32 (M32T-ExU), tracrRNA 2-33 (M31T-ExU), tracrRNA 2-34 (M27T-ExU), tracrRNA 2-35 (M18T-ExU), tracrRNA 2-36 (M13T-ExU), tracrRNA 102 (M12/M18/3′T-ExU), and tracrRNA 103 (6F-M12/M18/3′T-ExU) of Table 4.

26. A chemically modified guide RNA comprising:
(a) a crRNA portion comprising (i) a guide sequence capable of hybridizing to a target polynucleotide sequence, and (ii) a repeat sequence; and
(b) a tracrRNA portion comprising an anti-repeat nucleotide sequence that is complementary to the repeat sequence, wherein the tracrRNA portion comprises at least one exNA intersubunit linkage.

27. The chemically modified guide RNA of claim 26, wherein the at least one exNA intersubunit linkage is in a tracrRNA portion stem-loop.

28. (canceled)

29. The chemically modified guide RNA of claim 26, wherein;
tracrRNA portion comprises the at least one exNA intersubunit linkage between one or more of positions 66 to 67, 65 to 66, 64 to 65, 61 to 62, 57 to 58, 47 to 48, 39 to 40, 38 to 39, 33 to 34, 31 to 32, 30 to 31, 26 to 27, 23 to 24, 17 to 18, 12 to 13, 11 to 12, and 4 to 5, from the 5′ end of the tracrRNA portion; or
tracrRNA portion comprises the at least one exNA intersubunit linkage between one or more of positions 66 to 67, 65 to 66, 64 to 65, 61 to 62, 57 to 58, 47 to 48, 39 to 40, 38 to 39, 33 to 34, 31 to 32, 30 to 31, 26 to 27, 23 to 24, 17 to 18, 12 to 13, 11 to 12, and 4 to 5, from the 5′ end of the tracrRNA portion set forth in SEQ ID NO: 2.

30. (canceled)

31. The chemically modified guide RNA of claim 26, wherein the at least one exNA intersubunit linkage comprises the intersubunit linkage of Formula Ia: 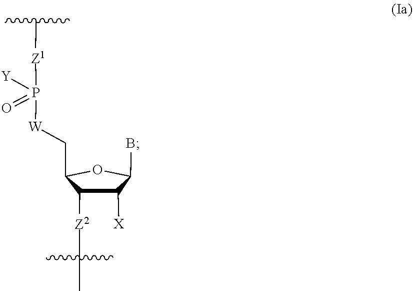
wherein:
B is a base moiety;
W is O or O(CH₂)ₙ¹, wherein n¹ is an integer between 1 and 10;
X is selected from the group consisting of H, OH, OR¹, R¹, F, Cl, Br, I, SH, SR¹, NH₂, NHR¹, NR¹₂, and COOR¹;
Y is selected from the group consisting of O⁻, OH, OR², NH⁻, NH₂, NR²₂, BH₃, S⁻, R², and SH;
Z¹ is O or O(CH₂)ₙ² wherein n² is an integer between 1 and 10;
Z² is O or O(CH₂)ₙ³ wherein n³ is an integer between 1 and 10;
R¹ is a substituted or unsubstituted C₁-C₆ alkyl, alkenyl, alkynyl, aryl, or mixtures thereof; and
R² is a substituted or unsubstituted C₁-C₆ alkyl, alkenyl, alkynyl, aryl, or mixtures thereof, wherein:
Z₁ is O(CH₂)ₙ² and W is O, Z₁ is O and W is O(CH₂)ₙ¹, or Z₁ is O(CH₂)ₙ² and W is O(CH₂)ₙ¹.

32-39. (canceled)

40. The chemically modified guide RNA of claim 26, wherein the crRNA portion and/or the tracrRNA portion further comprise at least one modified nucleotide selected from a modification of a ribose group, a phosphate group, a nucleobase, and a combination thereof.

41-48. (canceled)

49. The chemically modified guide RNA of claim 26, comprising a tracrRNA portion modification pattern selected from the group consisting of:

| Name | |
|---|---|
| (Alter- | |
| nate | |
| Name) | Pattern |
| tracrRNA | mA#mG#mC#mAmUmAmGmCmAmAmGrUrUmArAmA |
| 2 | mArUmAmAmGmGrCrUmArGrUrCmCrGrUrUmAmUmC |
| | mAmAmCmUmUmGmAmAmAmAmAmGmUmGmGmCm |
| | AmCmCmGmAmGmUmCmGmGmUmGmC#mU#mU#mU |
| | (SEQ ID NO: 15); |
| tracrRNA | mA#mG#mC#mAmUmAmGmCmAmAmGrUrUmArAmA |
| 2-1 | mArUmAmAmGmGrCrUmArGrUrCmCrGrUrUmAmUmC |
| (67T- | mAmAmCmUmUmGmAmAmAmAmAmGmUmGmGmCm |
| ExU) | AmCmCmGmAmGmUmCmGmGmUmGmC#mU#mU#exm |
| | U (SEQ ID NO: 16); |
| tracrRNA | mA#mG#mC#mAmUmAmGmCmAmAmGrUrUmArAmA |
| 2-2 | mArUmAmAmGmGrCrUmArGrUrCmCrGrUrUmAmUmC |
| (66T- | mAmAmCmUmUmGmAmAmAmAmAmGmUmGmGmCm |
| ExU) | AmCmCmGmAmGmUmCmGmGmUmGmC#mU#exmU#m |
| | U (SEQ ID NO: 17); |
| tracrRNA | mA#mG#mC#mAmUmAmGmCmAmAmGrUrUmArAmA |
| 2-3 | mArUmAmAmGmGrCrUmArGrUrCmCrGrUrUmAmUmC |
| (65T- | mAmAmCmUmUmGmAmAmAmAmAmGmUmGmGmCm |
| ExU) | AmCmCmGmAmGmUmCmGmGmUmGmC#exmU#mU#m |
| | U (SEQ ID NO: 18); |
| tracrRNA | mA#mG#mC#mAmUmAmGmCmAmAmGrUrUmArAmA |
| 2-5 | mArUmAmAmGmGrCrUmArGrUrCmCrGrUrUmAmUmC |
| (66/67T- | mAmAmCmUmUmGmAmAmAmAmAmGmUmGmGmCm |
| ExU) | AmCmCmGmAmGmUmCmGmGmUmGmC#mU#exmU#e |
| | xmU (SEQ ID NO: 19); |
| tracrRNA | mA#mG#mC#mAmUmAmGmCmAmAmGrUrUmArAmA |
| 2-6 | mArUmAmAmGmGrCrUmArGrUrCmCrGrUrUmAmUmC |
| (65/66/ | mAmAmCmUmUmGmAmAmAmAmAmGmUmGmGmCm |
| 67T- | AmCmCmGmAmGmUmCmGmGmUmGmC#exmU#exmU |
| ExU) | #exmU (SEQ ID NO: 20); |
| tracrRNA | mA#mG#mC#mAmUmAmGmCmAmAmGrUrUmArAmA |
| 2-8 | mArUmAmAmGmGrCrUmArGrUrCmCrGrUrUmAmUmC |
| (62T- | mAmAmCmUmUmGmAmAmAmAmAmGmUmGmGmCm |
| ExU) | AmCmCmGmAmGmUmCmGmGexmUmGmC#mU#mU#m |
| | U (SEQ ID NO: 21); |
| tracrRNA | mA#mG#mC#mAmUmAmGmCmAmAmGrUrUmArAmA |
| 2-10 | mArUmAmAmGmGrCrUmArGrUrCmCrGrUrUmAmUmC |
| (58T- | mAmAmCmUmUmGmAmAmAmAmAmGmUmGmGmCm |
| ExU) | AmCmCmGmAmGexmUmCmGmGmUmGmC#mU#mU#m |
| | U (SEQ ID NO: 22); |
| tracrRNA | mA#mG#mC#mAmUmAmGmCmAmAmGrUrUmArAmA |
| 2-14 | mArUmAmAmGmGrCrUmArGrUrCmCrGrUrUmAmUmC |
| (48T- | mAmAmCmUmUmGmAmAmAmAmAmGexmUmGmGm |
| ExU) | CmAmCmCmGmAmGmUmCmGmGmUmGmC#mU#mU# |
| | mU (SEQ ID NO: 23); |
| tracrRNA | mA#mG#mC#mAmUmAmGmCmAmAmGrUrUmArAmA |
| 2-15 | mArUmAmAmGmGrCrUmArGrUrCmCrGrUrUmAmUmC |
| (40T- | mAmAmCmUexmUmGmAmAmAmAmAmGmUmGmGm |
| ExU) | CmAmCmCmGmAmGmUmCmGmGmUmGmC#mU#mU# |
| | mU (SEQ ID NO: 24); |
| tracrRNA | mA#mG#mC#mAmUmAmGmCmAmAmGrUrUmArAmA |
| 2-16 | mArUmAmAmGmGrCrUmArGrUrCmCrGrUrUmAmUmC |
| (39T- | mAmAmCexmUmUmGmAmAmAmAmAmGmUmGmGm |
| ExU) | CmAmCmCmGmAmGmUmCmGmGmUmGmC#mU#mU# |
| | mU (SEQ ID NO: 25); |
| tracrRNA | mA#mG#mC#mAmUmAmGmCmAmAmGrUrUmArAmA |
| 2-19 | mArUmAmAmGmGrCrUmArGrUrCmCrGrUrUmAexmUm |
| (34T- | CmAmAmCmUmUmGmAmAmAmAmAmGmUmGmGmC |
| ExU) | mAmCmCmGmAmGmUmCmGmGmUmGmC#mU#mU#m |
| | U (SEQ ID NO: 26); |
| tracrRNA | mA#mG#mC#mAmUmAmGmCmAmAmGrUrUmArAmA |
| 2-20 | mArUmAmAmGmGrCrUmArGrUrCmCrGrUexrUmAmUm |
| (32T- | CmAmAmCmUmUmGmAmAmAmAmAmGmUmGmGmC |
| ExU) | mAmCmCmGmAmGmUmCmGmGmUmGmC#mU#mU#m |
| | U (SEQ ID NO: 27); |
| tracrRNA | mA#mG#mC#mAmUmAmGmCmAmAmGrUrUmArAmA |
| 2-21 | mArUmAmAmGmGrCrUmArGrUrCmCrGexrUrUmAmUm |
| (31T- | CmAmAmCmUmUmGmAmAmAmAmAmGmUmGmGmC |
| ExU) | mAmCmCmGmAmGmUmCmGmGmUmGmC#mU#mU#m |
| | U (SEQ ID NO: 28); |
| tracrRNA | mA#mG#mC#mAmUmAmGmCmAmAmGrUrUmArAmA |
| 2-23 | mArUmAmAmGmGrCrUmArGexrUrCmCrGrUrUmAmUm |
| (27T- | CmAmAmCmUmUmGmAmAmAmAmAmGmUmGmGmC |
| ExU) | mAmCmCmGmAmGmUmCmGmGmUmGmC#mU#mU#m |
| | U (SEQ ID NO: 29); |
| tracrRNA | mA#mG#mC#mAmUmAmGmCmAmAmGrUrUmArAmA |
| 2-24 | mArUmAmAmGmGrCexrUmArGrUrCmCrGrUrUmAmUm |
| (24T- | CmAmAmCmUmUmGmAmAmAmAmAmGmUmGmGmC |
| ExU) | mAmCmCmGmAmGmUmCmGmGmUmGmC#mU#mU#m |
| | U (SEQ ID NO: 30); |
| tracrRNA | mA#mG#mC#mAmUmAmGmCmAmAmGrUrUmArAmA |
| 2-25 | mAexrUmAmAmGmGrCrUmArGrUrCmCrGrUrUmAmUm |
| (18T-ExU) | CmAmAmCmUmUmGmAmAmAmAmAmGmUmGmGmC |
| | mAmCmCmGmAmGmUmCmGmGmUmGmC#mU#mU#m |
| | U (SEQ ID NO: 31); |
| tracrRNA | mA#mG#mC#mAmUmAmGmCmAmAmGrUexrUmArAm |
| 2-26 | AmArUmAmAmGmGrCrUmArGrUrCmCrGrUrUmAmUm |
| (13T-ExU) | CmAmAmCmUmUmGmAmAmAmAmAmGmUmGmGmC |
| | mAmCmCmGmAmGmUmCmGmGmUmGmC#mU#mU#m |
| | U (SEQ ID NO: 32); |
| tracrRNA | mA#mG#mC#mAmUmAmGmCmAmAmGexrUrUmArAm |
| 2-27 | AmArUmAmAmGmGrCrUmArGrUrCmCrGrUrUmAmUm |
| (12T-ExU) | CmAmAmCmUmUmGmAmAmAmAmAmGmUmGmGmC |
| | mAmCmCmGmAmGmUmCmGmGmUmGmC#mU#mU#m |
| | U (SEQ ID NO: 33); |
| tracrRNA | mA#mG#mC#mAexmUmAmGmCmAmAmGrUrUmArAm |
| 2-30 | AmArUmAmAmGmGrCrUmArGrUrCmCrGrUrUmAmUm |
| (5T-ExU) | CmAmAmCmUmUmGmAmAmAmAmAmGmUmGmGmC |
| | mAmCmCmGmAmGmUmCmGmGmUmGmC#mU#mU#m |
| | U (SEQ ID NO: 34); |
| tracrRNA | mA#mG#mC#mAmUmAmGmCmAmAmGexrUrUmArAm |
| 2-31 | AmArUmAmAmGmGrCexrUmArGrUrCmCrGrUrUmAmU |
| (12/24/ | mCmAmAmCmUmUmGmAmAmAmAmAmGmUmGmGm |
| 3′T- | CmAmCmCmGmAmGmUmCmGmGmUmGmC#exmU#ex |
| ExU) | mU#exmU (SEQ ID NO: 35); |
| tracrRNA | mA#mG#mC#mAmUmAmGmCmAmAmGrUrUmArAmA |
| 2-32 | mArUmAmAmGmGrCrUmArGrUrCmCrGrUexmUmAmU |
| (M32T- | mCmAmAmCmUmUmGmAmAmAmAmAmGmUmGmGm |
| ExU) | CmAmCmCmGmAmGmUmCmGmGmUmGmC#mU#mU# |
| | mU (SEQ ID NO: 36); |
| tracrRNA | mA#mG#mC#mAmUmAmGmCmAmAmGrUrUmArAmA |
| 2-33 | mArUmAmAmGmGrCrUmArGrUrCmCrGexmUrUmAmU |
| (M31T- | mCmAmAmCmUmUmGmAmAmAmAmAmGmUmGmGm |
| ExU) | CmAmCmCmGmAmGmUmCmGmGmUmGmC#mU#mU# |
| | mU (SEQ ID NO: 37); |
| tracrRNA | mA#mG#mC#mAmUmAmGmCmAmAmGrUrUmArAmA |
| 2-34 | mArUmAmAmGmGrCrUmArGexmUrCmCrGrUrUmAmU |
| (M27T- | mCmAmAmCmUmUmGmAmAmAmAmAmGmUmGmGm |
| ExU) | CmAmCmCmGmAmGmUmCmGmGmUmGmC#mU#mU# |
| | mU (SEQ ID NO: 38); |
| tracrRNA | mA#mG#mC#mAmUmAmGmCmAmAmGrUrUmArAmA |
| 2-35 | mAexmUmAmAmGmGrCrUmArGrUrCmCrGrUrUmAmU |
| (M18T- | mCmAmAmCmUmUmGmAmAmAmAmAmGmUmGmGm |
| ExU) | CmAmCmCmGmAmGmUmCmGmGmUmGmC#mU#mU# |
| | mU (SEQ ID NO: 39); |
| tracrRNA | mA#mG#mC#mAmUmAmGmCmAmAmGrUexmUmArAm |
| 2-36 | AmArUmAmAmGmGrCrUmArGrUrCmCrGrUrUmAmUm |
| (M13T- | CmAmAmCmUmUmGmAmAmAmAmAmGmUmGmGmC |
| ExU) | mAmCmCmGmAmGmUmCmGmGmUmGmC#mU#mU#m |
| | U (SEQ ID NO: 40; |
| tracrRNA | mA#mG#mC#mAmUmAmGmCmAmAmGexmUrUmArAm |
| 102 | AmAexmUmAmAmGmGrCrUmArGrUrCmCrGrUrUmAm |
| (M12/ | UmCmAmAmCmUmUmGmAmAmAmAmAmGmUmGmG |
| M18/ | mCmAmCmCmGmAmGmUmCmGmGmUmGmC#exmU#e |
| 3′T- | xmU#exmU (SEQ ID NO: 41); and |
| ExU) | |
| tracrRNA | mA#mG#mC#mAmUmAmGmCmAmAmGexmUrUmAfAm |
| 103 (6F- | AmAexmUmAmAmGmGfCrUmAfGrUfCmCfGrUfUmAm |
| M12/ | UmCmAmAmCmUmUmGmAmAmAmAmAmGmUmGmG |
| M18/ | mCmAmCmCmGmAmGmUmCmGmGmUmGmC#exmU#e |
| 3′T- | xmU#exmU (SEQ ID NO: 42), |
| ExU) | |

wherein:
IN is an RNA;
mN is a 2′-O-methyl RNA;
fN is a 2′-fluoro RNA;
NexN is an exNA linkage;
Nis A. U. G, or C; and
N#N is a phosphorothioate linkage.

50. The chemically modified guide of claim 26, comprising a crRNA portion modification pattern of any one of crRNA 1 to crRNA 134 of Table 1 or a crRNA portion modification pattern of any one of crRNA Ex20-1, crRNA Ex20-4, crRNA Ex20-5, crRNA Ex20-7, crRNA Ex20-8, crRNA Ex20-9, crRNA Ex20-10, crRNA Ex20-12, crRNA Ex20-13, and crRNA Ex20-15 of Table 3.

51. The chemically modified guide RNA of claim 1, further comprising at least one moiety conjugated to the guide RNA.

52. The chemically modified guide RNA of claim 51, wherein;
the at least one moiety is conjugated to at least one of the 5′ end of the crRNA portion, the 3′ end of the crRNA portion, the 5′ end of the tracrRNA portion, and the 3′ end of the tracrRNA portion;
the at least one moiety increases cellular uptake of the guide RNA;
the at least one moiety promotes specific tissue distribution of the guide RNA;
the at least one moiety is selected from the group consisting of fatty acids, steroids, secosteroids, lipids, gangliosides analogs, nucleoside analogs, endocannabinoids, vitamins, receptor ligands, peptides, aptamers, and alkyl chains;
the at least one moiety is selected from the group consisting of cholesterol, docosahexaenoic acid (DHA), docosanoic acid (DCA), lithocholic acid (LA), GalNAc, amphiphilic block copolymer (ABC), hydrophilic block copolymer (HBC), poloxamer, Cy5, and Cy3;
the at least one moiety is conjugated to the guide RNA via a linker; or
the at least one moiety is a modified lipid.

53-60. (canceled)

61. The chemically modified guide RNA claim 1, wherein the guide RNA binds to a Cas9 nuclease selected from the group consisting of S. pyogenes Cas9 (SpCas9), S. aureus Cas9 (SaCas9), N. meningitidis Cas9 (NmCas9), C. jejuni Cas9 (CjCas9), and Geobacillus Cas9 (GeoCas9).

62-70. (canceled)

71. A method of altering expression of a target gene in a cell, comprising administering to said cell a genome editing system comprising:
the chemically modified guide RNA of claim 1; and
an RNA-guided nuclease or a polynucleotide encoding an RNA-guided nuclease.

72. The method of claim 71, wherein:
the target gene is in a cell in an organism;
expression of the target gene is knocked out or knocked down;
the sequence of the target gene is modified, edited, corrected or enhanced;
the guide RNA and the RNA-guided nuclease comprise a ribonucleoprotein (RNP) complex;
the RNA-guided nuclease is selected from the group consisting of S. pyogenes Cas9 (SpCas9), S. aureus Cas9 (SaCas9), N. meningitidis Cas9 (NmCas9), C. jejuni Cas9 (CjCas9), and Geobacillus Cas9 (GeoCas9); or
the polynucleotide encoding an RNA-guided nuclease comprises a vector.

73-82. (canceled)

83. A CRISPR genome editing system comprising:
a chemically modified guide RNA of claim 1; and
an RNA-guided nuclease or a polynucleotide encoding an RNA-guided nuclease.

84. The CRISPR genome editing system of claim 83, wherein the RNA-guided nuclease is selected from the group consisting of S. pyogenes Cas9 (SpCas9), S. aureus Cas9 (SaCas9), N. meningitidis Cas9 (NmCas9), C. jejuni Cas9 (CjCas9), and Geobacillus Cas9 (GeoCas9).

85-88. (canceled)


SEQUENCE LISTING (295 sequences)

SEQ ID NO: 1 - RNA, 36 nt, synthetic construct
  source 1..36: mol_type = other RNA
nnnnnnnnnnnnnnnnnnnngttttagagctatgct

SEQ ID NO: 2 - RNA, 67 nt, synthetic construct
  source 1..67: mol_type = other RNA
agcatagcaagttaaaataaggctagtccgttatcaacttgaaaaagtggcaccgagtcggtgcttt

SEQ ID NO: 3 - RNA, 36 nt, synthetic construct
  source 1..36: mol_type = other RNA
nnnnnnnnnnnnnnnnnnnngttttagagcgagcgc

SEQ ID NO: 4 - RNA, 67 nt, synthetic construct
  source 1..67: mol_type = other RNA
gcgctcgcaagttaaaataaggctagtccgttatcaacttgaaaaagtggcaccgagtcggtgcttt

SEQ ID NO: 5 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..4,15..17,19..20,22..25,33..36): mod_base = OTHER; note = Phosphorothioate linkage
  modified_base 35..36: mod_base = OTHER; note = exNA linkage

SEQ ID NO: 6 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..4,15..17,19..20,22..25,33..36): mod_base = OTHER; note = Phosphorothioate linkage
  modified_base 32..33: mod_base = OTHER; note = exNA linkage

SEQ ID NO: 7 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..4,15..17,19..20,22..25,33..36): mod_base = OTHER; note = Phosphorothioate linkage
  modified_base 30..31: mod_base = OTHER; note = exNA linkage

SEQ ID NO: 8 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..4,15..17,19..20,22..25,33..36): mod_base = OTHER; note = Phosphorothioate linkage
  modified_base 24..25: mod_base = OTHER; note = exNA linkage

SEQ ID NO: 9 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..4,15..17,19..20,22..25,33..36): mod_base = OTHER; note = Phosphorothioate linkage
  modified_base 23..24: mod_base = OTHER; note = exNA linkage

SEQ ID NO: 10 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..4,15..17,19..20,22..25,33..36): mod_base = OTHER; note = Phosphorothioate linkage
  modified_base 22..23: mod_base = OTHER; note = exNA linkage

SEQ ID NO: 11 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..4,15..17,19..20,22..25,33..36): mod_base = OTHER; note = Phosphorothioate linkage
  modified_base 21..22: mod_base = OTHER; note = exNA linkage

SEQ ID NO: 12 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..4,15..17,19..20,22..25,33..36): mod_base = OTHER; note = Phosphorothioate linkage
  modified_base 17..18: mod_base = OTHER; note = exNA linkage

SEQ ID NO: 13 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..4,15..17,19..20,22..25,33..36): mod_base = OTHER; note = Phosphorothioate linkage
  modified_base 13..14: mod_base = OTHER; note = exNA linkage

SEQ ID NO: 14 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..4,15..17,19..20,22..25,33..36): mod_base = OTHER; note = Phosphorothioate linkage
  modified_base 8..9: mod_base = OTHER; note = exNA linkage

SEQ ID NO: 15 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage

SEQ ID NO: 16 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base 66..67: mod_base = OTHER; note = exNA linkage

SEQ ID NO: 17 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base 65..66: mod_base = OTHER; note = exNA linkage

SEQ ID NO: 18 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base 64..65: mod_base = OTHER; note = exNA linkage

SEQ ID NO: 19 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base 65..67: mod_base = OTHER; note = exNA linkage

SEQ ID NO: 20 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base 64..67: mod_base = OTHER; note = exNA linkage

SEQ ID NO: 21 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base 61..62: mod_base = OTHER; note = exNA linkage

SEQ ID NO: 22 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base 57..58: mod_base = OTHER; note = exNA linkage

SEQ ID NO: 23 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base 47..48: mod_base = OTHER; note = exNA linkage

SEQ ID NO: 24 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base 39..40: mod_base = OTHER; note = exNA linkage

SEQ ID NO: 25 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base 38..39: mod_base = OTHER; note = exNA linkage

SEQ ID NO: 26 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base 33..34: mod_base = OTHER; note = exNA linkage

SEQ ID NO: 27 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base 31..32: mod_base = OTHER; note = exNA linkage

SEQ ID NO: 28 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base 30..31: mod_base = OTHER; note = exNA linkage

SEQ ID NO: 29 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base 26..27: mod_base = OTHER; note = exNA linkage

SEQ ID NO: 30 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base 23..24: mod_base = OTHER; note = exNA linkage

SEQ ID NO: 31 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base 17..18: mod_base = OTHER; note = exNA linkage

SEQ ID NO: 32 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base 12..13: mod_base = OTHER; note = exNA linkage

SEQ ID NO: 33 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base 11..12: mod_base = OTHER; note = exNA linkage

SEQ ID NO: 34 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base 4..5: mod_base = OTHER; note = exNA linkage

SEQ ID NO: 35 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base order(11..12,23..24,64..67): mod_base = OTHER; note = exNA linkage

SEQ ID NO: 36 - RNA, 67 nt, synthetic construct; identical to SEQ ID NO: 2
  source 1..67: mol_type = other RNA
  modified_base order(1..11,14,16,17,19..22,25,29,32..67): mod_base = OTHER; note = 2-Prime-O-methyl RNA nucleotides
  modified_base order(1..4,64..67): mod_base = OTHER; note = Phosphorothioate linkage
  modified_base 31..32: mod_base = OTHER; note = exNA linkage

SEQ ID NO: 37 - RNA, 67 nt, synthetic construct; identical to SEQ ID NO: 2
  source 1..67: mol_type = other RNA
  modified_base order(1..11,14,16,17,19..22,25,29,31,33..67): mod_base = OTHER; note = 2-Prime-O-methyl RNA nucleotides
  modified_base order(1..4,64..67): mod_base = OTHER; note = Phosphorothioate linkage
  modified_base 30..31: mod_base = OTHER; note = exNA linkage

SEQ ID NO: 38 - RNA, 67 nt, synthetic construct; identical to SEQ ID NO: 2
  source 1..67: mol_type = other RNA
  modified_base order(1..11,14,16,17,19..22,25,27,29,33..67): mod_base = OTHER; note = 2-Prime-O-methyl RNA nucleotides
  modified_base order(1..4,64..67): mod_base = OTHER; note = Phosphorothioate linkage
  modified_base 26..27: mod_base = OTHER; note = exNA linkage

SEQ ID NO: 39 - RNA, 67 nt, synthetic construct; identical to SEQ ID NO: 2
  source 1..67: mol_type = other RNA
  modified_base order(1..11,14,16..22,25,29,33..67): mod_base = OTHER; note = 2-Prime-O-methyl RNA nucleotides
  modified_base order(1..4,64..67): mod_base = OTHER; note = Phosphorothioate linkage
  modified_base 17..18: mod_base = OTHER; note = exNA linkage

SEQ ID NO: 40 - RNA, 67 nt, synthetic construct; identical to SEQ ID NO: 2
  source 1..67: mol_type = other RNA
  modified_base order(1..11,13,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base 12..13: mod_base = OTHER; note = exNA linkage

SEQ ID NO: 41 - RNA, 67 nt, synthetic construct; identical to SEQ ID NO: 2
  source 1..67: mol_type = other RNA
  modified_base order(1..12,14,16..22,25,29,33..67): mod_base = OTHER; note = 2-Prime-O-methyl RNA nucleotides
  modified_base order(1..4,64..67): mod_base = OTHER; note = Phosphorothioate linkage
  modified_base order(11..12,17..18,64..67): mod_base = OTHER; note = exNA linkage

SEQ ID NO: 42 - RNA, 67 nt, synthetic construct; identical to SEQ ID NO: 2
  source 1..67: mol_type = other RNA
  modified_base order(1..12,14,16..22,25,29,33..67): mod_base = OTHER; note = 2-Prime-O-methyl RNA nucleotides
  modified_base order(1..4,64..67): mod_base = OTHER; note = Phosphorothioate linkage
  modified_base order(11..12,17..18,64..67): mod_base = OTHER; note = exNA linkage

SEQ ID NO: 43 - RNA, 36 nt, synthetic construct; identical to SEQ ID NO: 1
  source 1..36: mol_type = other RNA
  modified_base order(1..10,16..18,20,21,25..36): mod_base = OTHER; note = 2-Prime-O-methyl RNA nucleotides
  modified_base order(1..4,33..35): mod_base = OTHER; note = Phosphorothioate linkage

SEQ ID NO: 44 - RNA, 36 nt, synthetic construct; identical to SEQ ID NO: 1
  source 1..36: mol_type = other RNA
  modified_base order(7..10,20,21,27..36): mod_base = OTHER; note = 2-Prime-O-methyl RNA nucleotides
  modified_base 33..35: mod_base = OTHER; note = Phosphorothioate linkage

SEQ ID NO: 45 - RNA, 36 nt, synthetic construct; identical to SEQ ID NO: 1
  source 1..36: mol_type = other RNA
  modified_base order(7..10,20,21,27..36): mod_base = OTHER; note = 2-Prime-O-methyl RNA nucleotides
  modified_base order(1..4,33..35): mod_base = OTHER; note = Phosphorothioate linkage

SEQ ID NO: 46 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(1..4,33..36): mod_base = OTHER; note = Phosphorothioate linkage

SEQ ID NO: 47 - RNA, 36 nt, synthetic construct; identical to SEQ ID NO: 1
  source 1..36: mol_type = other RNA
  modified_base order(7..10,17,18,20,21,27..36): mod_base = OTHER; note = 2-Prime-O-methyl RNA nucleotides
  modified_base order(1..4,33..36): mod_base = OTHER; note = Phosphorothioate linkage

SEQ ID NO: 48 - RNA, 36 nt, synthetic construct; identical to SEQ ID NO: 1
  source 1..36: mol_type = other RNA
  modified_base order(7..10,20,21,25..36): mod_base = OTHER; note = 2-Prime-O-methyl RNA nucleotides
  modified_base order(1..4,33..36): mod_base = OTHER; note = Phosphorothioate linkage

SEQ ID NO: 49 - RNA, 36 nt, synthetic construct; identical to SEQ ID NO: 1
  source 1..36: mol_type = other RNA
  modified_base order(1..10,17,18,20,21,27..36): mod_base = OTHER; note = 2-Prime-O-methyl RNA nucleotides
  modified_base order(1..4,33..36): mod_base = OTHER; note = Phosphorothioate linkage

SEQ ID NO: 50 - RNA, 36 nt, synthetic construct; identical to SEQ ID NO: 1
  source 1..36: mol_type = other RNA
  modified_base order(1..10,20,21,25..36): mod_base = OTHER; note = 2-Prime-O-methyl RNA nucleotides
  modified_base order(1..4,33..36): mod_base = OTHER; note = Phosphorothioate linkage

SEQ ID NO: 51 - RNA, 36 nt, synthetic construct; identical to SEQ ID NO: 1
  source 1..36: mol_type = other RNA
  modified_base order(1..10,20,21,25..36): mod_base = OTHER; note = 2-Prime-O-methyl RNA nucleotides
  modified_base order(1..4,15..17,19..20,22..25,33..36): mod_base = OTHER; note = Phosphorothioate linkage

SEQ ID NO: 52 - RNA, 36 nt, synthetic construct; identical to SEQ ID NO: 1
  source 1..36: mol_type = other RNA
  modified_base order(1..10,20,21,25..36): mod_base = OTHER; note = 2-Prime-O-methyl RNA nucleotides
  modified_base order(11..14,17,18): mod_base = OTHER; note = 2-Prime-fluoro RNA nucleotides
  modified_base order(1..4,15..17,19..20,22..25,33..36): mod_base = OTHER; note = Phosphorothioate linkage

SEQ ID NO: 53 - RNA, 36 nt, synthetic construct; identical to SEQ ID NO: 1
  source 1..36: mol_type = other RNA
  modified_base order(1..10,20,21,25..36): mod_base = OTHER; note = 2-Prime-O-methyl RNA nucleotides
  modified_base 11..14: mod_base = OTHER; note = 2-Prime-fluoro RNA nucleotides
  modified_base order(1..4,15..20,22..25,33..36): mod_base = OTHER; note = Phosphorothioate linkage

SEQ ID NO: 54 - RNA, 36 nt, synthetic construct; identical to SEQ ID NO: 1
  source 1..36: mol_type = other RNA
  modified_base order(1..10,20,21,25,27..36): mod_base = OTHER; note = 2-Prime-O-methyl RNA nucleotides
  modified_base order(11..14,17,18): mod_base = OTHER; note = 2-Prime-fluoro RNA nucleotides
  modified_base order(1..4,15..17,19..20,22..25,26..27,33..36): mod_base = OTHER; note = Phosphorothioate linkage

SEQ ID NO: 55 - RNA, 36 nt, synthetic construct; identical to SEQ ID NO: 1
  source 1..36: mol_type = other RNA
  modified_base order(1..10,20,21,26..36): mod_base = OTHER; note = 2-Prime-O-methyl RNA nucleotides
  modified_base order(11..14,17,18): mod_base = OTHER; note = 2-Prime-fluoro RNA nucleotides
  modified_base order(1..4,15..17,19..20,22..26,33..36): mod_base = OTHER; note = Phosphorothioate linkage

SEQ ID NO: 56 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18): mod_base = OTHER; note = 2-Prime-fluoro RNA nucleotides
  modified_base order(1..4,15..17,19..20,22..27,33..36): mod_base = OTHER; note = Phosphorothioate linkage

SEQ ID NO: 57 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..4,15..17,19..20,22..25,33..36): mod_base = OTHER; note = Phosphorothioate linkage

SEQ ID NO: 58 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base order(1..4,33..36): mod_base = OTHER; note = Phosphorothioate linkage

SEQ ID NO: 59 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,22,24..26): mod_base = OTHER; note = 2-Prime-fluoro RNA nucleotides

SEQ ID NO: 60 - RNA, 36 nt, synthetic construct; identical to SEQ ID NO: 1
  source 1..36: mol_type = other RNA
  modified_base order(1..10,15,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..4,16..17,19..20,22..25,33..36): mod_base = OTHER; note = Phosphorothioate linkage

SEQ ID NO: 61 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,16..18,25,26): mod_base = OTHER; note = 2-Prime-fluoro RNA nucleotides
  modified_base order(1..4,15..16,19..20,22..25,33..36): mod_base = OTHER; note = Phosphorothioate linkage

SEQ ID NO: 62 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17..19,25,26): mod_base = OTHER; note = 2-Prime-fluoro RNA nucleotides
  modified_base order(1..4,15..17,22..25,33..36): mod_base = OTHER; note = Phosphorothioate linkage

SEQ ID NO: 63 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,22,25,26): mod_base = OTHER; note = 2-Prime-fluoro RNA nucleotides
  modified_base order(1..4,15..17,19..20,23..25,33..36): mod_base = OTHER; note = Phosphorothioate linkage

SEQ ID NO: 64 - RNA, 37 nt, synthetic construct
  source 1..37: mol_type = other RNA
  modified_base order(1..10,22,28..37): mod_base = OTHER; note = 2-Prime-O-methyl RNA nucleotides
  modified_base order(11..14,17,18,24,26,27): mod_base = OTHER; note = 2-Prime-fluoro RNA nucleotides
  modified_base order(1..4,15..17,19..20,23..24,25..26,34..37): mod_base = OTHER; note = Phosphorothioate linkage
nnnnnnnnnnnnnnnnnnnnagttttagagctatgct

SEQ ID NO: 65 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..4,15..17,19..20,22..24,33..36): mod_base = OTHER; note = Phosphorothioate linkage

SEQ ID NO: 66 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,16..19,22..26): mod_base = OTHER; note = 2-Prime-fluoro RNA nucleotides
  modified_base order(1..4,15..16,33..36): mod_base = OTHER; note = Phosphorothioate linkage

SEQ ID NO: 67 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(1..4,33..36): mod_base = OTHER; note = Phosphorothioate linkage

SEQ ID NO: 68 - RNA, 36 nt, synthetic construct; identical to SEQ ID NO: 1
  source 1..36: mol_type = other RNA
  modified_base order(1..3,7..11,20,21,27..36): mod_base = OTHER; note = 2-Prime-O-methyl RNA nucleotides
  modified_base order(1..4,33..36): mod_base = OTHER; note = Phosphorothioate linkage

SEQ ID NO: 69 - RNA, 36 nt, synthetic construct; identical to SEQ ID NO: 1
  source 1..36: mol_type = other RNA
  modified_base order(1..3,7..11,13,14,20,21,27..36): mod_base = OTHER; note = 2-Prime-O-methyl RNA nucleotides
  modified_base order(1..4,33..36): mod_base = OTHER; note = Phosphorothioate linkage

SEQ ID NO: 70 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(1..7,11..20,22..27,33..36): mod_base = OTHER; note = Phosphorothioate linkage

SEQ ID NO: 71 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(1..7,11..20,33..36): mod_base = OTHER; note = Phosphorothioate linkage

SEQ ID NO: 72 - RNA, 36 nt, synthetic construct; identical to SEQ ID NO: 1
  source 1..36: mol_type = other RNA
  modified_base order(1..3,7..10,20,21,25..36): mod_base = OTHER; note = 2-Prime-O-methyl RNA nucleotides
  modified_base order(1..3,33..36): mod_base = OTHER; note = Phosphorothioate linkage

SEQ ID NO: 73 - RNA, 36 nt, synthetic construct; identical to SEQ ID NO: 1
  source 1..36: mol_type = other RNA
  modified_base order(1..3,7..10,20,21,25..36): mod_base = OTHER; note = 2-Prime-O-methyl RNA nucleotides
  modified_base order(1..7,11..20,33..36): mod_base = OTHER; note = Phosphorothioate linkage

SEQ ID NO: 74 - RNA, 36 nt, synthetic construct; identical to SEQ ID NO: 1
  source 1..36: mol_type = other RNA
  modified_base order(1..3,7..10,20,21,25..36): mod_base = OTHER; note = 2-Prime-O-methyl RNA nucleotides
  modified_base order(11..14,17,18): mod_base = OTHER; note = 2-Prime-fluoro RNA nucleotides
  modified_base order(1..7,15..17,19..20,22..25,33..36): mod_base = OTHER; note = Phosphorothioate linkage

SEQ ID NO: 75 - RNA, 36 nt, synthetic construct; identical to SEQ ID NO: 1
  source 1..36: mol_type = other RNA
  modified_base order(1..3,7..10,20,21,25..36): mod_base = OTHER; note = 2-Prime-O-methyl RNA nucleotides
  modified_base 4..6: mod_base = OTHER; note = 2-Prime-deoxy RNA nucleotides
  modified_base order(11..14,17,18): mod_base = OTHER; note = 2-Prime-fluoro RNA nucleotides
  modified_base order(1..7,15..17,19..20,22..25,33..36): mod_base = OTHER; note = Phosphorothioate linkage

SEQ ID NO: 76 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base order(1..7,33..36): mod_base = OTHER; note = Phosphorothioate linkage

SEQ ID NO: 77 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..7,33..36): mod_base = OTHER; note = Phosphorothioate linkage

SEQ ID NO: 78 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(15,16,19): mod_base = OTHER; note = 2-Prime-deoxy RNA nucleotides
  modified_base order(1..4,15..17,19..20,22..25,33..36): mod_base = OTHER; note = Phosphorothioate linkage

SEQ ID NO: 79 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base 22..24: mod_base = OTHER; note = 2-Prime-deoxy RNA nucleotides
  modified_base order(1..4,15..17,19..20,22..25,33..36): mod_base = OTHER; note = Phosphorothioate linkage

SEQ ID NO: 80 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11,13,14,17,18,25,26): mod_base = OTHER; note = 2-Prime-fluoro RNA nucleotides
  modified_base order(1..4,12..13,15..17,19..20,22..25,33..36): mod_base = OTHER; note = Phosphorothioate linkage

SEQ ID NO: 81 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11,13,14,17,18,25,26): mod_base = OTHER; note = 2-Prime-fluoro RNA nucleotides
  modified_base 12: mod_base = OTHER; note = 2-Prime-deoxy RNA nucleotide
  modified_base order(1..4,12..13,15..17,19..20,22..25,33..36): mod_base = OTHER; note = Phosphorothioate linkage

SEQ ID NO: 82 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(4..6,11..19,22..26): mod_base = OTHER; note = 2-Prime-fluoro RNA nucleotides
  modified_base order(1..4,33..36): mod_base = OTHER; note = Phosphorothioate linkage

SEQ ID NO: 83 - RNA, 36 nt, synthetic construct; identical to SEQ ID NO: 1
  source 1..36: mol_type = other RNA
  modified_base order(1..10,15,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..4,16..17,19..20,22..23,24..25,33..36): mod_base = OTHER; note = Phosphorothioate linkage

SEQ ID NO: 84 - RNA, 36 nt, synthetic construct; identical to SEQ ID NO: 1
  source 1..36: mol_type = other RNA
  modified_base order(1..10,16,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..4,15..16,19..20,22..23,24..25,33..36): mod_base = OTHER; note = Phosphorothioate linkage

SEQ ID NO: 85 - RNA, 36 nt, synthetic construct; identical to SEQ ID NO: 1
  source 1..36: mol_type = other RNA
  modified_base order(1..10,16,19..21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..4,15..16,22..23,24..25,33..36): mod_base = OTHER; note = Phosphorothioate linkage

SEQ ID NO: 86 - RNA, 36 nt, synthetic construct; identical to SEQ ID NO: 1
  source 1..36: mol_type = other RNA
  modified_base order(1..10,20..22,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..4,15..17,19..20,23..25,33..36): mod_base = OTHER; note = Phosphorothioate linkage

SEQ ID NO: 87 - RNA, 36 nt, synthetic construct; identical to SEQ ID NO: 1
  source 1..36: mol_type = other RNA
  modified_base order(1..10,20,21,23,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..4,15..17,19..20,22..23,24..25,33..36): mod_base = OTHER; note = Phosphorothioate linkage

SEQ ID NO: 88 - RNA, 36 nt, synthetic construct; identical to SEQ ID NO: 1
  source 1..36: mol_type = other RNA
  modified_base order(1..10,20,21,24,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..4,15..17,19..20,22..24,33..36): mod_base = OTHER; note = Phosphorothioate linkage

SEQ ID NO: 89 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base 4..6: mod_base = OTHER; note = 2-Prime-deoxy RNA nucleotides
  modified_base order(1..7,15..17,19..20,22..27,33..36): mod_base = OTHER; note = Phosphorothioate linkage

SEQ ID NO: 90 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..8,33..36): mod_base = OTHER; note = Phosphorothioate linkage

SEQ ID NO: 91 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..7,8..9,33..36): mod_base = OTHER; note = Phosphorothioate linkage

SEQ ID NO: 92 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..7,9..10,33..36): mod_base = OTHER; note = Phosphorothioate linkage

SEQ ID NO: 93 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..7,10..11,33..36): mod_base = OTHER; note = Phosphorothioate linkage

SEQ ID NO: 94 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..7,11..12,33..36): mod_base = OTHER; note = Phosphorothioate linkage

SEQ ID NO: 95 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..7,12..13,34..36): mod_base = OTHER; note = Phosphorothioate linkage

SEQ ID NO: 96 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..7,13..14,33..36): mod_base = OTHER; note = Phosphorothioate linkage

SEQ ID NO: 97 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..7,14..15,33..36): mod_base = OTHER; note = Phosphorothioate linkage

SEQ ID NO: 98 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..7,17..18,33..36): mod_base = OTHER; note = Phosphorothioate linkage

SEQ ID NO: 99 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..7,18..19,33..36): mod_base = OTHER; note = Phosphorothioate linkage

SEQ ID NO: 100 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..7,20..21,33..36): mod_base = OTHER; note = Phosphorothioate linkage

SEQ ID NO: 101 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base 4..6: mod_base = OTHER; note = 2-Prime-deoxy RNA nucleotides
  modified_base order(1..7,21..22,33..36): mod_base = OTHER; note = Phosphorothioate linkage

SEQ ID NO: 102 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base 4..6: mod_base = OTHER; note = 2-Prime-deoxy RNA nucleotides
  modified_base order(1..7,27..28,33..36): mod_base = OTHER; note = Phosphorothioate linkage

SEQ ID NO: 103 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base 4..6: mod_base = OTHER; note = 2-Prime-deoxy RNA nucleotides
  modified_base order(1..7,28..29,33..36): mod_base = OTHER; note = Phosphorothioate linkage

SEQ ID NO: 104 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base 4..6: mod_base = OTHER; note = 2-Prime-deoxy RNA nucleotides
  modified_base order(1..7,29..30,33..36): mod_base = OTHER; note = Phosphorothioate linkage

SEQ ID NO: 105 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base 4..6: mod_base = OTHER; note = 2-Prime-deoxy RNA nucleotides
  modified_base order(1..7,30..31,33..36): mod_base = OTHER; note = Phosphorothioate linkage

SEQ ID NO: 106 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base 4..6: mod_base = OTHER; note = 2-Prime-deoxy RNA nucleotides
  modified_base order(1..7,31..32,33..36): mod_base = OTHER; note = Phosphorothioate linkage

SEQ ID NO: 107 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base 4..6: mod_base = OTHER; note = 2-Prime-deoxy RNA nucleotides
  modified_base order(1..7,32..36): mod_base = OTHER; note = Phosphorothioate linkage

SEQ ID NO: 108 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..27): mod_base = OTHER; note = 2-Prime-fluoro RNA nucleotides
  modified_base 4..6: mod_base = OTHER; note = 2-Prime-deoxy RNA nucleotides
  modified_base order(1..10,15..17,19..20,22..26,33..36): mod_base = OTHER; note = Phosphorothioate linkage

SEQ ID NO: 109 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base 4..6: mod_base = OTHER; note = 2-Prime-deoxy RNA nucleotides
  modified_base order(1..7,10..13,15..17,19..20,22..27,33..36): mod_base = OTHER; note = Phosphorothioate linkage

SEQ ID NO: 110 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base 4..6: mod_base = OTHER; note = 2-Prime-deoxy RNA nucleotides
  modified_base order(1..7,13..17,19..20,22..27,33..36): mod_base = OTHER; note = Phosphorothioate linkage

SEQ ID NO: 111 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base 4..6: mod_base = OTHER; note = 2-Prime-deoxy RNA nucleotides
  modified_base order(1..7,15..20,22..27,33..36): mod_base = OTHER; note = Phosphorothioate linkage

SEQ ID NO: 112 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base 4..6: mod_base = OTHER; note = 2-Prime-deoxy RNA nucleotides
  modified_base order(1..7,15..17,19..21,22..27,33..36): mod_base = OTHER; note = Phosphorothioate linkage

SEQ ID NO: 113 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base 4..6: mod_base = OTHER; note = 2-Prime-deoxy RNA nucleotides
  modified_base order(1..10,33..36): mod_base = OTHER; note = Phosphorothioate linkage

SEQ ID NO: 114 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base 4..6: mod_base = OTHER; note = 2-Prime-deoxy RNA nucleotides
  modified_base order(1..13,33..36): mod_base = OTHER; note = Phosphorothioate linkage

SEQ ID NO: 115 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base 4..6: mod_base = OTHER; note = 2-Prime-deoxy RNA nucleotides
  modified_base order(1..17,33..36): mod_base = OTHER; note = Phosphorothioate linkage

SEQ ID NO: 116 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base 4..6: mod_base = OTHER; note = 2-Prime-deoxy RNA nucleotides
  modified_base order(1..20,33..36): mod_base = OTHER; note = Phosphorothioate linkage

SEQ ID NO: 117 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base 4..6: mod_base = OTHER; note = 2-Prime-deoxy RNA nucleotides
  modified_base order(1..27,33..36): mod_base = OTHER; note = Phosphorothioate linkage

SEQ ID NO: 118 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base order(1..7,15..17,19..20,22..27,30..36): mod_base = OTHER; note = Phosphorothioate linkage
  modified_base 4..6: mod_base = OTHER; note = 2-Prime-deoxy RNA nucleotides

SEQ ID NO: 119 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base 4..6: mod_base = OTHER; note = 2-Prime-deoxy RNA nucleotides
  modified_base order(1..7,15..17,19..20,22..30,33..36): mod_base = OTHER; note = Phosphorothioate linkage

SEQ ID NO: 120 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base 4..6: mod_base = OTHER; note = 2-Prime-deoxy RNA nucleotides
  modified_base order(1..7,15..17,19..20,21..27,33..36): mod_base = OTHER; note = Phosphorothioate linkage

SEQ ID NO: 121 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base 4..6: mod_base = OTHER; note = 2-Prime-deoxy RNA nucleotides
  modified_base order(1..7,30..36): mod_base = OTHER; note = Phosphorothioate linkage

SEQ ID NO: 122 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base 4..6: mod_base = OTHER; note = 2-Prime-deoxy RNA nucleotides
  modified_base order(1..7,26..36): mod_base = OTHER; note = Phosphorothioate linkage

SEQ ID NO: 123 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base 4..6: mod_base = OTHER; note = 2-Prime-deoxy RNA nucleotides
  modified_base order(1..7,22..36): mod_base = OTHER; note = Phosphorothioate linkage

SEQ ID NO: 124 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base 4..6: mod_base = OTHER; note = 2-Prime-deoxy RNA nucleotides
  modified_base order(1..9,31..36): mod_base = OTHER; note = Phosphorothioate linkage

SEQ ID NO: 125 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base 4..6: mod_base = OTHER; note = 2-Prime-deoxy RNA nucleotides
  modified_base order(1..11,29..36): mod_base = OTHER; note = Phosphorothioate linkage

SEQ ID NO: 126 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base 4..6: mod_base = OTHER; note = 2-Prime-deoxy RNA nucleotides
  modified_base order(1..13,27..36): mod_base = OTHER; note = Phosphorothioate linkage

SEQ ID NO: 127 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base 4..6: mod_base = OTHER; note = 2-Prime-deoxy RNA nucleotides
  modified_base order(1..15,25..36): mod_base = OTHER; note = Phosphorothioate linkage

SEQ ID NO: 128 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base 4..6: mod_base = OTHER; note = 2-Prime-deoxy RNA nucleotides
  modified_base order(1..17,23..36): mod_base = OTHER; note = Phosphorothioate linkage

SEQ ID NO: 129 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base 4..6: mod_base = OTHER; note = 2-Prime-deoxy RNA nucleotides
  modified_base order(1..20,22..36): mod_base = OTHER; note = Phosphorothioate linkage

SEQ ID NO: 130 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base 4..6: mod_base = OTHER; note = 2-Prime-deoxy RNA nucleotides
  modified_base 1..36: mod_base = OTHER; note = Phosphorothioate linkage

SEQ ID NO: 131 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17..18,25..26): mod_base = OTHER; note = 2-Prime-fluoro RNA nucleotides
  modified_base order(1..7,15..17,19..20,22..25,33..36): mod_base = OTHER; note = Phosphorothioate linkage

SEQ ID NO: 132 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17..18,25..26): mod_base = OTHER; note = 2-Prime-fluoro RNA nucleotides
  modified_base order(1..8,15..17,19..20,22..25,33..36): mod_base = OTHER; note = Phosphorothioate linkage

SEQ ID NO: 133 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17..18,25..26): mod_base = OTHER; note = 2-Prime-fluoro RNA nucleotides
  modified_base order(1..7,15..17,19..20,22..25,30..31,33..36): mod_base = OTHER; note = Phosphorothioate linkage

SEQ ID NO: 134 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17..18,25..26): mod_base = OTHER; note = 2-Prime-fluoro RNA nucleotides
  modified_base order(1..7,15..17,19..20,22..25,31..32,33..36): mod_base = OTHER; note = Phosphorothioate linkage

SEQ ID NO: 135 - RNA, 37 nt, synthetic construct
  source 1..37: mol_type = other RNA
  modified_base order(1..10,20,22,28..37): mod_base = OTHER; note = 2-Prime-O-methyl RNA nucleotides
  modified_base order(11..14,17..18,26..27): mod_base = OTHER; note = 2-Prime-fluoro RNA nucleotides
  modified_base order(1..7,15..17,19..20,23..26,33..37): mod_base = OTHER; note = Phosphorothioate linkage
nnnnnnnnnnnnnnnnnnngngttttagagctatgct

SEQ ID NO: 136 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17..18,25..26): mod_base = OTHER; note = 2-Prime-fluoro RNA nucleotides
  modified_base order(1..7,15..17,19..20,22..27,33..36): mod_base = OTHER; note = Phosphorothioate linkage

SEQ ID NO: 137 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17..18,25..26): mod_base = OTHER; note = 2-Prime-fluoro RNA nucleotides
  modified_base order(1..7,15..17,19..20,22..27,30..36): mod_base = OTHER; note = Phosphorothioate linkage

SEQ ID NO: 138 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17..18,25..26): mod_base = OTHER; note = 2-Prime-fluoro RNA nucleotides
  modified_base order(1..7,15..17,19..20,22..30,33..36): mod_base = OTHER; note = Phosphorothioate linkage

SEQ ID NO: 139 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17..18,25..26): mod_base = OTHER; note = 2-Prime-fluoro RNA nucleotides
  modified_base order(1..7,15..17,19..20,21..27,33..36): mod_base = OTHER; note = Phosphorothioate linkage

SEQ ID NO: 140 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17..18,25..26): mod_base = OTHER; note = 2-Prime-fluoro RNA nucleotides
  modified_base order(1..7,15..17,19..20,22..25,30..36): mod_base = OTHER; note = Phosphorothioate linkage

SEQ ID NO: 141 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17..18,25..26): mod_base = OTHER; note = 2-Prime-fluoro RNA nucleotides
  modified_base order(1..7,15..17,19..20,22..25,26..36): mod_base = OTHER; note = Phosphorothioate linkage

SEQ ID NO: 142 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base order(1..8,33..36): mod_base = OTHER; note = Phosphorothioate linkage

SEQ ID NO: 143 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base order(1..7,32..36): mod_base = OTHER; note = Phosphorothioate linkage

SEQ ID NO: 144 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base order(1..7,15..20,22..26,33..36): mod_base = OTHER; note = Phosphorothioate linkage

SEQ ID NO: 145 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base order(1..7,15..17,19..20,22..27,30..36): mod_base = OTHER; note = Phosphorothioate linkage

SEQ ID NO: 146 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base order(1..7,15..17,19..20,22..30,33..36): mod_base = OTHER; note = Phosphorothioate linkage

SEQ ID NO: 147 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base order(1..7,15..17,19..20,21..27,33..36): mod_base = OTHER; note = Phosphorothioate linkage

SEQ ID NO: 148 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base order(1..7,30..32,33..36): mod_base = OTHER; note = Phosphorothioate linkage

SEQ ID NO: 149 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base order(1..7,26..32,33..36): mod_base = OTHER; note = Phosphorothioate linkage

SEQ ID NO: 150 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base order(4..6,15,16,19,22..24): mod_base = OTHER; note = 2-Prime-deoxy RNA nucleotides
  modified_base order(1..7,15..17,19..20,22..25,33..36): mod_base = OTHER; note = Phosphorothioate linkage

SEQ ID NO: 151 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17..18,25..26): mod_base = OTHER; note = 2-Prime-fluoro RNA nucleotides
  modified_base order(19,22..24): mod_base = OTHER; note = 2-Prime-NH2 (2-Prime-amino RNA) nucleotides
  modified_base order(1..4,15..17,33..36): mod_base = OTHER; note = Phosphorothioate linkage

SEQ ID NO: 152 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17..18,25..26): mod_base = OTHER; note = 2-Prime-fluoro RNA nucleotides
  modified_base 19: mod_base = OTHER; note = 2-Prime-NH2 (2-Prime-amino RNA) nucleotides
  modified_base order(1..4,15..17,22..25,33..36): mod_base = OTHER; note = Phosphorothioate linkage

SEQ ID NO: 153 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17..18,25..26): mod_base = OTHER; note = 2-Prime-fluoro RNA nucleotides
  modified_base order(1..4,15..17,19..20,23..25,33..36): mod_base = OTHER; note = Phosphorothioate linkage
  modified_base 22: mod_base = OTHER; note = 2-Prime-NH2 (2-Prime-amino RNA) nucleotides

SEQ ID NO: 154 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17..18,25..26): mod_base = OTHER; note = 2-Prime-fluoro RNA nucleotides
  modified_base order(1..4,15..17,19..20,22..23,24..25,33..36): mod_base = OTHER; note = Phosphorothioate linkage
  modified_base 23: mod_base = OTHER; note = 2-Prime-NH2 (2-Prime-amino RNA) nucleotides

SEQ ID NO: 155 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17..18,25..26): mod_base = OTHER; note = 2-Prime-fluoro RNA nucleotides
  modified_base order(1..4,15..17,19..20,22..24,33..36): mod_base = OTHER; note = Phosphorothioate linkage
  modified_base 24: mod_base = OTHER; note = 2-Prime-NH2 (2-Prime-amino RNA) nucleotides

SEQ ID NO: 156 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17..18,25..26): mod_base = OTHER; note = 2-Prime-fluoro RNA nucleotides
  modified_base order(19,22..24): mod_base = OTHER; note = 4-Prime-thio RNA nucleotides
  modified_base order(1..4,15..17,19..20,22..25,33..36): mod_base = OTHER; note = Phosphorothioate linkage

SEQ ID NO: 157 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17..18,25..26): mod_base = OTHER; note = 2-Prime-fluoro RNA nucleotides
  modified_base order(19,22..24): mod_base = OTHER; note = 4-Prime-thio RNA nucleotides
  modified_base order(1..4,15..17,33..36): mod_base = OTHER; note = Phosphorothioate linkage

SEQ ID NO: 158 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17..18,25..26): mod_base = OTHER; note = 2-Prime-fluoro RNA nucleotides
  modified_base order(1..4,15..17,22..25,33..36): mod_base = OTHER; note = Phosphorothioate linkage
  modified_base 19: mod_base = OTHER; note = 4-Prime-thio RNA nucleotides

SEQ ID NO: 159 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17..18,25..26): mod_base = OTHER; note = 2-Prime-fluoro RNA nucleotides
  modified_base order(1..4,15..17,19..20,23..25,33..36): mod_base = OTHER; note = Phosphorothioate linkage
  modified_base 22: mod_base = OTHER; note = 4-Prime-thio RNA nucleotides

SEQ ID NO: 160 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17..18,25..26): mod_base = OTHER; note = 2-Prime-fluoro RNA nucleotides
  modified_base order(1..4,15..17,19..20,22..23,24..25,33..36): mod_base = OTHER; note = Phosphorothioate linkage
  modified_base 23: mod_base = OTHER; note = 4-Prime-thio RNA nucleotides

SEQ ID NO: 161 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17..18,25..26): mod_base = OTHER; note = 2-Prime-fluoro RNA nucleotides
  modified_base 24: mod_base = OTHER; note = 4-Prime-thio RNA nucleotides
  modified_base order(1..4,15..17,19..20,22..24,33..36): mod_base = OTHER; note = Phosphorothioate linkage

SEQ ID NO: 162 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17..18,25..26): mod_base = OTHER; note = 2-Prime-fluoro RNA nucleotides
  modified_base order(22,24): mod_base = OTHER; note = 4-Prime-thio RNA nucleotides
  modified_base order(1..4,15..17,19..20,23..24,33..36): mod_base = OTHER; note = Phosphorothioate linkage

SEQ ID NO: 163 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17..18,25..26): mod_base = OTHER; note = 2-Prime-fluoro RNA nucleotides
  modified_base 22..23: mod_base = OTHER; note = 4-Prime-thio RNA nucleotides
  modified_base order(1..4,15..17,19..20,24..25,33..36): mod_base = OTHER; note = Phosphorothioate linkage

SEQ ID NO: 164 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17..18,25..26): mod_base = OTHER; note = 2-Prime-fluoro RNA nucleotides
  modified_base 23..24: mod_base = OTHER; note = 4-Prime-thio RNA nucleotides
  modified_base order(1..4,15..17,19..20,22..23,33..36): mod_base = OTHER; note = Phosphorothioate linkage

SEQ ID NO: 165 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17..18,25..26): mod_base = OTHER; note = 2-Prime-fluoro RNA nucleotides
  modified_base 22..24: mod_base = OTHER; note = 2-Prime-NH2 (2-Prime-amino RNA) nucleotides
  modified_base order(1..4,15..17,19..20,33..36): mod_base = OTHER; note = Phosphorothioate linkage

SEQ ID NO: 166 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17..18,25..26): mod_base = OTHER; note = 2-Prime-fluoro RNA nucleotides
  modified_base 22..24: mod_base = OTHER; note = 4-Prime-thio RNA nucleotides
  modified_base order(1..4,15..17,19..20,22..25,33..36): mod_base = OTHER; note = Phosphorothioate linkage

SEQ ID NO: 167 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17..18,25..26): mod_base = OTHER; note = 2-Prime-fluoro RNA nucleotides
  modified_base 22..24: mod_base = OTHER; note = 4-Prime-thio RNA nucleotides
  modified_base order(1..4,15..17,19..20,33..36): mod_base = OTHER; note = Phosphorothioate linkage

SEQ ID NO: 168 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17..18,25..26): mod_base = OTHER; note = 2-Prime-fluoro RNA nucleotides
  modified_base order(1..4,15..16,19..20,22..25,33..36): mod_base = OTHER; note = Phosphorothioate linkage
  modified_base 16: mod_base = OTHER; note = 2-Prime-NH2 (2-Prime-amino RNA) nucleotides

SEQ ID NO: 169 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17..18,25..26): mod_base = OTHER; note = 2-Prime-fluoro RNA nucleotides
  modified_base 19: mod_base = OTHER; note = 2-Prime-NH2 (2-Prime-amino RNA) nucleotides
  modified_base order(1..4,15..17,22..25,33..36): mod_base = OTHER; note = Phosphorothioate linkage

SEQ ID NO: 170 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17..18,25..26): mod_base = OTHER; note = 2-Prime-fluoro RNA nucleotides
  modified_base order(16,19): mod_base = OTHER; note = 2-Prime-NH2 (2-Prime-amino RNA) nucleotides
  modified_base order(1..4,15..17,19..20,22..25,33..36): mod_base = OTHER; note = Phosphorothioate linkage

SEQ ID NO: 171 - RNA, 67 nt, synthetic construct; identical to SEQ ID NO: 2
  source 1..67: mol_type = other RNA
  modified_base order(1..7,14,22,25,29,37..48,53..55,61..67): mod_base = OTHER; note = 2-Prime-O-methyl RNA nucleotides
  modified_base order(1..4,64..67): mod_base = OTHER; note = Phosphorothioate linkage

SEQ ID NO: 172 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,12..14,15..16,18..19,23..25,26..29,30..33,64..67): mod_base = OTHER; note = Phosphorothioate linkage

SEQ ID NO: 173 - RNA, 67 nt, synthetic construct; identical to SEQ ID NO: 2
  source 1..67: mol_type = other RNA
  modified_base order(1..11,14..17,19..22,25,29,33..67): mod_base = OTHER; note = 2-Prime-O-methyl RNA nucleotides
  modified_base order(1..4,64..67): mod_base = OTHER; note = Phosphorothioate linkage

SEQ ID NO: 174 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base order(18,23..24,27..28): mod_base = OTHER; note = 2-Prime-fluoro RNA nucleotides

SEQ ID NO: 175 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(13,18,23..24,26..28): mod_base = OTHER; note = 2-Prime-fluoro RNA nucleotides
  modified_base order(1..4,64..67): mod_base = OTHER; note = Phosphorothioate linkage

SEQ ID NO: 176 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(12..13,15,18,23..24,26..28,30..32): mod_base = OTHER; note = 2-Prime-fluoro RNA nucleotides
  modified_base order(1..4,64..67): mod_base = OTHER; note = Phosphorothioate linkage

SEQ ID NO: 177 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base 12: mod_base = OTHER; note = 2-Prime-fluoro RNA nucleotides
  modified_base order(1..4,64..67): mod_base = OTHER; note = Phosphorothioate linkage

SEQ ID NO: 178 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base 13: mod_base = OTHER; note = 2-Prime-fluoro RNA nucleotides
  modified_base order(1..4,64..67): mod_base = OTHER; note = Phosphorothioate linkage

SEQ ID NO: 179 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base 15: mod_base = OTHER; note = 2-Prime-fluoro RNA nucleotides
  modified_base order(1..4,64..67): mod_base = OTHER; note = Phosphorothioate linkage

SEQ ID NO: 180 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base 18: mod_base = OTHER; note = 2-Prime-fluoro RNA nucleotides
  modified_base order(1..4,64..67): mod_base = OTHER; note = Phosphorothioate linkage

SEQ ID NO: 181 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base 23: mod_base = OTHER; note = 2-Prime-fluoro RNA nucleotides
  modified_base order(1..4,64..67): mod_base = OTHER; note = Phosphorothioate linkage

SEQ ID NO: 182 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base 24: mod_base = OTHER; note = 2-Prime-fluoro RNA nucleotides
  modified_base order(1..4,64..67): mod_base = OTHER; note = Phosphorothioate linkage

SEQ ID NO: 183 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base 26: mod_base = OTHER; note = 2-Prime-fluoro RNA nucleotides
  modified_base order(1..4,64..67): mod_base = OTHER; note = Phosphorothioate linkage

SEQ ID NO: 184 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base 27: mod_base = OTHER; note = 2-Prime-fluoro RNA nucleotides
  modified_base order(1..4,64..67): mod_base = OTHER; note = Phosphorothioate linkage

SEQ ID NO: 185 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base 28: mod_base = OTHER; note = 2-Prime-fluoro RNA nucleotides
  modified_base order(1..4,64..67): mod_base = OTHER; note = Phosphorothioate linkage

SEQ ID NO: 186 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base 30: mod_base = OTHER; note = 2-Prime-fluoro RNA nucleotides
  modified_base order(1..4,64..67): mod_base = OTHER; note = Phosphorothioate linkage

SEQ ID NO: 187 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base 31: mod_base = OTHER; note = 2-Prime-O-methyl RNA nucleotides
  modified_base order(1..4,64..67): mod_base = OTHER; note = Phosphorothioate linkage

SEQ ID NO: 188 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base 32: mod_base = OTHER; note = 2-Prime-fluoro RNA nucleotides
  modified_base order(1..4,64..67): mod_base = OTHER; note = Phosphorothioate linkage

SEQ ID NO: 189 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(18,23..24,27,28,30..32): mod_base = OTHER; note = 2-Prime-fluoro RNA nucleotides
  modified_base order(1..4,64..67): mod_base = OTHER; note = Phosphorothioate linkage

SEQ ID NO: 190 - RNA, 67 nt, synthetic construct; identical to SEQ ID NO: 2
  source 1..67: mol_type = other RNA
  modified_base order(1..12,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage

SEQ ID NO: 191 - RNA, 67 nt, synthetic construct; identical to SEQ ID NO: 2
  source 1..67: mol_type = other RNA
  modified_base order(1..11,13,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage

SEQ ID NO: 192 - RNA, 67 nt, synthetic construct; identical to SEQ ID NO: 2
  source 1..67: mol_type = other RNA
  modified_base order(1..11,14,16..22,25,29,33..67): mod_base = OTHER; note = 2-Prime-O-methyl RNA nucleotides
  modified_base order(1..4,64..67): mod_base = OTHER; note = Phosphorothioate linkage

SEQ ID NO: 193 - RNA, 67 nt, synthetic construct; identical to SEQ ID NO: 2
  source 1..67: mol_type = other RNA
  modified_base order(1..11,14,16,17,19..23,25,29,33..67): mod_base = OTHER; note = 2-Prime-O-methyl RNA nucleotides
  modified_base order(1..4,64..67): mod_base = OTHER; note = Phosphorothioate linkage

SEQ ID NO: 194 - RNA, 67 nt, synthetic construct; identical to SEQ ID NO: 2
  source 1..67: mol_type = other RNA
  modified_base order(1..11,14,16,17,19..22,24,25,29,33..67): mod_base = OTHER; note = 2-Prime-O-methyl RNA nucleotides
  modified_base order(1..4,64..67): mod_base = OTHER; note = Phosphorothioate linkage

SEQ ID NO: 195 - RNA, 67 nt, synthetic construct; identical to SEQ ID NO: 2
  source 1..67: mol_type = other RNA
  modified_base order(1..11,14,16,17,19..22,25,26,29,33..67): mod_base = OTHER; note = 2-Prime-O-methyl RNA nucleotides
  modified_base order(1..4,64..67): mod_base = OTHER; note = Phosphorothioate linkage

SEQ ID NO: 196 - RNA, 67 nt, synthetic construct; identical to SEQ ID NO: 2
  source 1..67: mol_type = other RNA
  modified_base order(1..11,14,16,17,19..22,25,27,29,33..67): mod_base = OTHER; note = 2-Prime-O-methyl RNA nucleotides
  modified_base order(1..4,64..67): mod_base = OTHER; note = Phosphorothioate linkage

SEQ ID NO: 197 - RNA, 67 nt, synthetic construct; identical to SEQ ID NO: 2
  source 1..67: mol_type = other RNA
  modified_base order(1..11,14,16,17,19..22,25,28,29,33..67): mod_base = OTHER; note = 2-Prime-O-methyl RNA nucleotides
  modified_base order(1..4,64..67): mod_base = OTHER; note = Phosphorothioate linkage

SEQ ID NO: 198 - RNA, 67 nt, synthetic construct; identical to SEQ ID NO: 2
  source 1..67: mol_type = other RNA
  modified_base order(1..11,14,16,17,19..22,25,29,30,33..67): mod_base = OTHER; note = 2-Prime-O-methyl RNA nucleotides
  modified_base order(1..4,64..67): mod_base = OTHER; note = Phosphorothioate linkage

SEQ ID NO: 199 - RNA, 67 nt, synthetic construct; identical to SEQ ID NO: 2
  source 1..67: mol_type = other RNA
  modified_base order(1..11,14,16,17,19..22,25,29,31,33..67): mod_base = OTHER; note = 2-Prime-O-methyl RNA nucleotides
  modified_base order(1..4,64..67): mod_base = OTHER; note = Phosphorothioate linkage

SEQ ID NO: 200 - RNA, 67 nt, synthetic construct; identical to SEQ ID NO: 2
  source 1..67: mol_type = other RNA
  modified_base order(1..11,14,16,17,19..22,25,29,32..67): mod_base = OTHER; note = 2-Prime-O-methyl RNA nucleotides
  modified_base order(1..4,64..67): mod_base = OTHER; note = Phosphorothioate linkage

SEQ ID NO: 201 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,12..13,64..67): mod_base = OTHER; note = Phosphorothioate linkage

SEQ ID NO: 202 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,13..14,64..67): mod_base = OTHER; note = Phosphorothioate linkage

SEQ ID NO: 203 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,15..16,64..67): mod_base = OTHER; note = Phosphorothioate linkage

SEQ ID NO: 204 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,18..19,64..67): mod_base = OTHER; note = Phosphorothioate linkage

SEQ ID NO: 205 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,23..24,64..67): mod_base = OTHER; note = Phosphorothioate linkage

SEQ ID NO: 206 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,24..25,64..67): mod_base = OTHER; note = Phosphorothioate linkage

SEQ ID NO: 207 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,26..27,64..67): mod_base = OTHER; note = Phosphorothioate linkage

SEQ ID NO: 208 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,27..28,64..67): mod_base = OTHER; note = Phosphorothioate linkage

SEQ ID NO: 209 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,28..29,64..67): mod_base = OTHER; note = Phosphorothioate linkage

SEQ ID NO: 210 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,30..31,64..67): mod_base = OTHER; note = Phosphorothioate linkage

SEQ ID NO: 211 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,31..32,64..67): mod_base = OTHER; note = Phosphorothioate linkage

SEQ ID NO: 212 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,32..33,64..67): mod_base = OTHER; note = Phosphorothioate linkage

SEQ ID NO: 213 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base 12..13: mod_base = OTHER; note = 2-Prime-fluoro RNA nucleotides

SEQ ID NO: 214 - RNA, 67 nt, synthetic construct; identical to SEQ ID NO: 2
  source 1..67: mol_type = other RNA
  modified_base order(1..11,14,16..22,25,29,33..67): mod_base = OTHER; note = 2-Prime-O-methyl RNA nucleotides
  modified_base order(1..4,64..67): mod_base = OTHER; note = Phosphorothioate linkage
  modified_base 15: mod_base = OTHER; note = 2-Prime-fluoro RNA nucleotides

SEQ ID NO: 215 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base order(12,13,15,18): mod_base = OTHER; note = 2-Prime-fluoro RNA nucleotides

SEQ ID NO: 216 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base 23..24: mod_base = OTHER; note = 2-Prime-fluoro RNA nucleotides
  modified_base order(1..4,64..67): mod_base = OTHER; note = Phosphorothioate linkage

SEQ ID NO: 217 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base 26..28: mod_base = OTHER; note = 2-Prime-fluoro RNA nucleotides

SEQ ID NO: 218 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base 30..32: mod_base = OTHER; note = 2-Prime-fluoro RNA nucleotides

SEQ ID NO: 219 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base order(23,24,30..32): mod_base = OTHER; note = 2-Prime-fluoro RNA nucleotides

SEQ ID NO: 220 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base order(23,24,26..28): mod_base = OTHER; note = 2-Prime-fluoro RNA nucleotides

SEQ ID NO: 221 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base order(26..28,30..32): mod_base = OTHER; note = 2-Prime-fluoro RNA nucleotides

SEQ ID NO: 222 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base order(30,32): mod_base = OTHER; note = 2-Prime-fluoro RNA nucleotides

SEQ ID NO: 223 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base order(23,24,26,28,30,32): mod_base = OTHER; note = 2-Prime-fluoro RNA nucleotides

SEQ ID NO: 224 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base order(26,28,30,32): mod_base = OTHER; note = 2-Prime-fluoro RNA nucleotides

SEQ ID NO: 225 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base order(23,26,28,30,32): mod_base = OTHER; note = 2-Prime-fluoro RNA nucleotides

SEQ ID NO: 226 - RNA, 67 nt, synthetic construct; identical to SEQ ID NO: 2
  source 1..67: mol_type = other RNA
  modified_base order(1..14,16,17,19..22,25,29,33..67): mod_base = OTHER; note = 2-Prime-O-methyl RNA nucleotides
  modified_base order(1..4,64..67): mod_base = OTHER; note = Phosphorothioate linkage

SEQ ID NO: 227 - RNA, 67 nt, synthetic construct; identical to SEQ ID NO: 2
  source 1..67: mol_type = other RNA
  modified_base order(1..11,14..22,25,29,33..67): mod_base = OTHER; note = 2-Prime-O-methyl RNA nucleotides
  modified_base order(1..4,64..67): mod_base = OTHER; note = Phosphorothioate linkage

SEQ ID NO: 228 - RNA, 67 nt, synthetic construct; identical to SEQ ID NO: 2
  source 1..67: mol_type = other RNA
  modified_base order(1..22,25,29,33..67): mod_base = OTHER; note = 2-Prime-O-methyl RNA nucleotides
  modified_base order(1..4,64..67): mod_base = OTHER; note = Phosphorothioate linkage

SEQ ID NO: 229 - RNA, 67 nt, synthetic construct; identical to SEQ ID NO: 2
  source 1..67: mol_type = other RNA
  modified_base order(1..11,14,16,17,19..25,29,33..67): mod_base = OTHER; note = 2-Prime-O-methyl RNA nucleotides
  modified_base order(1..4,64..67): mod_base = OTHER; note = Phosphorothioate linkage

SEQ ID NO: 230 - RNA, 67 nt, synthetic construct; identical to SEQ ID NO: 2
  source 1..67: mol_type = other RNA
  modified_base order(1..11,14,16,17,19..22,25..29,33..67): mod_base = OTHER; note = 2-Prime-O-methyl RNA nucleotides
  modified_base order(1..4,64..67): mod_base = OTHER; note = Phosphorothioate linkage

SEQ ID NO: 231 - RNA, 67 nt, synthetic construct; identical to SEQ ID NO: 2
  source 1..67: mol_type = other RNA
  modified_base order(1..11,14,16,17,19..22,25,29..67): mod_base = OTHER; note = 2-Prime-O-methyl RNA nucleotides
  modified_base order(1..4,64..67): mod_base = OTHER; note = Phosphorothioate linkage

SEQ ID NO: 232 - RNA, 67 nt, synthetic construct; identical to SEQ ID NO: 2
  source 1..67: mol_type = other RNA
  modified_base order(1..11,14,16,17,19..25,29..67): mod_base = OTHER; note = 2-Prime-O-methyl RNA nucleotides
  modified_base order(1..4,64..67): mod_base = OTHER; note = Phosphorothioate linkage

SEQ ID NO: 233 - RNA, 67 nt, synthetic construct; identical to SEQ ID NO: 2
  source 1..67: mol_type = other RNA
  modified_base order(1..11,14,16,17,19..29,33..67): mod_base = OTHER; note = 2-Prime-O-methyl RNA nucleotides
  modified_base order(1..4,64..67): mod_base = OTHER; note = Phosphorothioate linkage

SEQ ID NO: 234 - RNA, 67 nt, synthetic construct; identical to SEQ ID NO: 2
  source 1..67: mol_type = other RNA
  modified_base order(1..11,14,16,17,19..22,25..67): mod_base = OTHER; note = 2-Prime-O-methyl RNA nucleotides
  modified_base order(1..4,64..67): mod_base = OTHER; note = Phosphorothioate linkage

SEQ ID NO: 235 - RNA, 67 nt, synthetic construct; identical to SEQ ID NO: 2
  source 1..67: mol_type = other RNA
  modified_base order(1..11,14,16,17,19..22,25,26,28,29,33..67): mod_base = OTHER; note = 2-Prime-O-methyl RNA nucleotides
  modified_base order(1..4,64..67): mod_base = OTHER; note = Phosphorothioate linkage

SEQ ID NO: 236 - RNA, 67 nt, synthetic construct; identical to SEQ ID NO: 2
  source 1..67: mol_type = other RNA
  modified_base order(1..11,14,16,17,19..22,25,29,30,32..67): mod_base = OTHER; note = 2-Prime-O-methyl RNA nucleotides
  modified_base order(1..4,64..67): mod_base = OTHER; note = Phosphorothioate linkage

SEQ ID NO: 237 - RNA, 67 nt, synthetic construct; identical to SEQ ID NO: 2
  source 1..67: mol_type = other RNA
  modified_base order(1..11,14,16,17,19..26,28..30,32..67): mod_base = OTHER; note = 2-Prime-O-methyl RNA nucleotides
  modified_base order(1..4,64..67): mod_base = OTHER; note = Phosphorothioate linkage

SEQ ID NO: 238 - RNA, 67 nt, synthetic construct; identical to SEQ ID NO: 2
  source 1..67: mol_type = other RNA
  modified_base order(1..11,14,16,17,19..22,25,26,28..30,32..67): mod_base = OTHER; note = 2-Prime-O-methyl RNA nucleotides
  modified_base order(1..4,64..67): mod_base = OTHER; note = Phosphorothioate linkage

SEQ ID NO: 239 - RNA, 67 nt, synthetic construct; identical to SEQ ID NO: 2
  source 1..67: mol_type = other RNA
  modified_base order(1..11,14,16,17,19..23,25,26,28..30,32..67): mod_base = OTHER; note = 2-Prime-O-methyl RNA nucleotides
  modified_base order(1..4,64..67): mod_base = OTHER; note = Phosphorothioate linkage

SEQ ID NO: 240 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base 12..13: mod_base = OTHER; note = 2-Prime-deoxy RNA nucleotides

SEQ ID NO: 241 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base order(15,18): mod_base = OTHER; note = 2-Prime-deoxy RNA nucleotides

SEQ ID NO: 242 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base order(12,13,15,18): mod_base = OTHER; note = 2-Prime-deoxy RNA nucleotides

SEQ ID NO: 243 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base 23..24: mod_base = OTHER; note = 2-Prime-deoxy RNA nucleotides

SEQ ID NO: 244 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base 26..28: mod_base = OTHER; note = 2-Prime-deoxy RNA nucleotides

SEQ ID NO: 245 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base 30..32: mod_base = OTHER; note = 2-Prime-deoxy RNA nucleotides

SEQ ID NO: 246 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base order(23,24,30..32): mod_base = OTHER; note = 2-Prime-deoxy RNA nucleotides

SEQ ID NO: 247 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base order(23,24,26..28): mod_base = OTHER; note = 2-Prime-deoxy RNA nucleotides

SEQ ID NO: 248 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base order(26..28,30..32): mod_base = OTHER; note = 2-Prime-deoxy RNA nucleotides

SEQ ID NO: 249 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base order(26,28): mod_base = OTHER; note = 2-Prime-deoxy RNA nucleotides

SEQ ID NO: 250 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base order(30,32): mod_base = OTHER; note = 2-Prime-deoxy RNA nucleotides

SEQ ID NO: 251 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base order(23,24,26,28,30,32): mod_base = OTHER; note = 2-Prime-deoxy RNA nucleotides

SEQ ID NO: 252 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base order(26,28,30,32): mod_base = OTHER; note = 2-Prime-deoxy RNA nucleotides

SEQ ID NO: 253 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base order(1..4,64..67): mod_base = OTHER; note = Phosphorothioate linkage
  modified_base order(23,26,28,30,32): mod_base = OTHER; note = 2-Prime-deoxy RNA nucleotides

SEQ ID NO: 254 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,12..14,64..67): mod_base = OTHER; note = Phosphorothioate linkage

SEQ ID NO: 255 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,15..16,18..19,64..67): mod_base = OTHER; note = Phosphorothioate linkage

SEQ ID NO: 256 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,12..14,15..16,18..19,64..67): mod_base = OTHER; note = Phosphorothioate linkage

SEQ ID NO: 257 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,23..25,64..67): mod_base = OTHER; note = Phosphorothioate linkage

SEQ ID NO: 258 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,26..29,64..67): mod_base = OTHER; note = Phosphorothioate linkage

SEQ ID NO: 259 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,30..33,64..67): mod_base = OTHER; note = Phosphorothioate linkage

SEQ ID NO: 260 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,23..25,30..33,64..67): mod_base = OTHER; note = Phosphorothioate linkage

SEQ ID NO: 261 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,23..25,26..29,64..67): mod_base = OTHER; note = Phosphorothioate linkage

SEQ ID NO: 262 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,26..29,30..33,64..67): mod_base = OTHER; note = Phosphorothioate linkage

SEQ ID NO: 263 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,26..27,28..29,64..67): mod_base = OTHER; note = Phosphorothioate linkage

SEQ ID NO: 264 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,30..31,32..33,64..67): mod_base = OTHER; note = Phosphorothioate linkage

SEQ ID NO: 265 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,23..25,26..28,30..31,32..33,64..67): mod_base = OTHER; note = Phosphorothioate linkage

SEQ ID NO: 266 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,26..28,30..31,32..33,64..67): mod_base = OTHER; note = Phosphorothioate linkage

SEQ ID NO: 267 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,23..24,26..28,30..31,32..33,64..67): mod_base = OTHER; note = Phosphorothioate linkage

SEQ ID NO: 268 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base order(18,23,24,27,28): mod_base = OTHER; note = 2-Prime-deoxy RNA nucleotides

SEQ ID NO: 269 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base order(13,18,23,24,26,28): mod_base = OTHER; note = 2-Prime-deoxy RNA nucleotides

SEQ ID NO: 270 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base order(12,13,15,18,23,24,26..28,30..32): mod_base = OTHER; note = 2-Prime-deoxy RNA nucleotides

SEQ ID NO: 271 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base order(12,13,18,24,27,31,32): mod_base = OTHER; note = 4-Prime-thio RNA nucleotides

SEQ ID NO: 272 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base order(12,13,18): mod_base = OTHER; note = 4-Prime-thio RNA nucleotides

SEQ ID NO: 273 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base order(24,27,31,32): mod_base = OTHER; note = 4-Prime-thio RNA nucleotides

SEQ ID NO: 274 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base 12: mod_base = OTHER; note = 4-Prime-thio RNA nucleotides

SEQ ID NO: 275 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base 13: mod_base = OTHER; note = 4-Prime-thio RNA nucleotides

SEQ ID NO: 276 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base 18: mod_base = OTHER; note = 4-Prime-thio RNA nucleotides

SEQ ID NO: 277 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base 24: mod_base = OTHER; note = 4-Prime-thio RNA nucleotides

SEQ ID NO: 278 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base 27: mod_base = OTHER; note = 4-Prime-thio RNA nucleotides

SEQ ID NO: 279 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base 31: mod_base = OTHER; note = 4-Prime-thio RNA nucleotides

SEQ ID NO: 280 - RNA, 67 nt, synthetic construct; identical to SEQ ID NO: 2
  source 1..67: mol_type = other RNA
  modified_base order(1..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base 32: mod_base = OTHER; note = 4-Prime-thio RNA nucleotides

SEQ ID NO: 281 - RNA, 36 nt, synthetic construct; identical to SEQ ID NO: 3
  source 1..36: mol_type = other RNA
  modified_base order(21,27,28,35,36): mod_base = OTHER; note = 2-Prime-O-methyl RNA nucleotides
  modified_base order(25,26,29..34): mod_base = OTHER; note = 2-Prime-fluoro RNA nucleotides
  modified_base order(22..25,33..36): mod_base = OTHER; note = Phosphorothioate linkage

SEQ ID NO: 282 - RNA, 67 nt, synthetic construct; identical to SEQ ID NO: 4
  source 1..67: mol_type = other RNA
  modified_base order(1,2,9..11,14,16,17,19..22,25,29,33..67): mod_base = OTHER; note = 2-Prime-O-methyl RNA nucleotides
  modified_base order(1..4,64..67): mod_base = OTHER; note = Phosphorothioate linkage
  modified_base 3..8: mod_base = OTHER; note = 2-Prime-fluoro RNA nucleotides

SEQ ID NO: 283 - RNA, 36 nt, synthetic construct
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..4,15..17,19..20,22..25,33..36): mod_base = OTHER; note = Phosphorothioate linkage
gagacaaatcacctgcctcggttttagagctatgct

SEQ ID NO: 284 - RNA, 36 nt, synthetic construct; identical to SEQ ID NO: 283
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..4,15..17,19..20,22..25,33..36): mod_base = OTHER; note = Phosphorothioate linkage
  modified_base 35..36: mod_base = OTHER; note = exNA linkage

SEQ ID NO: 285 - RNA, 36 nt, synthetic construct; identical to SEQ ID NO: 283
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base 32..33: mod_base = OTHER; note = exNA linkage
  modified_base order(1..4,15..17,19..20,22..25,33..36): mod_base = OTHER; note = Phosphorothioate linkage

SEQ ID NO: 286 - RNA, 36 nt, synthetic construct; identical to SEQ ID NO: 283
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..4,15..17,19..20,22..25,33..36): mod_base = OTHER; note = Phosphorothioate linkage
  modified_base 30..31: mod_base = OTHER; note = exNA linkage

SEQ ID NO: 287 - RNA, 36 nt, synthetic construct; identical to SEQ ID NO: 283
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..4,15..17,19,22..25,33..36): mod_base = OTHER; note = Phosphorothioate linkage
  modified_base 24..25: mod_base = OTHER; note = exNA linkage

SEQ ID NO: 288 - RNA, 36 nt, synthetic construct; identical to SEQ ID NO: 283
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..4,15..17,19..20,22..25,33..36): mod_base = OTHER; note = Phosphorothioate linkage
  modified_base 23..24: mod_base = OTHER; note = exNA linkage

SEQ ID NO: 289 - RNA, 36 nt, synthetic construct; identical to SEQ ID NO: 283
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..4,15..17,19..20,22..25,33..36): mod_base = OTHER; note = Phosphorothioate linkage
  modified_base 22..23: mod_base = OTHER; note = exNA linkage

SEQ ID NO: 290 - RNA, 36 nt, synthetic construct; identical to SEQ ID NO: 283
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..4,15..17,19..20,22..25,33..36): mod_base = OTHER; note = Phosphorothioate linkage
  modified_base 21..22: mod_base = OTHER; note = exNA linkage

SEQ ID NO: 291 - RNA, 36 nt, synthetic construct; identical to SEQ ID NO: 283
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..4,15..17,19..20,22..25,33..36): mod_base = OTHER; note = Phosphorothioate linkage
  modified_base 17..18: mod_base = OTHER; note = exNA linkage

SEQ ID NO: 292 - RNA, 36 nt, synthetic construct; identical to SEQ ID NO: 283
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..4,15..17,19..20,22..25,33..36): mod_base = OTHER; note = Phosphorothioate linkage
  modified_base 13..14: mod_base = OTHER; note = exNA linkage

SEQ ID NO: 293 - RNA, 36 nt, synthetic construct; identical to SEQ ID NO: 283
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..4,15..17,19..20,22..25,33..36): mod_base = OTHER; note = Phosphorothioate linkage
  modified_base 8..9: mod_base = OTHER; note = exNA linkage

SEQ ID NO: 294 - RNA, 20 nt, synthetic construct
  source 1..20: mol_type = other RNA
gagacaaatcacctgcctcg

SEQ ID NO: 295 - RNA, 20 nt, synthetic construct
  source 1..20: mol_type = other RNA
tttaccgtattccacgaggc